(19) United States
(12) Patent Application Publication
(10) Pub. No.: US 2026/0294970 A1
(43) Pub. Date: Oct. 1, 2026

(54) CHIMERIC CYTOKINE RECEPTORS AND METHODS OF USE THEREOF

(71) Applicant: Legend Biotech USA Inc., Somerset, NJ (US)

(72) Inventors: Qingling Jiang, Nanjing (CN); Yafeng ZHANG, Nanjing (CN); Chunjing Wu, Nanjing (CN)

(21) Appl. No.: 19/480,131

(22) PCT Filed: Jul. 31, 2024
(86) PCT No.: PCT/CN2024/108770

§ 371 Date: Oct. 30, 2025

(30) Foreign Application Priority Data
Jul. 31, 2023 (WO) PCT/CN2023/110260

(51) Int. Cl. A61K 35/17; A61K 9/00; A61K 31/7088; A61K 38/00; A61K 40/11; A61K 40/31; A61K 40/32; A61K 40/42; A61P 35/00; C07K 14/705; C07K 14/715; C07K 16/18; C07K 16/40; C12N 5/0783; C12N 15/63

(52) U.S. Cl. CPC A61K 35/17; A61K 9/0019; A61K 31/7088; A61K 38/00; A61K 40/11; A61K 40/31; A61K 40/32; A61K 40/42; A61P 35/00; C07K 14/7051; C07K 14/70517; C07K 14/70521; C07K 14/715; C07K 16/18; C07K 16/40; C12N 5/0636; C12N 15/63; A61K 2239/31; A61K 2239/48; C07K 2317/565; C07K 2317/622; C07K 2319/03

(57) ABSTRACT

Provided are chimeric cytokine receptors, immune cells expressing the chimeric cytokine receptors and uses thereof.

### CROSS REFERENCE TO RELATED APPLICATION

This application claims priority benefits of International Application No. PCT/CN2023/110260, filed on Jul. 31, 2023, the contents of which are incorporated herein by reference in its entirety.

### SEQUENCE STATEMENT

The content of the following submission on XML file is incorporated herein by reference in its entirety: a computer readable form (CRF) of the Sequence Listing (file name: IEC240075PCT-seql.xml, date recorded: Jul. 30, 2024, size: 180,487 bytes).

### TECHNICAL FIELD

This disclosure relates to chimeric cytokine receptors, engineered immune effector cells comprising same, and methods of use thereof.

### BACKGROUND

The chimeric antigen receptor (CAR)-based therapies are a rapidly emerging form of cancer treatment, and have resulted in remarkable responses in diseases such as refractory lymphoid malignancies. However, the therapeutic effects of CAR-T cells targeting various malignancies have not yet resulted in significant clinical benefits. Although inefficient tumor trafficking and various immunosuppressive mechanisms can impede CAR-T cell effector responses, the signals delivered by the current CAR constructs may still be insufficient to fully activate antitumor T cell functions. Optimal T cell activation and proliferation require multiple signals, including T cell receptor (TCR) engagement (signal 1), co-stimulation (signal 2), and cytokine engagement (signal 3) (see, e.g., Kershaw M. H. et al. Gene-engineered T cells for cancer therapy. Nat Rev Cancer. 2013; 13:525-541). Cytokines can deliver proliferative signals to T cells, however, approaches to provide cytokine engagement signals have met limitations and thus solutions are needed to improve CAR T cells' efficacy and safety. Therefore, there exists a need for improved CAR therapies with chimeric cytokine receptors.

### SUMMARY

The present disclosure relates chimeric cytokine receptors (CCRs) and engineered immune cells (e.g., CAR T cells) that express such CCRs.

In one aspect, the disclosure is related to a chimeric cytokine receptor comprising: (a) a first extracellular antigen binding domain, (b) a first transmembrane domain, (c) a Janus Kinase (JAK)-binding domain comprising a TPOR BOX1 motif, wherein optionally the JAK-binding domain does not comprise a TPOR BOX2 motif, and (d) a cytokine receptor intracellular domain.

In some embodiments, the first transmembrane domain is a TPOR transmembrane domain, a CD28 transmembrane domain, a CD8 transmembrane domain or a DAP10 transmembrane domain.

In some embodiments, the JAK-binding domain comprises an amino acid sequence set forth in SEQ ID NO: 8 or an amino acid sequence that is at least 80%, 85%, 90%, 95% or 99% identical to the amino acid sequence set forth in SEQ ID NO: 8.

In one aspect, the disclosure is related to a chimeric cytokine receptor comprising: (a) a first extracellular antigen binding domain, (b) a first transmembrane domain, wherein the first transmembrane domain is selected from a CD28 transmembrane domain, a CD8 transmembrane domain and a DAP10 transmembrane domain, (c) a JAK-binding domain, and (d) a cytokine receptor intracellular domain.

In some embodiments, the JAK-binding domain comprises a TPOR BOX1 motif and a TPOR BOX2 motif.

In some embodiments, the JAK-binding domain comprises an amino acid sequence set forth in SEQ ID NO: 7 or an amino acid sequence that is at least 80%, 85%, 90%, 95% or 99% identical to the amino acid sequence set forth in SEQ ID NO: 7.

In some embodiments, the JAK-binding domain comprises a TPOR BOX1 motif, and wherein the JAK-binding domain does not comprise a TPOR BOX2 motif.

In some embodiments, the JAK-binding domain comprises an amino acid sequence set forth in SEQ ID NO: 8 or an amino acid sequence that is at least 80%, 85%, 90%, 95% or 99% identical to the amino acid sequence set forth in SEQ ID NO: 8.

In some embodiments, the cytokine receptor intracellular domain comprises a STAT-binding domain, wherein optionally the STAT-binding domain comprises an IL-21R intracellular domain and/or an IL-15Rβ intracellular domain.

In some embodiments, the IL-21R intracellular domain comprises an amino acid sequence set forth in SEQ ID NO: 20 or 21 or an amino acid sequence that is at least 80%, 85%, 90%, 95% or 99% identical to the amino acid sequence set forth in SEQ ID NO: 20 or 21.

In some embodiments, the IL-15Rβ intracellular domain comprises an amino acid sequence set forth in any one of SEQ ID NOs: 10-18 or an amino acid sequence that is at least 80%, 85%, 90%, 95% or 99% identical to the amino acid sequence set forth in any one of SEQ ID NOs: 10-18.

In some embodiments, the first extracellular antigen binding domain binds to an antigen expressed on the surface of a tumor cell.

In some embodiments, the first extracellular antigen binding domain is derived from NKG2D, truncated NKG2D, a NKG2D extracellular domain, an antibody or antigen binding fragment thereof targeting NKG2D ligands, TIGIT or SIRP-α, or a variant thereof.

In some embodiments, the first extracellular antigen binding domain is derived from NKG2D or truncated NKG2D, or a variant thereof, wherein optionally the first extracellular antigen binding domain is derived from the extracellular domain (ECD) of NKG2D or truncated NKG2D, and wherein optionally the first extracellular antigen binding domain comprises the amino acid sequence of SEQ ID NO: 6, or an amino acid sequence having at least 75%, 80%, 85%, 90%, 91%, 92%, 93%, 94%, 95%, 96%, 97%, 98%, or 99% identity to SEQ ID NO: 6.

In some embodiments, the chimeric cytokine receptor comprises the amino acid sequence of SEQ ID NO: 103, 105, 107, 109 or 111, or an amino acid sequence having at least 75%, 80%, 85%, 90%, 91%, 92%, 93%, 94%, 95%, 96%, 97%, 98%, or 99% identity to SEQ ID NO: 103, 105, 107, 109 or 111.

In one aspect, the disclosure is related to a cell comprising any one of the chimeric cytokine receptors described herein.

In some embodiments, the cell further comprises a functional exogenous receptor.

In some embodiments, the functional exogenous receptor comprising: (a) a second extracellular antigen binding domain, (b) a second transmembrane domain, and (c) an intracellular signaling domain.

In some embodiments, the functional exogenous receptor is a T cell receptor (TCR), a chimeric antigen receptor (CAR), a chimeric TCR (cTCR), or a T cell antigen coupler (TAC)-like chimeric receptor.

In some embodiments, the functional exogenous receptor is a CAR, wherein optionally the CAR is a single CAR, dual CAR, tandem CAR or split CAR.

In some embodiments, the CAR binds to a tumor-associated antigen, wherein optionally the tumor-associated antigen is selected from the group consisting of CD19, AFP, BCMA, NY-ESO-1, VEGFR2, MAGE-A3, CD20, CD22, CD30, CD33, GUCY2C, MUC17, FGFR2, CLL1, CD38, CEA, CS1, CD138, CD123/IL3Rα, c-Met, gp100, MUC1, IGF-I receptor, EpCAM, CEA, EGFR (such as EGFRvIII), GD2, HER2, IGF1R, mesothelin, PSMA, ROR1, WT1, Glypican 3 (GPC3), Guanylate cyclase 2C (GCC), DLL3, Claudin18.2, Claudin6, Glycolipid F77, PD-L1, PD-L2, and other tumor antigens with clinical significance, and combinations thereof.

In some embodiments, the CAR comprises a single chain variable fragment (scFv) comprising a heavy chain complementarity determining region 1 (CDR1), CDR2, CDR3, a light chain complementarity determining region 1 (CDR1), CDR2, and CDR3, having the amino acid sequences of:
(1) SEQ ID NOs: 38, 39, 40, 41, 42, 43, respectively;
(2) SEQ ID NOs: 44, 45, 46, 47, 48, 49, respectively;
(3) SEQ ID NOs: 50, 51, 52, 53, 54, 55, respectively; or
(4) SEQ ID NOs: 97, 98, 99, 100, 101, 102, respectively.

In some embodiments, the CAR comprises a heavy chain variable region (VH) comprising CDR1, CDR2, and CDR3 and a light chain variable region (VL) comprising CDR1, CDR2, and CDR3, having the amino acid sequences of:
(1) the VH sequence is SEQ ID NO: 72 and the VL sequence is SEQ ID NO: 73;
(2) the VH sequence is SEQ ID NO: 74, and the VL sequence is SEQ ID NO: 75;
(3) the VH sequence is SEQ ID NO: 76, and the VL sequence is SEQ ID NO: 77; or
(4) the VH sequence is SEQ ID NO: 94, and the VL sequence is SEQ ID NO: 95.

In some embodiments, the CAR comprises a V_{H}H antibody moiety that having the amino acid sequences of:
a CDR1 comprising the amino acid sequence of SEQ ID NO: 56, a CDR2 comprising the amino acid sequence of SEQ ID NO: 57, and a CDR3 comprising the amino acid sequence of SEQ ID NO: 58;
a CDR1 comprising the amino acid sequence of SEQ ID NO: 59, a CDR2 comprising the amino acid sequence of SEQ ID NO: 60, and a CDR3 comprising the amino acid sequence of SEQ ID NO: 61;
a CDR1 comprising the amino acid sequence of SEQ ID NO: 62, a CDR2 comprising the amino acid sequence of SEQ ID NO: 63, and a CDR3 comprising the amino acid sequence of SEQ ID NO: 64; or a CDR1 comprising the amino acid sequence of SEQ ID NO: 65, a CDR2 comprising the amino acid sequence of SEQ ID NO: 66, and a CDR3 comprising the amino acid sequence of SEQ ID NO: 67.

In some embodiments, the CAR comprises a V_{H}H antibody moiety comprising the amino acid sequences of any one of SEQ ID NOs: 68-71 or an amino acid sequence having at least 90%, 95%, or 99% identity to the amino acid sequence of any one of SEQ ID NOs: 68-71.

In some embodiments, the intracellular signaling domain comprises a primary intracellular signaling domain of a cell, wherein optionally the primary intracellular signaling domain is from CD3ζ.

In some embodiments, the intracellular signaling domain comprises a co-stimulatory signaling domain, wherein optionally the co-stimulatory signaling domain is derived from a co-stimulatory molecule selected from the group consisting of CD27, CD28, CD137, OX40, CD30, CD40, CD3, LFA-1, ICOS, CD2, CD7, LIGHT, NKG2C, B7-H3, ligands of CD83 and combinations thereof.

In some embodiments, the CAR comprises an amino acid sequence set forth in any one of SEQ ID NOs: 4, 78-83 and 91, or an amino acid sequence having at least 90%, 95%, or 99% identity to the amino acid sequence set forth in any one of SEQ ID NOs: 4, 78-83 and 91.

In some embodiments, the chimeric cytokine receptor and the functional exogenous receptor are linked to each other via a peptide linker.

In some embodiments, the peptide linker is a self-cleaving peptide linker, wherein optionally the self-cleaving peptide linker is a 2A self-cleaving peptide, wherein optionally the 2A self-cleaving peptide is selected from a group consisting of F2A, E2A, P2A, T2A, and variants thereof.

In some embodiments, the cell comprises the amino acid sequence set forth in any one of SEQ ID NOs: 22-37, 92 and 93, or an amino acid sequence that is at least 80%, 85%, 90%, 95% or 99% identical to the amino acid sequence set forth in any one of SEQ ID NOs: 22-37, 92 and 93.

In some embodiments, the cell is an immune cell.

In some embodiments, the immune cell is selected from a group consisting of T cell, NK cell, NKT, peripheral blood mononuclear cell (PBMC), hematopoietic stem cell, pluripotent stem cell, an embryonic stem cell, a macrophage, a monocyte, a neutrophil, an eosinophil and a combination thereof.

In some embodiments, the T cell is a αβT cell, γδT cell or a combination of αβT and γδT cell.

In some embodiments, the T cell, upon activation, exhibits increased expression of pSTAT3 and pSTAT5.

In one aspect, the disclosure is related to a nucleic acid comprising a nucleic acid sequence encoding any one of the chimeric cytokine receptors described herein.

In some embodiments, the nucleic acid further comprises a nucleic acid sequence encoding a functional exogenous receptor comprising: (a) a second extracellular antigen binding domain, (b) a second transmembrane domain, and (c) an intracellular signaling domain.

In some embodiments, the functional exogenous receptor is a T cell receptor (TCR), a chimeric antigen receptor (CAR), a chimeric TCR (cTCR), or a T cell antigen coupler (TAC)-like chimeric receptor.

In some embodiments, the CAR binds to a tumor-associated antigen, wherein optionally the tumor-associated antigen is selected from the group consisting of CD19, AFP, BCMA, NY-ESO-1, VEGFR2, MAGE-A3, CD20, CD22, CD30, CD33, GUCY2C, MUC17, FGFR2, CLL1, CD38, CEA, CS1, CD138, CD123/IL3Rα, c-Met, gp100, MUC1, IGF-I receptor, EpCAM, CEA, EGFR (such as EGFRvIII), GD2, HER2, IGFIR, mesothelin, PSMA, ROR1, WT1, Glypican 3 (GPC3), Guanylate cyclase 2C (GCC), DLL3, Claudin18.2, Claudin6, Glycolipid F77, PD-L1, PD-L2, and other tumor antigens with clinical significance, and combinations thereof.

In some embodiments, the CAR comprises a single chain variable fragment (scFv) comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 38, 39, 40, respectively, and the selected VL CDRs 1, 2, 3 amino acid sequences are set forth in SEQ ID NOs: 41, 42, 43, respectively;
(2) the selected VH CDRs 1, 2, 3 amino acid sequences are set forth in SEQ ID NOs: 44, 45, 46, respectively, and the selected VL CDRs 1, 2, 3 amino acid sequences are set forth in SEQ ID NOs: 47, 48, 49, respectively;
(3) the selected VH CDRs 1, 2, 3 amino acid sequences are set forth in SEQ ID NOs: 50, 51, 52, respectively, and the selected VL CDRs 1, 2, 3 amino acid sequences are set forth in SEQ ID NOs: 53, 54, 55, respectively; and
(4) the selected VH CDRs 1, 2, 3 amino acid sequences are set forth in SEQ ID NOs: 97, 98, 99, respectively, and the selected VL CDRs 1, 2, 3 amino acid sequences are set forth in SEQ ID NOs: 100, 101, 102, respectively.

In some embodiments, the CAR comprises a heavy chain variable region (VH) comprising an amino acid sequence that is at least 80% identical to a selected VH sequence, and a light chain variable region (VL) comprising an amino acid sequence that is at least 80% identical to a selected VL sequence, wherein the selected VH sequence and the selected VL sequence are one of the following:
(1) the selected VH sequence is SEQ ID NO: 72, and the selected VL sequence is SEQ ID NO: 73;
(2) the selected VH sequence is SEQ ID NO: 74, and the selected VL sequence is SEQ ID NO: 75;
(3) the selected VH sequence is SEQ ID NO: 76, and the selected VL sequence is SEQ ID NO: 77; and
(4) the selected VH sequence is SEQ ID NO: 94, and the selected VL sequence is SEQ ID NO: 95.

In some embodiments, the CAR comprises a V_{H}H antibody moiety that having the amino acid sequences of:
a CDR1 comprising the amino acid sequence of SEQ ID NO: 56, a CDR2 comprising the amino acid sequence of SEQ ID NO: 57, and a CDR3 comprising the amino acid sequence of SEQ ID NO: 58;
a CDR1 comprising the amino acid sequence of SEQ ID NO: 59, a CDR2 comprising the amino acid sequence of SEQ ID NO: 60, and a CDR3 comprising the amino acid sequence of SEQ ID NO: 61;
a CDR1 comprising the amino acid sequence of SEQ ID NO: 62, a CDR2 comprising the amino acid sequence of SEQ ID NO: 63, and a CDR3 comprising the amino acid sequence of SEQ ID NO: 64; or
a CDR1 comprising the amino acid sequence of SEQ ID NO: 65, a CDR2 comprising the amino acid sequence of SEQ ID NO: 66, and a CDR3 comprising the amino acid sequence of SEQ ID NO: 67.

In some embodiments, the CAR comprises a V_{H}H antibody moiety comprising an amino acid sequence of any one of SEQ ID NOs: 68-71 or an amino acid sequence having at least 90%, 95%, or 99% identity to the amino acid sequence of any one of SEQ ID NOs: 68-71.

In some embodiments, the intracellular signaling domain further comprises a co-stimulatory signaling domain.

In some embodiments, the CAR comprises an amino acid sequence set forth in any one of SEQ ID NOs: 4, 78-83 and 91, or an amino acid sequence having at least 90%, 95%, or 99% identity to the amino acid sequence set forth in any one of SEQ ID NOs: 4, 78-83 and 91.

In some embodiments, the chimeric cytokine receptor nucleic acid sequence and the nucleic acid sequence encoding the functional exogenous receptor are linked to each other via a third nucleic acid sequence encoding a peptide linker.

In some embodiments, the nucleic acid comprises a nucleic acid sequence encoding an amino acid sequence set forth in any one of SEQ ID NOs: 22-37, 92 and 93, or an amino acid sequence that is at least 80%, 85%, 90%, 95% or 99% identical to the amino acid sequence set forth in any one of SEQ ID NOs: 22-37, 92 and 93.

In one aspect, the disclosure is related to a vector comprising any one of the nucleic acids described herein.

In one aspect, the disclosure is related to a pharmaceutical composition, comprising any one of the chimeric cytokine receptors described herein, any one of the cells described herein, or any one of the nucleic acids described herein and a pharmaceutically acceptable carrier.

In one aspect, the disclosure is related to a method of treating a disease or disorder in a subject, comprising administering to the subject an effective amount of the pharmaceutical composition described herein.

In some embodiments, the disease or disorder is a cancer, an inflammatory or autoimmune disease.

In some embodiments, the cancer is solid cancer or hematologic cancer.

In some embodiments, the cancer is liver cancer, gastric cancer, colon cancer, lymphoma, acute myeloid leukemia (AML) or chronic myelogenous leukemia (CML).

Also described herein is an antibody or antigen-binding fragment thereof, comprising:
a single chain variable fragment (scFv) comprising a heavy chain complementarity determining region 1 (CDR1), CDR2, CDR3, a light chain complementarity determining region 1 (CDR1), CDR2, and CDR3, having the amino acid sequences of:
(1) SEQ ID NOs: 38, 39, 40, 41, 42, 43, respectively;
(2) SEQ ID NOs: 44, 45, 46, 47, 48, 49, respectively;
(3) SEQ ID NOs: 50, 51, 52, 53, 54, 55, respectively; or
(4) SEQ ID NOs: 97, 98, 99, 100, 101, 102, respectively.

Also described herein is an antibody or antigen-binding fragment thereof, comprising: a heavy chain variable region (VH) comprising CDR1, CDR2, and CDR3 and a light chain variable region (VL) comprising CDR1, CDR2, and CDR3, having the amino acid sequences of:
(1) the VH sequence is SEQ ID NO: 72 and the VL sequence is SEQ ID NO: 73;
(2) the VH sequence is SEQ ID NO: 74, and the VL sequence is SEQ ID NO: 75;
(3) the VH sequence is SEQ ID NO: 76, and the VL sequence is SEQ ID NO: 77; or
(4) the VH sequence is SEQ ID NO: 94, and the VL sequence is SEQ ID NO: 95.

Also described herein is a V_{H}H antibody moiety having the amino acid sequences of:
(1) a CDR1 comprising the amino acid sequence of SEQ ID NO: 56, a CDR2 comprising the amino acid sequence of SEQ ID NO: 57, and a CDR3 comprising the amino acid sequence of SEQ ID NO: 58;
(2) a CDR1 comprising the amino acid sequence of SEQ ID NO: 59, a CDR2 comprising the amino acid sequence of SEQ ID NO: 60, and a CDR3 comprising the amino acid sequence of SEQ ID NO: 61;
(3) a CDR1 comprising the amino acid sequence of SEQ ID NO: 62, a CDR2 comprising the amino acid sequence of SEQ ID NO: 63, and a CDR3 comprising the amino acid sequence of SEQ ID NO: 64; or
(4) a CDR1 comprising the amino acid sequence of SEQ ID NO: 65, a CDR2 comprising the amino acid sequence of SEQ ID NO: 66, and a CDR3 comprising the amino acid sequence of SEQ ID NO: 67.

In some embodiments, the V_{H}H antibody moiety comprises the amino acid sequences of any one of SEQ ID NOs: 68-71 or an amino acid sequence having at least 90%, 95%, or 99% identity to the amino acid sequence of any one of SEQ ID NOs: 68-71.

As used herein, the terms “intracellular domain,” “intracellular region” and “cytoplasmic region” are used interchangeably herein to refer to the portion of a receptor that is inside the cell. The intracellular domain can be the entire portion of a receptor that is inside the cell, or just a part thereof (e.g., at least 50%, 60%, 70%, 80%, 85%, 90%, 95%, 96%, 97%, 98%, or 99% of the entire portion). The intracellular domain can be derived from the intracellular domain of a wild-type membrane protein (e.g., a receptor) or a functional variant thereof. The intracellular domain may have one or more mutations, including e.g., insertions, deletions, and/or substitutions. The intracellular domain may be part of a chimeric antigen receptor (CAR).

The intracellular domain may be part of a chimeric cytokine receptor. The intracellular domain may be a cytokine receptor intracellular domain. The cytokine receptor intracellular domain may comprise a STAT-binding domain. The STAT-binding domain may comprise an IL-21R intracellular domain and/or an IL-15Rβ intracellular domain.

As used herein, the terms “extracellular domain” or “extracellular region” are used interchangeably herein to refer to the portion of a receptor that is outside the cell membrane. The extracellular domain can be entire portion of a receptor that is outside the cell membrane, or just a part thereof (e.g., at least 50%, 60%, 70%, 80%, 85%, 90%, 95%, 96%, 97%, 98%, or 99% of the entire portion). The extracellular domain can be derived from the extracellular domain of a wild-type receptor or a functional variant thereof. The extracellular domain can have one or more mutations, including e.g., insertions, deletions, or substitutions.

As used herein, the term “IL-21R intracellular domain” refers to the intracellular domain derived from IL-21R or a functional variant thereof. The IL-21R intracellular domain may have one or more mutations, including e.g., insertions, deletions, and/or substitutions. The IL-21R intracellular domain intracellular domain may comprise a sequence of SEQ ID NO: 20 or 21, or a sequence that is at least 80%, 85%, 90%, or 95% to SEQ ID NO: 20 or 21.

As used herein, the term “IL-15Rβ intracellular domain” refers to the intracellular domain derived from IL-15Rβ or a functional variant thereof. The IL-15Rβ intracellular domain may have one or more mutations, including e.g., insertions, deletions, and/or substitutions. The IL-15Rβ intracellular domain may comprise a sequence of any one of SEQ ID NOs: 10-18 or a sequence that is at least 80%, 85%, 90%, or 95% identical to any one of SEQ ID NOs: 10-18.

As used herein, the term “co-stimulatory signaling domain” refers to the portion of the receptor that includes one or more domains (e.g., intracellular domains) from co-stimulatory proteins for the receptor to persist after activation. The co-stimulatory signaling domain can be derived from the intracellular domains of a wild-type co-stimulatory protein or a functional variant thereof. The co-stimulatory signaling domain may have one or more mutations, including e.g., insertions, deletions, and/or substitutions.

As used herein, the term “primary intracellular signaling domain” refers to cytoplasmic signaling sequence that acts in a stimulatory manner to induce immune effector functions. The primary intracellular signaling domain may contain a signaling motif known as immunoreceptor tyrosine-based activation motif, or ITAM. An “ITAM,” as used herein, is a conserved protein motif that is generally present in the tail portion of signaling molecules expressed in many immune cells.

The motif can comprise two repeats of the amino acid sequence YxxL/I separated by 6-8 amino acids, wherein each x is independently any amino acid, producing the conserved motif YxxL/Ix (6-8) YxxL/I. ITAMs within signaling molecules are important for signal transduction within the cell, which is mediated at least in part by phosphorylation of tyrosine residues in the ITAM following activation of the signaling molecule. ITAMs can also function as docking sites for other proteins involved in signaling pathways. Exemplary ITAM-containing primary cytoplasmic signaling sequences include those derived from CD3ζ, FcR gamma (FCER1G), FcR beta (Fc Epsilon Rib), CD3 gamma, CD3 delta, CD3 epsilon, CD5, CD22, CD79a, CD79b, and CD66d. The primary intracellular signaling domain may derive from CD3ζ. The intracellular signaling domain may consist of a CD3 cytoplasmic signaling domain. The primary intracellular signaling domain may be derived from a cytoplasmic signaling domain of wildtype CD32. The primary intracellular signaling domain may be a functional variant of the cytoplasmic signaling domain of CD35 containing one or more mutations, such as Q65K.

As used herein, the terms “transmembrane domain” or “transmembrane region” are used interchangeably herein to refer to the portion of a membrane protein (e.g., a receptor) that is embedded in the cell membrane. The transmembrane domain can be entire portion of the protein that is embedded in the cell membrane, or just a part thereof (e.g., at least 50%, 60%, 70%, 80%, 85%, 90%, 95%, 96%, 97%, 98%, or 99% of the entire portion). The transmembrane domain may be derived from the transmembrane domain of a wild-type receptor or a functional variant thereof. The transmembrane domain may have one or more mutations, including e.g., insertions, deletions, and/or substitutions.

As used herein, the term “CD8 transmembrane domain” refers to the transmembrane domain derived from CD8 or a functional variant thereof. The CD8 transmembrane domain may have one or more mutations, including e.g., insertions, deletions, and/or substitutions. The CD8 transmembrane domain can comprise a sequence of SEQ ID NO: 2 or a sequence that is at least 80%, 85%, 90%, or 95% identical to SEQ ID NO: 2.

As used herein, the term “CD28 transmembrane domain” refers to the transmembrane domain derived from CD28 or a functional variant thereof. The CD28 transmembrane domain may have one or more mutations, including e.g., insertions, deletions, and/or substitutions. The CD28 transmembrane domain can comprise a sequence of SEQ ID NO: 3 or a sequence that is at least 80%, 85%, 90%, or 95% identical to SEQ ID NO: 3.

As used herein, the term “DAP10 transmembrane domain” refers to the transmembrane domain derived from DAP10 or a functional variant thereof. The DAP10 transmembrane domain may have one or more mutations, including e.g., insertions, deletions, and/or substitutions. The DAP10 transmembrane domain can comprise a sequence of SEQ ID NO: 96 or a sequence that is at least 80%, 85%, 90%, or 95% identical to SEQ ID NO: 96.

As used herein, the terms “hinge domain” or “hinge region” are used interchangeably herein to refer to the portion of a membrane protein (e.g., a receptor) that connects the transmarine region and the extracellular domain. The hinge region can be part of an extracellular region. The hinge region can be derived from the hinge region of a wild-type receptor or a functional variant thereof. The hinge region may have one or more mutations, including e.g., insertions, deletions, and/or substitutions.

As used herein, the term “CD8 hinge region” refers to the hinge region derived from CD8 or a functional variant thereof. The CD8 hinge region can have one or more mutations, including e.g., insertions, deletions, and/or substitutions. The CD8 hinge region and transmembrane domain may comprise a sequence of SEQ ID NO: 86 or a sequence that is at least 80%, 85%, 90%, or 95% identical to SEQ ID NO: 86.

As used herein, the term “Janus Kinase (JAK)-binding domain” refers to a domain that binds to a member of the JAK family. The Janus kinase/signal transducer and activator of transcription (JAK/STAT) signaling pathway is regarded as one of the central communication nodes in the cell function. More than 50 cytokines and growth factors have been identified in the JAK/STAT signaling pathway, such as hormones, interferons (IFN), interleukins (ILs), and colony-stimulating factors (CSFs). The JAK/STAT signaling pathway is evolutionarily conserved. It is composed of ligand-receptor complexes, JAKs, and STATs. There are 4 members in the JAK family: JAK1, JAK2, JAK3, and TYK2.

As used herein, the term “STAT-binding domain” refers to a domain that binds to a member of the STAT family. The STAT family comprises seven members: STAT1, STAT2, STAT3, STAT4, STAT5a, STAT5b, and STAT6. The STAT-binding domain may be an IL-15Rβ STAT-binding domain and include an intracellular domain of IL-15R. The STAT-binding domain may be an IL-21R STAT-binding domain and include an intracellular domain of IL-21R.

As used herein, the “NKG2D ligand binding domain” refers to a domain that binds to a ligand of NKG2D. The NKG2D ligand binding domain may be derived from NKG2D, truncated NKG2D, a NKG2D extracellular domain, or an antibody or antigen binding fragment thereof targeting NKG2D ligands. The NKG2D ligand binding domain may be an NKG2D extracellular domain. The NKG2D ligand binding domain may be a truncated NKG2D sequence. The NKG2D ligand binding domain may have the amino acid sequence of SEQ ID NO: 6, or an amino acid sequence having at least 75%, 80%, 85%, 90%, 91%, 92%, 93%, 94%, 95%, 96%, 97%, 98%, or 99% identity to SEQ ID NO: 6.

The term “complementarity determining region” or “CDR,” as used herein, refers to the sequences of amino acids within antibody variable domains which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The extent of CDRs and the framework region can be precisely identified using methodology known in the art, for example, by the Kabat definition. See, e.g., Kabat, E. A., et al. (1991) Sequences of Proteins of Immunological Interest, Fifth Edition, U.S. Department of Health and Human Services, NIH Publication No. 91-3242. One or more positions according to the Kabat numbering may not be occupied in the actual sequence, or the actual sequence may contain more amino acid residues than the number allowed for by the Kabat numbering. See, e.g., Deschacht et al., 2010. J Immunol 184:5696-704 for an exemplary numbering for V_{H}H domains according to Kabat. If not described to the contrary, CDRs are according to Kabat numbering.

As used herein, a “vector” is any construct capable of delivering one or more polynucleotides of interest to a host cell when the vector is introduced to the host cell. An “expression vector” is capable of delivering and expressing the one or more polynucleotides of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

As used herein, the term “chimeric cytokine receptor” or “CCR” refers to a molecule that comprises an extracellular antigen binding domain, a transmembrane domain and a cytokine receptor intracellular domain. The chimeric cytokine receptor may further comprise a JAK-binding domain. The cytokine receptor intracellular domain may derive from different cytokine receptors or are involved in different cytokine pathways.

As used herein, the term “chimeric antigen receptor” or “CAR” as used herein refers to genetically engineered receptors, which can be used to graft one or more antigen specificity onto immune effector cells, such as T cells. Some CARs are also known as “artificial T-cell receptors,” “chimeric T cell receptors,” or “chimeric immune receptors.” The CAR may comprise an extracellular ligand binding domain or an extracellular antigen binding domain specific for one or more antigens (such as tumor antigens), a transmembrane domain, and an intracellular signaling domain. “CAR-T cell” refers to a T cell that expresses a CAR. “CAR-NK cell” refers to an NK cell that expresses a CAR.

As used herein, the term “cancer” or “cancer cell” refers to the cells dividing in an uncontrolled manner, e.g., forming the solid tumors or the excessive tumor cells in blood. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. The cancer cells can form the solid tumors or the excessive tumor cells in blood (e.g., hematologic cancer). Alternatively or additionally it can include all types of cancerous growths or oncogenic processes, metastatic tissues or malignantly transformed cells, tissues, or organs, irrespective of histopathologic type or stage of invasiveness. Examples of solid tumors include malignancies, e.g., sarcomas, adenocarcinomas, and carcinomas, of the various organ systems, such as those affecting liver, lung, breast, lymphoid, gastrointestinal (e.g., colon), genitourinary tract (e.g., renal, urothelial cells), prostate and pharynx. Adenocarcinomas include malignancies such as most colon cancers, rectal cancer, renal-cell carcinoma, liver cancer, non-small cell carcinoma of the lung, cancer of the small intestine and cancer of the esophagus. Examples of cancers that can be treated by the methods described herein include e.g., bone cancer, pancreatic cancer, skin cancer (e.g., melanoma), cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin Disease, non-Hodgkin lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia (AML), chronic myeloid leukemia, acute lymphoblastic leukemia, chronic lymphocytic leukemia, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, and/or T cell lymphoma.

As used herein, the term “antigen binding domain” refers to a domain that is capable of specifically binding to an antigen. The antigen binding domain may be a portion of a full-length antibody, an antigen binding fragment, or the extracellular domain of a receptor. An antigen binding fragment may comprise at least one variable domain (e.g., a variable domain of a heavy chain, single domain antibody or V_{H}H). Non-limiting examples of antibody fragments include, e.g., Fab, Fab′, F(ab′)₂, and Fv fragments. An extracellular antigen binding domain may be an extracellular cytokine binding domain (e.g., a NKG2D ligand binding domain).

As used herein, the term “derived from” when made in reference to a domain described herein refers to a domain that is obtained from the relevant functional portion of a protein (e.g., by recombinant expression or de novo synthesis). The term encompasses domains with naturally occurring sequences and sequences with mutations. A domain derived from a particular protein can have a sequence that is at least 80%, 85%, 90%, 95%, 98%, 99%, or 100% identical to the relevant functional portion of the particular protein. A domain derived from a particular protein can be from a natural or a synthetic source. For example, a primary intracellular signaling domain derived from CD3ζ can have a sequence that is identical to the intracellular signaling domain of CD3ζ, or at least 80%, 85%, 90%, 95%, 98% or 99% identical to the sequence of the intracellular signaling domain of CD3ζ.

As used herein, the terms “subject” and “patient” are used interchangeably throughout the specification and describe an animal, human or non-human, to whom treatment according to the methods of the present disclosure is provided. Veterinary and non-veterinary applications are contemplated by the present disclosure. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the disclosure will be apparent from the following detailed description and figures, and from the claims.

### DESCRIPTION OF DRAWINGS

FIG. 1 shows a schematic of a CAR armored with NKG2D, truncated NKG2D or mutated NKG2D chimeric cytokine receptor.

FIG. 2 shows a schematic of a CAR armored with chimeric cytokine receptor which includes a binding domain targeting NKG2D ligands.

FIGS. 3A-3C show exemplary constructs of CARs armored with NKG2D chimeric cytokine receptor using different transmembrane domain and these designs efficacy in vitro. FIG. 3A shows exemplary constructs of CARs armored with NKG2D chimeric cytokine receptor using different transmembrane domain. FIG. 3B shows long-term cytotoxicity of NKG2D chimeric cytokine receptors using different transmembrane domain armored anti-GPC3 CAR T cells co-culture with Huh7 cells. FIG. 3C shows T cells proliferation in long-term co-cultures of NKG2D chimeric cytokine receptors using different transmembrane domain armored anti-GPC3 CAR T cells with Huh7 cells.

FIGS. 4A-4C show exemplary constructs of CARs armored with NKG2D chimeric cytokine receptor exploring the role of JAK-binding domain TPOR BOX2 and these designs efficacy in vitro. FIG. 4A shows exemplary constructs of CARs armored with NKG2D chimeric cytokine receptor exploring the role of JAK-binding domain TPOR BOX2. FIG. 4B shows long-term cytotoxicity of anti-GPC3 CAR T cells co-culture with Huh7 cells. FIG. 4C shows T cells proliferation in long-term co-cultures of anti-GPC3 CAR T cells with Huh7 cells.

FIGS. 5A-5C show exemplary constructs of CARs armored with NKG2D chimeric cytokine receptor using TPOR or IL-15Rβ JAK-binding domain and these designs efficacy in vitro. FIG. 5A shows exemplary constructs of CARs armored with NKG2D chimeric cytokine receptor using TPOR or IL-15Rβ JAK-binding domain. FIG. 5B shows long-term cytotoxicity of anti-GPC3 CAR T cells co-culture with Huh7 cells. FIG. 5C shows T cells proliferation in long-term co-cultures of anti-GPC3 CAR T cells with Huh7 cells.

FIGS. 6A-6C show exemplary constructs of CARs armored with NKG2D chimeric cytokine receptor using CD28 transmembrane domain and TPOR BOX1 and these designs efficacy in vitro. FIG. 6A shows exemplary constructs of CARs armored with NKG2D chimeric cytokine receptor using CD28 transmembrane domain and TPOR BOX1. FIG. 6B shows long-term cytotoxicity of anti-GPC3 CAR T cells co-culture with Huh7 cells. FIG. 6C shows T cells proliferation in long-term co-cultures of anti-GPC3 CAR T cells with Huh7 cells.

FIGS. 7A-7E show exemplary constructs of CARs armored with NKG2D chimeric cytokine receptor using two functionally synergistic STAT-binding domains and these designs efficacy in vitro. FIG. 7A shows exemplary constructs of CARs armored with NKG2D chimeric cytokine receptor using two functionally synergistic STAT-binding domains. FIGS. 7B and 7D show long-term cytotoxicity of anti-GPC3 CAR T cells co-culture with Huh7 cells. FIGS. 7C and 7E show T cells proliferation in long-term co-cultures of anti-GPC3 CAR T cells with Huh7 cells.

FIGS. 8A-8E show the exemplary construct of the functional sites of IL-15Rβ STAT-binding domain and CARs armored with NKG2D chimeric cytokine receptor identifying the functional sites of IL-15Rβ STAT-binding domain efficacy in vitro. FIG. 8A shows the exemplary construct of the functional sites of IL-15Rβ STAT-binding domain. FIGS. 8B and 8D show long-term cytotoxicity of anti-GPC3 CAR T cells co-culture with Huh7 cells. FIG. 8C and FIG. 8E show T cells proliferation in long-term co-cultures of anti-GPC3 CAR T cells with Huh7 cells.

FIGS. 9A-9H show the in vivo efficacy of anti-GPC3 CAR-T cells and anti-GPC3 CAR-T cells armored with NKG2D chimeric cytokine receptor in Huh7 xenograft model. FIG. 9I shows the mice plasma IFN-γ cytokine section of anti-GPC3 CAR-T and anti-GPC3 CAR-T cells armored with NKG2D chimeric cytokine receptor.

FIGS. 10A-10B show the efficacy and proliferation of anti-GPC3 AZ-CAR armored with NKG2D chimeric cytokine receptor using B6 or B6M2 in vitro. FIG. 10A shows long-term cytotoxicity of anti-GPC3 CAR T cells co-culture with Huh7 cells. FIG. 10B shows T cells proliferation in long-term co-cultures of anti-GPC3 CAR T cells with Huh7 cells.

FIGS. 11A-11F show selected sequences listed in the present application.

### DETAILED DESCRIPTION

The present disclosure relates to engineered immune cells (e.g., CAR T cells) that express chimeric cytokine receptor. More specifically, modified chimeric cytokine receptors were developed and the role of each element of the chimeric cytokine receptor was assessed carefully to adapt to various complex tumor environments.

### Chimeric Cytokine Receptors (CCRs)

A chimeric cytokine receptor (CCR) is a molecule which comprises a cytokine receptor intracellular domain and a heterologous ligand-binding extracellular domain (e.g., an extracellular antigen binding domain). The cytokine receptor intracellular domain may derive from different cytokine receptors or are involved in different cytokine pathways. The chimeric cytokine receptor described herein may comprise an intracellular domain that causes “cytokine-type” cell signaling when the extracellular domain binds its ligand.

Successful adoptive T cell therapy requires a robust expansion and persistence of administered T cells, and the environmental signals received by the T cell contribute heavily to these behaviors. In preclinical models, chimeric antigen receptor (CAR) T cell therapy can be improved by supplementing the therapy with gamma chain cytokines, which are soluble factors that promote T cell growth and survival. However, systemic administration of cytokines to patients is not a therapeutically viable solution, as clinical trials have shown that such intervention leads to toxic side-effects (see, e.g., Jeught V. et al. (2014) Oncotarget 6:1359-81). In order to confer the benefits of cytokine supplementation to CAR T cell therapy without incurring systemic toxicity, chimeric cytokine receptors can be engineered to provide T cell-intrinsic constitutive interleukin signaling. Chimeric cytokine receptors can recapitulate the signaling events of specific cytokines.

In one aspect, the disclosure provides a chimeric cytokine receptor comprising: (a) an extracellular antigen binding domain, (b) a transmembrane domain, (c) a Janus Kinase (JAK)-binding domain comprising a TPOR BOX1 motif, wherein optionally the JAK-binding domain does not comprise a TPOR BOX2 motif, and (d) a cytokine receptor intracellular domain.

In one aspect, the disclosure provides a chimeric cytokine receptor comprising: (a) an extracellular antigen binding domain, (b) a transmembrane domain, (c) a Janus Kinase (JAK)-binding domain comprising a TPOR BOX1 motif, wherein optionally the JAK-binding domain does not comprise a TPOR BOX2 motif, and (d) a cytokine receptor intracellular domain.

In one aspect, the disclosure provides a chimeric cytokine receptor comprising: (a) a NKG2D ligand binding domain, (b) a transmembrane domain, (c) and a cytokine receptor intracellular domain.

In one aspect, the disclosure provides a chimeric cytokine receptor comprising: (a) a extracellular antigen binding domain, (b) a transmembrane domain, wherein the transmembrane domain is selected from a CD28 transmembrane domain, a CD8 transmembrane domain and a DAP10 transmembrane domain (c) a JAK-binding domain, and (d) a cytokine receptor intracellular domain.

NKG2D is an activating receptor that is mostly expressed on cells of the cytotoxic arm of the immune system. Ligands of NKG2D are normally of low abundance, but can be induced in virtually any cell in response to stressors, such as infection and oncogenic transformation. Engagement of NKG2D stimulates the production of cytokines and cytotoxic molecules and traditionally this receptor is viewed as a molecule that mediates direct responses against cellular threats. During NK cell development, engagement of NKG2D also has a long-term impact on the expression of NK cell receptors and their responsiveness to extracellular cues, suggesting a role in NK cell education. Upon chronic NKG2D engagement, both NK and T cells show reduced responsiveness of a number of activating receptors, demonstrating a role of NKG2D in induction of peripheral tolerance.

The NKG2D ligand binding domain may be derived from NKG2D, truncated NKG2D, a NKG2D extracellular domain, or an antibody or antigen binding fragment thereof targeting NKG2D ligands. The NKG2D ligand binding domain may be an NKG2D extracellular domain. The NKG2D ligand binding domain may be a truncated NKG2D sequence. The NKG2D ligand binding domain may have the amino acid sequence of SEQ ID NO: 6, or an amino acid sequence having at least 75%, 80%, 85%, 90%, 91%, 92%, 93%, 94%, 95%, 96%, 97%, 98%, or 99% identity to SEQ ID NO: 6.

The transmembrane domain can be any suitable transmembrane domain known in the art. The transmembrane domain is a structural component, consisting of a hydrophobic alpha helix that spans the cell membrane. It anchors the engineered receptor (e.g., the chimeric cytokine receptor) to the plasma membrane, bridging the extracellular hinge and antigen binding domains with the intracellular signaling region. This domain is essential for the stability of the receptor as a whole. Generally, the transmembrane domain from the most membrane-proximal component of the intracellular domain is used, but different transmembrane domains may result in different receptor stability. The transmembrane domain may be a transmembrane domain of CD137 (4-1BB), an alpha chain of a T cell receptor, a beta chain of a T cell receptor, CD3 epsilon, CD4, CD5, CD8, CD8 alpha, CD9, CD16, CD19, CD22, CD28, CD33, CD37, CD45, CD64, CD80, CD86, CD134, CD137, CD154, or a zeta chain of a T cell receptor, or any combination thereof.

The transmembrane domain may be a TPOR transmembrane domain, a CD28 transmembrane domain, a CD8 transmembrane domain or a DAP10 transmembrane domain. The TPOR transmembrane domain may comprise an amino acid sequence set forth in SEQ ID NO: 9 or an amino acid sequence that is at least 80%, 85%, 90%, 95% or 99% identical to the amino acid sequence set forth in SEQ ID NO: 9. The CD28 transmembrane domain may comprise an amino acid sequence set forth in SEQ ID NO: 3 or an amino acid sequence that is at least 80%, 85%, 90%, 95% or 99% identical to the amino acid sequence set forth in SEQ ID NO: 3. The CD8 transmembrane domain may comprise an amino acid sequence set forth in SEQ ID NO: 2 or an amino acid sequence that is at least 80%, 85%, 90%, 95% or 99% identical to the amino acid sequence set forth in SEQ ID NO: 2. The DAP10 transmembrane domain may comprise an amino acid sequence set forth in SEQ ID NO: 96 or an amino acid sequence that is at least 80%, 85%, 90%, 95% or 99% identical to the amino acid sequence set forth in SEQ ID NO: 96.

The JAK-binding domain may comprise a TPOR BOX1 motif and a TPOR BOX2 motif. The JAK-binding domain may comprise an amino acid sequence set forth in SEQ ID NO: 7 or an amino acid sequence that is at least 80%, 85%, 90%, 95% or 99% identical to the amino acid sequence set forth in SEQ ID NO: 7.

The JAK-binding domain may comprise a TPOR BOX1 motif. The JAK-binding domain may not comprise a TPOR BOX2 motif. The JAK-binding domain may comprise an amino acid sequence set forth in SEQ ID NO: 8 or an amino acid sequence that is at least 80%, 85%, 90%, 95% or 99% identical to the amino acid sequence set forth in SEQ ID NO: 8.

The cytokine receptor intracellular domain may comprise a STAT-binding domain. The STAT-binding domain may comprise an IL-21R intracellular domain and/or an IL-15Rβ intracellular domain.

The amino acid sequence of the wild-type IL-21R intracellular domain is shown in SEQ ID NO: 88. The full-length amin acid sequence of IL-21R is shown in SEQ ID NO: 89. The IL-21R intracellular domain shown in SEQ ID NO: 88 is amino acids 254-538 of the full-length amino acid sequence of IL-21R. The IL-21R intracellular domain shown in SEQ ID NO: 88 is amino acids 254-538 of SEQ ID NO: 89. The IL-21R intracellular domain of the chimeric cytokine receptor described herein may have one or more mutations, including e.g., insertions, deletions, and/or substitutions. The IL-21R intracellular domain may comprise a sequence of SEQ ID NO: 20 or 21 or a sequence that is at least 80%, 85%, 90%, or 95% identical to SEQ ID NO: 20 or 21. The amino acid sequence set forth in SEQ ID NO: 20 includes a portion of the wild-type IL-21R intracellular domain (amino acids 504-538 of the full-length IL-21R amino acid sequence, i.e., positions 504-538 of SEQ ID NO: 89). The amino acid sequence set forth in SEQ ID NO: 21 includes a portion of the IL-21R intracellular domain with a Y519F mutation (position 519 of SEQ ID NO: 89). This mutation is a functional site mutation that reduces the signaling transduction function of the IL-21R intracellular domain. The IL-21R intracellular domain may have a Y519F mutation.

The amino acid sequence of the wild-type IL-15Rβ intracellular domain is shown in SEQ ID NO: 87. The full-length amin acid sequence of IL-15Rβ is shown in SEQ ID NO: 90. The IL-15Rβ intracellular domain shown in SEQ ID NO: 87 is amino acids 266-551 of the full-length amin acid sequence of IL-15Rβ. The IL-15Rβ intracellular domain shown in SEQ ID NO: 87 is amino acids 266-551 of SEQ ID NO: 90. The IL-15Rβ intracellular domain of the chimeric cytokine receptor described herein may have one or more mutations, including e.g., insertions, deletions, and/or substitutions. The IL-15Rβ intracellular domain may comprise a sequence of any one of SEQ ID NOs: 10-18 or a sequence that is at least 80%, 85%, 90%, or 95% identical to any one of SEQ ID NOs: 10-18. The amino acid sequence set forth in SEQ ID NO: 10 includes a portion of the sequence of the wild-type IL-15Rβ intracellular domain (amino acids 345-431 and 521-551 of the full-length IL-15Rβ amino acid sequence, i.e., positions 345-431 and 521-551 of SEQ ID NO: 90). The amino acid sequence set forth in SEQ ID NO: 11 includes a portion of the sequence of the wild-type IL-15Rβ intracellular domain (amino acids 345-431 and 521-551 of the full-length IL-15Rβ amino acid sequence, i.e., positions 345-431 and 521-551 of SEQ ID NO: 90) with a Y364F mutation (position 364 of SEQ ID NO: 90). The amino acid sequence set forth in SEQ ID NO: 12 includes a portion of the sequence of the wild-type IL-15Rβ intracellular domain (amino acids 345-431 and 521-551 of the full-length IL-15Rβ amino acid sequence, i.e., positions 345-431 and 521-551 of SEQ ID NO: 90) with the Y364F, Y381F, Y384F and Y387F mutations (positions 364, 381, 384, and 387 of SEQ ID NO: 90). The amino acid sequence set forth in SEQ ID NO: 13 includes a portion of the sequence of the wild-type IL-15Rβ intracellular domain (amino acids 345-431 and 521-551 of the full-length IL-15Rβ amino acid sequence, i.e., positions 345-431 and 521-551 of SEQ ID NO: 90) with a Y418F mutation (position 418 of SEQ ID NO: 90). The amino acid sequence set forth in SEQ ID NO: 14 includes a portion of the sequence of the wild-type IL-15Rβ intracellular domain (amino acids 345-431 and 521-551 of the full-length IL-15Rβ amino acid sequence, i.e., positions 345-431 and 521-551 of SEQ ID NO: 90) with a Y536F mutation (position 536 of SEQ ID NO: 90). The amino acid sequence set forth in SEQ ID NO: 15 includes a portion of the sequence of the wild-type IL-15Rβ intracellular domain (amino acids 345-431 and 521-551 of the full-length IL-15Rβ amino acid sequence, i.e., positions 345-431 and 521-551 of SEQ ID NO: 90) with the Y418F and Y536F mutations (positions 418 and 536 of SEQ ID NO: 90). The amino acid sequence set forth in SEQ ID NO: 16 includes a portion of the sequence of the wild-type IL-15Rβ intracellular domain (amino acids 345-431 and 521-551 of the full-length IL-15Rβ amino acid sequence, i.e., positions 345-431 and 521-551 of SEQ ID NO: 90) with the Y364F, Y381F, Y384F, Y387F, Y418F and Y536F mutations (positions 364, 381, 384, 387, 418, and 536 of SEQ ID NO: 90). The amino acid sequence set forth in SEQ ID NO: 17 includes the sequence of the truncated IL-15Rβ intracellular domain (amino acids 345-431 of the full-length IL-15Rβ amino acid sequence, i.e., positions 345-431 of SEQ ID NO: 90). The amino acid sequence set forth in SEQ ID NO: 18 includes the sequence of the truncated IL-15Rβ intracellular domain (amino acids 345-404 of the full-length IL-15Rβ amino acid sequence, i.e., positions 345-404 of SEQ ID NO: 90). The IL-15Rβ intracellular domain may have one or more mutations selected from Y364F, Y381F, Y384F, Y387F, Y418F and Y536F of the full-length IL-15Rβ amino acid sequence. The IL-15Rβ intracellular domain may have one or more mutations selected from Y364F, Y381F, Y384F, Y387F, Y418F and Y536F of SEQ ID NO: 90.

The JAK-binding domain may comprise an IL-15Rβ BOX1 motif and an IL-15Rβ BOX2 motif. The JAK-binding domain may comprise an amino acid sequence set forth in SEQ ID NO: 19 or an amino acid sequence that is at least 80%, 85%, 90%, 95% or 99% identical to the amino acid sequence set forth in SEQ ID NO: 19.

The extracellular antigen-binding domain of the chimeric cytokine receptors (CCRs) described herein can be any suitable antigen-binding fragments known in the art. The extracellular antigen-binding domain is exposed to the outside of the cell, in the extracellular domain portion of the CCR. It interacts with potential target molecules and is responsible for targeting the CCR-expressing cell to any cell expressing a matching molecule. The antigen-binding domain is typically derived from the variable regions of a monoclonal antibody linked together as a single-chain variable fragment (scFv). An scFv is a chimeric protein made up of the light (VL) and heavy (VH) chains of immunoglobulins, connected with a short linker peptide. The antigen binding domain may comprise one or more (e.g., 1, 2, 3, 4, 5, or 6) VH-VL pairs. The antigen binding domain may comprise one or more (e.g., 1, 2, 3, 4, 5, or 6) heavy chain single variable domains (V_{H}Hs). The V_{H}Hs may be connected with a linker peptide (e.g., a flexible linker). The linker peptide between the two V_{H}Hs includes hydrophilic residues with stretches of glycine and serine in it for flexibility as well as stretches of glutamate and lysine for added solubility.

The extracellular antigen-binding domain may be a Fab fragment, F(ab′) fragment, F(ab′)₂ fragment or Fv fragment. The antibody may be any one of various types of antibodies capable of binding antigen-specifically, briefly, having antigen-specific binding ability. For example, the antibody may be one in which one light chain and one heavy chain are bonded with each other, or one in which two light chains and two heavy chains are bonded with each other. For example, when two light chains and two heavy chains are bonded with each other, the antibody may be one in which a first unit including a first light chain and a first heavy chain bonded with each other and a second unit including a second light chain and a second heavy chain bonded with each other are combined with each other. The bond may be a disulfide bond. The two units may be the same as or different from each other. The first unit may include the first light chain and the first heavy chain and the second unit including the second light chain and the second heavy chain are the same as or different from each other. As such, an antibody prepared to recognize two different antigens by the first unit and the second unit, respectively, is typically referred to as a “bispecific antibody.” The antibody may be one in which three or more of the above-described units are combined with one another. The antigen-binding fragment of the present disclosure may be derived from various types of antibodies.

The extracellular antigen binding domain of the CCR may bind to an antigen on a tumor cell.

The extracellular antigen binding domain may be derived from NKG2D, truncated NKG2D, a NKG2D extracellular domain, an antibody or antigen binding fragment thereof targeting NKG2D ligands, TIGIT or SIRP-α, or a variant thereof. The extracellular antigen binding domain of the CCR may bind to an NKG2D ligand on a tumor cell.

The extracellular antigen binding domain may be derived from NKG2D or truncated NKG2D, or a variant thereof. The extracellular antigen binding domain may be derived from the extracellular domain (ECD) of NKG2D or truncated NKG2D. The extracellular antigen binding domain may comprise the amino acid sequence of SEQ ID NO: 6, or an amino acid sequence having at least 75%, 80%, 85%, 90%, 91%, 92%, 93%, 94%, 95%, 96%, 97%, 98%, or 99% identity to SEQ ID NO: 6.

The cell may comprise a NKG2D chimeric cytokine receptor molecule comprising from the N-terminus to the C-terminus: a NKG2D ECD (e.g., SEQ ID NO: 6 or a sequence that is at least 80%, 85%, 90%, or 95% identical to SEQ ID NO: 6), a TPOR transmembrane (TM) (e.g., SEQ ID NO: 9 or a sequence that is at least 80%, 85%, 90%, or 95% identical to SEQ ID NO: 9) and TPOR BOX1-BOX2 (e.g., SEQ ID NO: 7 or a sequence that is at least 80%, 85%, 90%, or 95% identical to SEQ ID NO: 7), a IL-15Rβ ICD (e.g., SEQ ID NO: 10 or a sequence that is at least 80%, 85%, 90%, or 95% identical to SEQ ID NO: 10) and a IL-21R ICD (e.g., SEQ ID NO: 20 or a sequence that is at least 80%, 85%, 90%, or 95% identical to SEQ ID NO: 20). The cell may comprise an amino acid sequence set forth in SEQ ID NO: 103, or an amino acid sequence that is at least 80%, 85%, 90%, 95% or 99% identical to the amino acid sequence set forth in SEQ ID NO: 103. The NKG2D chimeric cytokine receptor molecule may further comprise a signal peptide (e.g., SEQ ID NO: 1 or a sequence that is at least 80%, 85%, 90%, or 95% identical to SEQ ID NO: 1). The cell may comprise an amino acid sequence set forth in SEQ ID NO: 104, or an amino acid sequence that is at least 80%, 85%, 90%, 95% or 99% identical to the amino acid sequence set forth in SEQ ID NO: 104.

The cell may comprise a NKG2D chimeric cytokine receptor molecule comprising from the N-terminus to the C-terminus: a NKG2D ECD (e.g., SEQ ID NO: 6 or a sequence that is at least 80%, 85%, 90%, or 95% identical to SEQ ID NO: 6), a CD8 hinge (e.g., SEQ ID NO: 86 or a sequence that is at least 80%, 85%, 90%, or 95% identical to SEQ ID NO: 86), a CD8TM (e.g., SEQ ID NO: 2 or a sequence that is at least 80%, 85%, 90%, or 95% identical to SEQ ID NO: 2), a TPOR BOX1-BOX2 (e.g., SEQ ID NO: 7 or a sequence that is at least 80%, 85%, 90%, or 95% identical to SEQ ID NO: 7), a IL-15Rβ ICD (e.g., SEQ ID NO: 10 or a sequence that is at least 80%, 85%, 90%, or 95% identical to SEQ ID NO: 10) and a IL-21R ICD (e.g., SEQ ID NO: 20 or a sequence that is at least 80%, 85%, 90%, or 95% identical to SEQ ID NO: 20). The cell may comprise an amino acid sequence set forth in SEQ ID NO: 113, or an amino acid sequence that is at least 80%, 85%, 90%, 95% or 99% identical to the amino acid sequence set forth in SEQ ID NO: 113. The NKG2D chimeric cytokine receptor molecule may further comprise a signal peptide (e.g., SEQ ID NO: 1 or a sequence that is at least 80%, 85%, 90%, or 95% identical to SEQ ID NO: 1). The cell may comprise an amino acid sequence set forth in SEQ ID NO: 114, or an amino acid sequence that is at least 80%, 85%, 90%, 95% or 99% identical to the amino acid sequence set forth in SEQ ID NO: 114.

The cell may comprise a NKG2D chimeric cytokine receptor molecule comprising from the N-terminus to the C-terminus: a NKG2D ECD (e.g., SEQ ID NO: 6 or a sequence that is at least 80%, 85%, 90%, or 95% identical to SEQ ID NO: 6), a CD28 TM (e.g., SEQ ID NO: 3 or a sequence that is at least 80%, 85%, 90%, or 95% identical to SEQ ID NO: 3), a TPOR BOX1-BOX2 (e.g., SEQ ID NO: 7 or a sequence that is at least 80%, 85%, 90%, or 95% identical to SEQ ID NO: 7), a IL-15Rβ ICD (e.g., SEQ ID NO: 10 or a sequence that is at least 80%, 85%, 90%, or 95% identical to SEQ ID NO: 10) and a IL-21R ICD (e.g., SEQ ID NO: 20 or a sequence that is at least 80%, 85%, 90%, or 95% identical to SEQ ID NO: 20). The cell may comprise an amino acid sequence set forth in SEQ ID NO: 111, or an amino acid sequence that is at least 80%, 85%, 90%, 95% or 99% identical to the amino acid sequence set forth in SEQ ID NO: 111. The NKG2D chimeric cytokine receptor molecule may further comprise a signal peptide (e.g., SEQ ID NO: 1 or a sequence that is at least 80%, 85%, 90%, or 95% identical to SEQ ID NO: 1). The cell may comprise an amino acid sequence set forth in SEQ ID NO: 112, or an amino acid sequence that is at least 80%, 85%, 90%, 95% or 99% identical to the amino acid sequence set forth in SEQ ID NO: 112.

The cell may comprise a NKG2D chimeric cytokine receptor molecule comprising from the N-terminus to the C-terminus: a NKG2D ECD (e.g., SEQ ID NO: 6 or a sequence that is at least 80%, 85%, 90%, or 95% identical to SEQ ID NO: 6), a TPOR TM (e.g., SEQ ID NO: 9 or a sequence that is at least 80%, 85%, 90%, or 95% identical to SEQ ID NO: 9) and TPOR-BOX1 (e.g., SEQ ID NO: 8 or a sequence that is at least 80%, 85%, 90%, or 95% identical to SEQ ID NO: 8), a IL-15Rβ ICD (e.g., SEQ ID NO: 10 or a sequence that is at least 80%, 85%, 90%, or 95% identical to SEQ ID NO: 10) and a IL-21R ICD (e.g., SEQ ID NO: 20 or a sequence that is at least 80%, 85%, 90%, or 95% identical to SEQ ID NO: 20). The cell may comprise an amino acid sequence set forth in SEQ ID NO: 115, or an amino acid sequence that is at least 80%, 85%, 90%, 95% or 99% identical to the amino acid sequence set forth in SEQ ID NO: 115. The NKG2D chimeric cytokine receptor molecule may further comprise a signal peptide (e.g., SEQ ID NO: 1 or a sequence that is at least 80%, 85%, 90%, or 95% identical to SEQ ID NO: 1). The cell may comprise an amino acid sequence set forth in SEQ ID NO: 116, or an amino acid sequence that is at least 80%, 85%, 90%, 95% or 99% identical to the amino acid sequence set forth in SEQ ID NO: 116.

The cell may comprise a NKG2D chimeric cytokine receptor molecule comprising from the N-terminus to the C-terminus: a NKG2D ECD (e.g., SEQ ID NO: 6 or a sequence that is at least 80%, 85%, 90%, or 95% identical to SEQ ID NO: 6), a TPOR TM (e.g., SEQ ID NO: 9 or a sequence that is at least 80%, 85%, 90%, or 95% identical to SEQ ID NO: 9) and IL-15Rβ BOX1-BOX2 (e.g., SEQ ID NO: 19 or a sequence that is at least 80%, 85%, 90%, or 95% identical to SEQ ID NO: 19), a IL-15Rβ ICD (e.g., SEQ ID NO: 10 or a sequence that is at least 80%, 85%, 90%, or 95% identical to SEQ ID NO: 10) and a IL-21R ICD (e.g., SEQ ID NO: 20 or a sequence that is at least 80%, 85%, 90%, or 95% identical to SEQ ID NO: 20). The cell may comprise an amino acid sequence set forth in SEQ ID NO: 117, or an amino acid sequence that is at least 80%, 85%, 90%, 95% or 99% identical to the amino acid sequence set forth in SEQ ID NO: 117. The NKG2D chimeric cytokine receptor molecule may further comprise a signal peptide (e.g., SEQ ID NO: 1 or a sequence that is at least 80%, 85%, 90%, or 95% identical to SEQ ID NO: 1). The cell may comprise an amino acid sequence set forth in SEQ ID NO: 118, or an amino acid sequence that is at least 80%, 85%, 90%, 95% or 99% identical to the amino acid sequence set forth in SEQ ID NO: 118.

The cell may comprise a NKG2D chimeric cytokine receptor molecule comprising from the N-terminus to the C-terminus: a NKG2D ECD (e.g., SEQ ID NO: 6 or a sequence that is at least 80%, 85%, 90%, or 95% identical to SEQ ID NO: 6), a CD28 TM (e.g., SEQ ID NO: 3 or a sequence that is at least 80%, 85%, 90%, or 95% identical to SEQ ID NO: 3), a TPOR-BOX1 (e.g., SEQ ID NO: 8 or a sequence that is at least 80%, 85%, 90%, or 95% identical to SEQ ID NO: 8), a IL-15Rβ ICD (e.g., SEQ ID NO: 10 or a sequence that is at least 80%, 85%, 90%, or 95% identical to SEQ ID NO: 10) and a IL-21R ICD (e.g., SEQ ID NO: 20 or a sequence that is at least 80%, 85%, 90%, or 95% identical to SEQ ID NO: 20). The cell may comprise an amino acid sequence set forth in SEQ ID NO: 107, or an amino acid sequence that is at least 80%, 85%, 90%, 95% or 99% identical to the amino acid sequence set forth in SEQ ID NO: 107. The NKG2D chimeric cytokine receptor molecule may further comprise a signal peptide (e.g., SEQ ID NO: 1 or a sequence that is at least 80%, 85%, 90%, or 95% identical to SEQ ID NO: 1). The cell may comprise an amino acid sequence set forth in SEQ ID NO: 108, or an amino acid sequence that is at least 80%, 85%, 90%, 95% or 99% identical to the amino acid sequence set forth in SEQ ID NO: 108.

The cell may comprise a NKG2D chimeric cytokine receptor molecule comprising from the N-terminus to the C-terminus: a NKG2D ECD (e.g., SEQ ID NO: 6 or a sequence that is at least 80%, 85%, 90%, or 95% identical to SEQ ID NO: 6), a TPOR TM (e.g., SEQ ID NO: 9 or a sequence that is at least 80%, 85%, 90%, or 95% identical to SEQ ID NO: 9) and TPOR BOX1-BOX2 (e.g., SEQ ID NO: 7 or a sequence that is at least 80%, 85%, 90%, or 95% identical to SEQ ID NO: 7), a IL-21R ICD (e.g., SEQ ID NO: 20 or a sequence that is at least 80%, 85%, 90%, or 95% identical to SEQ ID NO: 20) and a IL-15Rβ ICD (e.g., SEQ ID NO: 10 or a sequence that is at least 80%, 85%, 90%, or 95% identical to SEQ ID NO: 10). The cell may comprise an amino acid sequence set forth in SEQ ID NO: 119, or an amino acid sequence that is at least 80%, 85%, 90%, 95% or 99% identical to the amino acid sequence set forth in SEQ ID NO: 119. The NKG2D chimeric cytokine receptor molecule may further comprise a signal peptide (e.g., SEQ ID NO: 1 or a sequence that is at least 80%, 85%, 90%, or 95% identical to SEQ ID NO: 1). The cell may comprise an amino acid sequence set forth in SEQ ID NO: 120, or an amino acid sequence that is at least 80%, 85%, 90%, 95% or 99% identical to the amino acid sequence set forth in SEQ ID NO: 120.

The cell may comprise a NKG2D chimeric cytokine receptor molecule comprising from the N-terminus to the C-terminus: a NKG2D ECD (e.g., SEQ ID NO: 6 or a sequence that is at least 80%, 85%, 90%, or 95% identical to SEQ ID NO: 6), a TPOR TM (e.g., SEQ ID NO: 9 or a sequence that is at least 80%, 85%, 90%, or 95% identical to SEQ ID NO: 9) and TPOR BOX1-BOX2 (e.g., SEQ ID NO: 7 or a sequence that is at least 80%, 85%, 90%, or 95% identical to SEQ ID NO: 7), a IL-15Rβ truncated1 345-431 ICD (e.g., SEQ ID NO: 17 or a sequence that is at least 80%, 85%, 90%, or 95% identical to SEQ ID NO: 17) and a IL-21R ICD (e.g., SEQ ID NO: 20 or a sequence that is at least 80%, 85%, 90%, or 95% identical to SEQ ID NO: 20). The cell may comprise an amino acid sequence set forth in SEQ ID NO: 109, or an amino acid sequence that is at least 80%, 85%, 90%, 95% or 99% identical to the amino acid sequence set forth in SEQ ID NO: 109. The NKG2D chimeric cytokine receptor molecule may forth in SEQ ID NO: 110, or an amino acid sequence that is at least 80%, 85%, 90%, 95% or 99% identical to the amino acid sequence set forth in SEQ ID NO: 110.

The cell may comprise a NKG2D chimeric cytokine receptor molecule comprising from the N-terminus to the C-terminus: a NKG2D ECD (e.g., SEQ ID NO: 6 or a sequence that is at least 80%, 85%, 90%, or 95% identical to SEQ ID NO: 6), a TPOR TM (e.g., SEQ ID NO: 9 or a sequence that is at least 80%, 85%, 90%, or 95% identical to SEQ ID NO: 9) and TPOR BOX1-BOX2 (e.g., SEQ ID NO: 7 or a sequence that is at least 80%, 85%, 90%, or 95% identical to SEQ ID NO: 7), a IL-15Rβ truncated2 345-404 ICD (e.g., SEQ ID NO: 18 or a sequence that is at least 80%, 85%, 90%, or 95% identical to SEQ ID NO: 18) and a IL-21R ICD (e.g., SEQ ID NO: 20 or a sequence that is at least 80%, 85%, 90%, or 95% identical to SEQ ID NO: 20). The cell may comprise an amino acid sequence set forth in SEQ ID NO: 105, or an amino acid sequence that is at least 80%, 85%, 90%, 95% or 99% identical to the amino acid sequence set forth in SEQ ID NO: 105. The NKG2D chimeric cytokine receptor molecule may further comprise a signal peptide (e.g., SEQ ID NO: 1 or a sequence that is at least 80%, 85%, 90%, or 95% identical to SEQ ID NO: 1). The cell may comprise an amino acid sequence set forth in SEQ ID NO: 106, or an amino acid sequence that is at least 80%, 85%, 90%, 95% or 99% identical to the amino acid sequence set forth in SEQ ID NO: 106.

The cell may comprise a NKG2D chimeric cytokine receptor molecule comprising from the N-terminus to the C-terminus: a NKG2D ECD (e.g., SEQ ID NO: 6 or a sequence that is at least 80%, 85%, 90%, or 95% identical to SEQ ID NO: 6), a TPOR TM (e.g., SEQ ID NO: 9 or a sequence that is at least 80%, 85%, 90%, or 95% identical to SEQ ID NO: 9) and TPOR BOX1-BOX2 (e.g., SEQ ID NO: 7 or a sequence that is at least 80%, 85%, 90%, or 95% identical to SEQ ID NO: 7), a IL-15Rβ ICD (e.g., SEQ ID NO: 10 or a sequence that is at least 80%, 85%, 90%, or 95% identical to SEQ ID NO: 10) and a IL-21R ICD with Y519F mutation (e.g., SEQ ID NO: 21 or a sequence that is at least 80%, 85%, 90%, or 95% identical to SEQ ID NO: 21). The cell may comprise an amino acid sequence set forth in SEQ ID NO: 121, or an amino acid sequence that is at least 80%, 85%, 90%, 95% or 99% identical to the amino acid sequence set forth in SEQ ID NO: 121. The NKG2D chimeric cytokine receptor molecule may forth in SEQ ID NO: 122, or an amino acid sequence that is at least 80%, 85%, 90%, 95% or 99% identical to the amino acid sequence set forth in SEQ ID NO: 122.

The cell may comprise a NKG2D chimeric cytokine receptor molecule comprising from the N-terminus to the C-terminus: a NKG2D ECD (e.g., SEQ ID NO: 6 or a sequence that is at least 80%, 85%, 90%, or 95% identical to SEQ ID NO: 6), a TPOR TM (e.g., SEQ ID NO: 9 or a sequence that is at least 80%, 85%, 90%, or 95% identical to SEQ ID NO: 9) and TPOR BOX1-BOX2 (e.g., SEQ ID NO: 7 or a sequence that is at least 80%, 85%, 90%, or 95% identical to SEQ ID NO: 7), a IL-15Rβ ICD with Y364F mutation (e.g., SEQ ID NO: 11 or a sequence that is at least 80%, 85%, 90%, or 95% identical to SEQ ID NO: 11) and a IL-21R ICD (e.g., SEQ ID NO: 20 or a sequence that is at least 80%, 85%, 90%, or 95% identical to SEQ ID NO: 20). The cell may comprise an amino acid sequence set forth in SEQ ID NO: 123, or an amino acid sequence that is at least 80%, 85%, 90%, 95% or 99% identical to the amino acid sequence set forth in SEQ ID NO: 123. The NKG2D chimeric cytokine receptor molecule may further comprise a signal peptide (e.g., SEQ ID NO: 1 or a sequence that is at least 80%, 85%, 90%, or 95% identical to SEQ ID NO: 1). The cell may comprise an amino acid sequence set forth in SEQ ID NO: 124, or an amino acid sequence that is at least 80%, 85%, 90%, 95% or 99% identical to the amino acid sequence set forth in SEQ ID NO: 124.

The cell may comprise a NKG2D chimeric cytokine receptor molecule comprising from the N-terminus to the C-terminus: a NKG2D ECD (e.g., SEQ ID NO: 6 or a sequence that is at least 80%, 85%, 90%, or 95% identical to SEQ ID NO: 6), a TPOR TM (e.g., SEQ ID NO: 9 or a sequence that is at least 80%, 85%, 90%, or 95% identical to SEQ ID NO: 9) and TPOR BOX1-BOX2 (e.g., SEQ ID NO: 7 or a sequence that is at least 80%, 85%, 90%, or 95% identical to SEQ ID NO: 7), a IL-15Rβ ICD with Y364F, Y381F, Y384F, Y387F mutation (e.g., SEQ ID NO: 12 or a sequence that is at least 80%, 85%, 90%, or 95% identical to SEQ ID NO: 12) and a IL-21R ICD (e.g., SEQ ID NO: 20 or a sequence that is at least 80%, 85%, 90%, or 95% identical to SEQ ID NO: 20). The cell may comprise an amino acid sequence set forth in SEQ ID NO: 125, or an amino acid sequence that is at least 80%, 85%, 90%, 95% or 99% identical to the amino acid sequence set forth in SEQ ID NO: 125. The NKG2D chimeric cytokine receptor molecule may further comprise a signal peptide (e.g., SEQ ID NO: 1 or a sequence that is at least 80%, 85%, 90%, or 95% identical to SEQ ID NO: 1). The cell may comprise an amino acid sequence set forth in SEQ ID NO: 126, or an amino acid sequence that is at least 80%, 85%, 90%, 95% or 99% identical to the amino acid sequence set forth in SEQ ID NO: 126.

The cell may comprise a NKG2D chimeric cytokine receptor molecule comprising from the N-terminus to the C-terminus: a NKG2D ECD (e.g., SEQ ID NO: 6 or a sequence that is at least 80%, 85%, 90%, or 95% identical to SEQ ID NO: 6), a TPOR TM (e.g., SEQ ID NO: 9 or a sequence that is at least 80%, 85%, 90%, or 95% identical to SEQ ID NO: 9) and TPOR BOX1-BOX2 (e.g., SEQ ID NO: 7 or a sequence that is at least 80%, 85%, 90%, or 95% identical to SEQ ID NO: 7), a IL-15Rβ ICD with Y418F mutation (e.g., SEQ ID NO: 13 or a sequence that is at least 80%, 85%, 90%, or 95% identical to SEQ ID NO: 13) and a IL-21R ICD (e.g., SEQ ID NO: 20 or a sequence that is at least 80%, 85%, 90%, or 95% identical to SEQ ID NO: 20). The cell may comprise an amino acid sequence set forth in SEQ ID NO: 127, or an amino acid sequence that is at least 80%, 85%, 90%, 95% or 99% identical to the amino acid sequence set forth in SEQ ID NO: 127. The NKG2D chimeric cytokine receptor molecule may further comprise a signal peptide (e.g., SEQ ID NO: 1 or a sequence that is at least 80%, 85%, 90%, or 95% identical to SEQ ID NO: 1). The cell may comprise an amino acid sequence set forth in SEQ ID NO: 128, or an amino acid sequence that is at least 80%, 85%, 90%, 95% or 99% identical to the amino acid sequence set forth in SEQ ID NO: 128.

The cell may comprise a NKG2D chimeric cytokine receptor molecule comprising from the N-terminus to the C-terminus: a NKG2D ECD (e.g., SEQ ID NO: 6 or a sequence that is at least 80%, 85%, 90%, or 95% identical to SEQ ID NO: 6), a TPOR TM (e.g., SEQ ID NO: 9 or a sequence that is at least 80%, 85%, 90%, or 95% identical to SEQ ID NO: 9) and TPOR BOX1-BOX2 (e.g., SEQ ID NO: 7 or a sequence that is at least 80%, 85%, 90%, or 95% identical to SEQ ID NO: 7), a IL-15Rβ ICD with Y536F mutation (e.g., SEQ ID NO: 14 or a sequence that is at least 80%, 85%, 90%, or 95% identical to SEQ ID NO: 14) and a IL-21R ICD (e.g., SEQ ID NO: 20 or a sequence that is at least 80%, 85%, 90%, or 95% identical to SEQ ID NO: 20). The cell may comprise an amino acid sequence set forth in SEQ ID NO: 129, or an amino acid sequence that is at least 80%, 85%, 90%, 95% or 99% identical to the amino acid sequence set forth in SEQ ID NO: 129. The NKG2D chimeric cytokine receptor molecule may forth in SEQ ID NO: 130, or an amino acid sequence that is at least 80%, 85%, 90%, 95% or 99% identical to the amino acid sequence set forth in SEQ ID NO: 130.

The cell may comprise a NKG2D chimeric cytokine receptor molecule comprising from the N-terminus to the C-terminus: a NKG2D ECD (e.g., SEQ ID NO: 6 or a sequence that is at least 80%, 85%, 90%, or 95% identical to SEQ ID NO: 6), a TPOR TM (e.g., SEQ ID NO: 9 or a sequence that is at least 80%, 85%, 90%, or 95% identical to SEQ ID NO: 9) and TPOR BOX1-BOX2 (e.g., SEQ ID NO: 7 or a sequence that is at least 80%, 85%, 90%, or 95% identical to SEQ ID NO: 7), a IL-15Rβ ICD with Y418F, Y536F mutation (e.g., SEQ ID NO: 15 or a sequence that is at least 80%, 85%, 90%, or 95% identical to SEQ ID NO: 15) and a IL-21R ICD (e.g., SEQ ID NO: 20 or a sequence that is at least 80%, 85%, 90%, or 95% identical to SEQ ID NO: 20). The cell may comprise an amino acid sequence set forth in SEQ ID NO: 131, or an amino acid sequence that is at least 80%, 85%, 90%, 95% or 99% identical to the amino acid sequence set forth in SEQ ID NO: 131. The NKG2D chimeric cytokine receptor molecule may further comprise a signal peptide (e.g., SEQ ID NO: 1 or a sequence that is at least 80%, 85%, 90%, or 95% identical to SEQ ID NO: 1). The cell may comprise an amino acid sequence set forth in SEQ ID NO: 132, or an amino acid sequence that is at least 80%, 85%, 90%, 95% or 99% identical to the amino acid sequence set forth in SEQ ID NO: 132.

The cell may comprise a NKG2D chimeric cytokine receptor molecule comprising from the N-terminus to the C-terminus: a NKG2D ECD (e.g., SEQ ID NO: 6 or a sequence that is at least 80%, 85%, 90%, or 95% identical to SEQ ID NO: 6), a TPOR TM (e.g., SEQ ID NO: 9 or a sequence that is at least 80%, 85%, 90%, or 95% identical to SEQ ID NO: 9) and TPOR BOX1-BOX2 (e.g., SEQ ID NO: 7 or a sequence that is at least 80%, 85%, 90%, or 95% identical to SEQ ID NO: 7), a IL-15Rβ ICD with Y364F, Y381F, Y384F, Y387F, Y418F, Y536F mutation (e.g., SEQ ID NO: 16 or a sequence that is at least 80%, 85%, 90%, or 95% identical to SEQ ID NO: 16) and a IL-21R ICD (e.g., SEQ ID NO: 20 or a sequence that is at least 80%, 85%, 90%, or 95% identical to SEQ ID NO: 20). The cell may comprise an amino acid sequence set forth in SEQ ID NO: 133, or an amino acid sequence that is at least 80%, 85%, 90%, 95% or 99% identical to the amino acid sequence set forth in SEQ ID NO: 133. The NKG2D chimeric cytokine receptor molecule may further comprise a signal peptide (e.g., SEQ ID NO: 1 or a sequence that is at least 80%, 85%, 90%, or 95% identical to SEQ ID NO: 1). The cell may comprise an amino acid sequence set forth in SEQ ID NO: 134, or an amino acid sequence that is at least 80%, 85%, 90%, 95% or 99% identical to the amino acid sequence set forth in SEQ ID NO: 134.

The extracellular antigen binding domain of the chimeric cytokine receptor provided herein may be monospecific. The extracellular antigen binding domain of the chimeric cytokine receptor provided herein may be multispecific. The extracellular antigen binding domain of the chimeric cytokine receptor provided herein may be monovalent. The extracellular antigen binding domain of the chimeric cytokine receptor provided herein may be multivalent. The extracellular antigen binding domain may comprise two or more antigen binding domains which are fused to each other directly via peptide bonds, or via peptide linkers.

### Functional Exogenous Receptors (e.g., CAR and TCR)

In one aspect, the present disclosure provides cells (e.g., immune cells) that have the chimeric cytokine receptors described herein and a functional exogenous receptor. The functional exogenous receptor may comprise an extracellular antigen binding domain, and optionally an intracellular signaling domain. Exemplary functional exogenous receptors include, but are not limited to, CAR, engineered TCR, and TAC receptors. The functional exogenous receptor may comprise an extracellular domain comprising an antigen binding domain that specifically binds to an antigen (e.g., a tumor antigen), a transmembrane domain, and an intracellular signaling domain. The intracellular signaling domain may comprise a primary intracellular signaling domain and/or a co-stimulatory signaling domain. The intracellular signaling domain may comprise an intracellular signaling domain of a TCR co-receptor. The functional exogenous receptor may be encoded by a heterologous polynucleotide operably linked to a promoter (such as a constitutive promoter or an inducible promoter).

The functional exogenous receptor may comprise one or more specific binding domains that target at least one tumor antigen, and one or more intracellular effector domains, such as one or more primary intracellular signaling domains and/or co-stimulatory signaling domains.

The functional exogenous receptor may be a chimeric antigen receptor (CAR). CARS can also be constructed with a specificity for any cell surface marker by utilizing antigen binding fragments or antibody variable domains of, for example, antibody molecules.

CARs of the present disclosure comprise an extracellular domain comprising at least one antigen binding domain that specifically binds at least one tumor antigen (e.g., a extracellular antigen binding domain), a transmembrane domain, and an intracellular signaling domain. The intracellular signaling domain may generate a signal that promotes an immune effector function of the CAR-containing cell, e.g., a CAR-T cell. “Immune effector function or immune effector response” refers to function or response, e.g., of an immune effector cell, that enhances or promotes an immune attack of a target cell. For example, an immune effector function or response can refer to a property of a T or NK cell that promotes killing or the inhibition of growth or proliferation, of a target cell. Examples of immune effector function, e.g., in a CAR-T cell, include cytolytic activity and helper activity (such as the secretion of cytokines). The intracellular signaling domain may generate a signal that promotes proliferation and/or survival of the CAR containing cell. The CAR may comprise one or more intracellular signaling domains selected from the signaling domains of CD28, CD137, CD3, CD27, CD40, ICOS, GITR, and OX40. The signaling domain of a naturally occurring molecule can comprise the entire intracellular or cytoplasmic portion, or the entire native intracellular signaling domain, of the molecule, or a fragment or derivative thereof.

The intracellular signaling domain of a CAR may comprise a primary intracellular signaling domain. “Primary intracellular signaling domain” refers to cytoplasmic signaling sequence that acts in a stimulatory manner to induce immune effector functions. The primary intracellular signaling domain may contain a signaling motif known as Immunoreceptor Tyrosine-based Activation Motif, or ITAM. The primary intracellular signaling domain may comprise a functional signaling domain of a protein selected from the group consisting of CD3 zeta, CD3 gamma, CD3 delta, CD3 epsilon, common FcR gamma (FCER1G), FcR beta (Fc Epsilon Rib), CD79a, CD79b, Fcgamma R IIa, DAP10, and DAP12. The primary intracellular signaling domain may comprise a nonfunctional or attenuated signaling domain of a protein selected from the group consisting of CD3 zeta, CD3 gamma, CD3 delta, CD3 epsilon, common FcR gamma (FCER1G), FcR beta (Fc Epsilon Rib), CD79a, CD79b, Fcgamma R IIa, DAP10, and DAP12. The nonfunctional or attenuated signaling domain can be a mutant signaling domain having a point mutation, insertion or deletion that attenuates or abolishes one or more immune effector functions, such as cytolytic activity or helper activity. The CAR may comprise a nonfunctional or attenuated CD3 zeta (i.e. CD3ζ or CD3z) signaling domain. The intracellular signaling domain may not comprise a primary intracellular signaling domain.

The intracellular signaling domain of a CAR may comprise one or more (such as any of 1, 2, 3, or more) co-stimulatory signaling domains. “Co-stimulatory signaling domain” can be the intracellular portion of a co-stimulatory molecule. The term “co-stimulatory molecule” refers to a cognate binding partner on an immune cell (such as T cell) that specifically binds with a co-stimulatory ligand, thereby mediating a co-stimulatory response by the immune cell, such as, but not limited to, proliferation and survival. Co-stimulatory molecules are cell surface molecules other than antigen receptors or their ligands that contribute to an efficient immune response. A co-stimulatory molecule can be represented in the following protein families: TNF receptor proteins, Immunoglobulin-like proteins, cytokine receptors, integrins, signaling lymphocytic activation molecules (SLAM proteins), and activating NK cell receptors. Co-stimulatory molecules include, but are not limited to an MHC class I molecule, BTLA and a Toll ligand receptor, as well as OX40, CD27, CD28, CDS, ICAM-1, LFA-1 (CD11a/CD18), ICOS (CD278), and 4-1BB (CD137). Further examples of such co-stimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CDIOO (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

The CAR may comprise a single co-stimulatory signaling domain. The CAR may comprise two or more co-stimulatory signaling domains. The intracellular signaling domain may comprise a functional primary intracellular signaling domain and one or more co-stimulatory signaling domains. The CAR may not comprise a functional primary intracellular signaling domain (such as CD32). The CAR may comprise an intracellular signaling domain consisting of or consisting essentially of one or more co-stimulatory signaling domains. The CAR may comprise an intracellular signaling domain consisting of or consisting essentially of a nonfunctional or attenuated primary intracellular signaling domain (such as a mutant CD32) and one or more co-stimulatory signaling domains. Upon binding of the antigen binding domain to tumor antigen, the co-stimulatory signaling domains of the CAR can transduce signals for enhanced proliferation, survival and differentiation of the modified immune cells having the CAR (such as T cells), and inhibit activation induced cell death. The one or more co-stimulatory signaling domains may be derived from one or more molecules selected from the group consisting of CD27, CD28, 4-1BB (i.e., CD137), OX40, CD30, CD40, CD3, lymphocyte function-associated antigen-1 (LFA-1), CD2, CD7, LIGHT, NKG2C, B7-H3 and ligands that specially bind to CD83.

The intracellular signaling domain of a CAR may comprise a co-stimulatory signaling domain derived from CD28. The intracellular signaling domain may comprise a cytoplasmic signaling domain of CD32 and a co-stimulatory signaling domain of CD28. The intracellular signaling domain in the chimeric receptor of the present application may comprise a co-stimulatory signaling domain derived from 4-1BB (i.e., CD137). The intracellular signaling domain may comprise a cytoplasmic signaling domain of CD3ζ and a co-stimulatory signaling domain of 4-1BB.

The intracellular signaling domain of the CAR may comprise a co-stimulatory signaling domain of CD28 and a co-stimulatory signaling domain of 4-1BB. The intracellular signaling domain may comprise a cytoplasmic signaling domain of CD3ζ, a co-stimulatory signaling domain of CD28, and a co-stimulatory signaling domain of 4-1BB. The intracellular signaling domain may comprise a polypeptide comprising from the N-terminus to the C-terminus: a co-stimulatory signaling domain of CD28, a co-stimulatory signaling domain of 4-1BB, and a cytoplasmic signaling domain of CD3ζ.

The extracellular antigen binding domain of a CAR (e.g., the extracellular antigen binding domain) may be an antibody or an antibody fragment, such as an scFv, a Fv, a Fab, a (Fab′)₂, a single domain antibody (sdAb), or a V_{H}H domain. The antigen binding domain of a CAR may comprise a ligand or an extracellular portion of a receptor that specifically binds to a tumor antigen. The CAR may be a monospecific, bispecific or multispecific CAR. The antigen binding domain of a CAR may specifically bind a single tumor antigen. The antigen binding domain of a CAR may bind two or more tumor antigens.

The transmembrane domain of a CAR (e.g., the transmembrane domain) may comprise a transmembrane domain chosen from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL-2R beta, IL-2R gamma, IL-7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C. The transmembrane domain of the CAR may be a CD4, CD3, CD8α, or CD28 transmembrane domain. The transmembrane domain of the CAR may comprise a transmembrane domain of CD8α.

The extracellular domain may be connected to the transmembrane domain by a hinge region. The hinge region may comprise a hinge region of CD8α. The CD8α hinge region may comprise an amino acid sequence set forth in SEQ ID NO: 86 or an amino acid sequence that is at least 80%, 85%, 90%, 95% or 99% identical to the amino acid sequence set forth in SEQ ID NO: 86.

The CAR may comprise a signal peptide (SP), such as a CD8α signal peptide (SEQ ID NO: 1).

The CAR may bind to a tumor-associated antigen. The tumor-associated antigen may be selected from the group consisting of CD19, AFP, BCMA, NY-ESO-1, VEGFR2, MAGE-A3, VEGFR2, CD20, CD22, CD30, CD33, GUCY2C, MUC17, FGFR2, CLL1, CD38, CEA, CS1, CD138, CD123/IL3Rα, c-Met, gp100, MUC1, IGF-I receptor, EpCAM, CEA, EGFR (such as EGFRVIII), GD2, HER2, IGFIR, mesothelin, PSMA, ROR1, WT1, Glypican 3 (GPC3), Guanylate cyclase 2C (GCC), DLL3, Claudin18.2, Claudin6, Glycolipid F77, PD-L1, PD-L2, and other tumor antigens with clinical significance, and combinations thereof. The tumor antigen may be derived from an intracellular protein of tumor cells. The tumor antigen may be expressed on the surface of tumor cells.

Many CARs targeting different tumor antigens have been widely disclosed in the field, such as CD19 CARs or BCMA CARs. The extracellular antigen-binding domain of CD19 CARs can be or include the CD19 binding fragment (e.g., FMC63, SJ25C1, or those disclosed in different patents such as WO 2022/012683, etc). BCMA CARs also have been well described, related patents include but not limited to WO 2016/014789, WO 2016/014565, WO 2013/154760, and WO 2018/028647, etc. Glypican 3 (GPC3), also called OCI-5, DGSX, GTR2-2, MXR7, SDYS, SGB, SGBS, and SGBS1, is a glycosylphosphatidylinositol-anchored cell surface protein consisting of a core protein and two heparan sulfate (HS) chains. GPC3 belongs to the glypican-related integral membrane proteoglycan family, which includes six members (GPC1-GPC6). According to the homogeneity and heterogeneity of gene sequences, these six members are divided into two subfamilies; one group is GPC3 and GPC5 which show 43% sequence homology, while the other group contains GPC1, GPC2, GPC4, and GPC6. GPC3 is encoded at chromosome Xp26 adjacent to GPC4, and spans more than 500 kilobases. Four isoforms have been reported, of which isoform 2 (GenBank Accession No.: NP_004475), which encodes a 70-kDa precursor core protein with 580 amino acids, is the most commonly expressed.

GPC3 is an oncofetal protein expressed in over 70% of HCC and other solid tumors including hepatoblastoma and lung squamous cell carcinoma. Its expression is not detected in nonmalignant adult tissues including normal liver. Mechanistically, GPC3 can promote tumor growth by modulating the Wnt/Frizzled signaling complex on HCC cells. Considering the highly specific expression of GPC3 in hepatocellular carcinoma, melanoma and other tumors, it is considered as a candidate target antigen for CAR-T cells tumor immunotherapy.

The CAR may comprise a single chain variable fragment (scFv) comprising the following sequences or sequences that are at least 80%, 85%, 90%, 95%, 96%, 97%, 98%, 99% identical to: a heavy chain complementarity determining region 1 (CDR1), CDR2, CDR3, a light chain complementarity determining region 1 (CDR1), CDR2, and CDR3, having the amino acid sequences of: (1) SEQ ID NOs: 38, 39, 40, 41, 42, 43, respectively; (2) SEQ ID NOs: 44, 45, 46, 47, 48, 49, respectively; (3) SEQ ID NOs: 50, 51, 52, 53, 54, 55, respectively; or (4) SEQ ID NOs: 97, 98, 99, 100, 101, 102, respectively.

The CAR may comprise a single chain variable fragment (scFv) comprising: a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 38, 39, 40, respectively, and the selected VL CDRs 1, 2, 3 amino acid sequences are set forth in SEQ ID NOs: 41, 42, 43, respectively;
(2) the selected VH CDRs 1, 2, 3 amino acid sequences are set forth in SEQ ID NOs: 44, 45, 46, respectively, and the selected VL CDRs 1, 2, 3 amino acid sequences are set forth in SEQ ID NOs: 47, 48, 49, respectively;
(3) the selected VH CDRs 1, 2, 3 amino acid sequences are set forth in SEQ ID NOs: 50, 51, 52, respectively, and the selected VL CDRs 1, 2, 3 amino acid sequences are set forth in SEQ ID NOs: 53, 54, 55, respectively; and
(4) the selected VH CDRs 1, 2, 3 amino acid sequences are set forth in SEQ ID NOs: 97, 98, 99, respectively, and the selected VL CDRs 1, 2, 3 amino acid sequences are set forth in SEQ ID NOs: 100, 101, 102, respectively.

The CAR may comprise a heavy chain variable region (VH) comprising CDR1, CDR2, and CDR3 that are identical to CDR1, CDR2, and CDR3 of a VH sequence, and a light chain variable region (VL) comprising CDR1, CDR2, and CDR3 that are identical to CDR1, CDR2, and CDR3 of a VL sequence, wherein (1) the VH sequence is SEQ ID NO: 72 and the VL sequence is SEQ ID NO: 73; (2) the VH sequence is SEQ ID NO: 74, and the VL sequence is SEQ ID NO: 75; (3) the VH sequence is SEQ ID NO: 76, and the VL sequence is SEQ ID NO: 77; or (4) the VH sequence is SEQ ID NO: 94, and the VL sequence is SEQ ID NO: 95.

The CAR may comprise a heavy chain variable region (VH) comprising an amino acid sequence that is at least 80%, 85%, 90%, 95%, 96%, 97%, 98%, 99% or 100% identical to a selected VH sequence, and a light chain variable region (VL) comprising an amino acid sequence that is at least 80%, 85%, 90%, 95%, 96%, 97%, 98%, 99% or 100% identical to a selected VL sequence, wherein the selected VH sequence and the selected VL sequence are one of the following:
(1) the selected VH sequence is SEQ ID NO: 72, and the selected VL sequence is SEQ ID NO: 73;
(2) the selected VH sequence is SEQ ID NO: 74, and the selected VL sequence is SEQ ID NO: 75;
(3) the selected VH sequence is SEQ ID NO: 76, and the selected VL sequence is SEQ ID NO: 77; and
(4) the selected VH sequence is SEQ ID NO: 94, and the selected VL sequence is SEQ ID NO: 95.

The CAR may comprise a V_{H}H that having a sequence that is at least 80%, 85%, 90%, 95%, 96%, 97%, 98%, 99% or 100% identical to: a CDR1 comprising the amino acid sequence of SEQ ID NO: 56, a CDR2 comprising the amino acid sequence of SEQ ID NO: 57, and a CDR3 comprising the amino acid sequence of SEQ ID NO: 58; a CDR1 comprising the amino acid sequence of SEQ ID NO: 59, a CDR2 comprising the amino acid sequence of SEQ ID NO: 60, and a CDR3 comprising the amino acid sequence of SEQ ID NO: 61; a CDR1 comprising the amino acid sequence of SEQ ID NO: 62, a CDR2 comprising the amino acid sequence of SEQ ID NO: 63, and a CDR3 comprising the amino acid sequence of SEQ ID NO: 64; or a CDR1 comprising the amino acid sequence of SEQ ID NO: 65, a CDR2 comprising the amino acid sequence of SEQ ID NO: 66, and a CDR3 comprising the amino acid sequence of SEQ ID NO: 67.

The CAR may comprise a V_{H}H comprising the amino acid sequences of any one of SEQ ID NOs: 68-71 or an amino acid sequence having at least 80%, 85%, 90%, 95%, 96%, 97%, 98%, 99% or 100% identity to the amino acid sequence of any one of SEQ ID NOs: 68-71.

The CAR may comprise an amino acid sequence set forth in any one of SEQ ID NOs: 4, 78-83 and 91, or an amino acid sequence having at least 90%, 95%, or 99% identity to the amino acid sequence set forth in any one of SEQ ID NOs: 4, 78-83 and 91.

The intracellular signaling domain of the CAR may comprise any suitable elements known in the art. The intracellular signaling domain of the CAR may comprise a primary intracellular signaling domain of a cell. The primary intracellular signaling domain may be derived from CD3ζ.

The intracellular signaling domain may comprise a co-stimulatory signaling domain. The co-stimulatory signaling domain can be derived from the intracellular domains of a wild-type co-stimulatory protein or a functional variant thereof. The co-stimulatory signaling domain can have one or more mutations, including e.g., insertions, deletions, and/or substitutions.

The co-stimulatory signaling domain may be derived from a co-stimulatory molecule selected from the group consisting of CD27, CD28, CD137, OX40, CD30, CD40, CD3, LFA-1, ICOS, CD2, CD7, LIGHT, NKG2C, B7-H3, ligands of CD83 and combinations thereof.

Also described herein is an antibody or antigen-binding fragment thereof, comprising:
a single chain variable fragment (scFv) comprising a heavy chain complementarity determining region 1 (CDR1), CDR2, CDR3, a light chain complementarity determining region 1 (CDR1), CDR2, and CDR3, having the amino acid sequences of:
(1) SEQ ID NOs: 38, 39, 40, 41, 42, 43, respectively;
(2) SEQ ID NOs: 44, 45, 46, 47, 48, 49, respectively;
(3) SEQ ID NOs: 50, 51, 52, 53, 54, 55, respectively; or
(4) SEQ ID NOs: 97, 98, 99, 100, 101, 102, respectively.

Also described herein is an antibody or antigen-binding fragment thereof, comprising: a heavy chain variable region (VH) comprising CDR1, CDR2, and CDR3 and a light chain variable region (VL) comprising CDR1, CDR2, and CDR3, having the amino acid sequences of:
(1) the VH sequence is SEQ ID NO: 72 and the VL sequence is SEQ ID NO: 73;
(2) the VH sequence is SEQ ID NO: 74, and the VL sequence is SEQ ID NO: 75;
(3) the VH sequence is SEQ ID NO: 76, and the VL sequence is SEQ ID NO: 77; or
(3) the VH sequence is SEQ ID NO: 94, and the VL sequence is SEQ ID NO: 95.

Also described herein is a V_{H}H antibody moiety having the amino acid sequences of:
(1) a CDR1 comprising the amino acid sequence of SEQ ID NO: 56, a CDR2 comprising the amino acid sequence of SEQ ID NO: 57, and a CDR3 comprising the amino acid sequence of SEQ ID NO: 58;
(2) a CDR1 comprising the amino acid sequence of SEQ ID NO: 59, a CDR2 comprising the amino acid sequence of SEQ ID NO: 60, and a CDR3 comprising the amino acid sequence of SEQ ID NO: 61;
(3) a CDR1 comprising the amino acid sequence of SEQ ID NO: 62, a CDR2 comprising the amino acid sequence of SEQ ID NO: 63, and a CDR3 comprising the amino acid sequence of SEQ ID NO: 64; or
(4) a CDR1 comprising the amino acid sequence of SEQ ID NO: 65, a CDR2 comprising the amino acid sequence of SEQ ID NO: 66, and a CDR3 comprising the amino acid sequence of SEQ ID NO: 67.

The V_{H}H antibody moiety may comprise the amino acid sequences of any one of SEQ ID NOs: 68-71 or an amino acid sequence having at least 90%, 95%, or 99% identity to the amino acid sequence of any one of SEQ ID NOs: 68-71.

The functional exogenous receptor may be a T-cell receptor (e.g., an engineered TCR). The engineered TCR may be specific for a tumor antigen. Many TCRs specific for tumor antigens (including tumor-associated antigens) have been described, including, for example, NY-ESO-1 cancer-testis antigen, the p53 tumor suppressor antigens, TCRs for tumor antigens in melanoma (e.g., MARTI, gp 100), leukemia (e.g., WT1, minor histocompatibility antigens), and breast cancer (e.g., HER2, NY-BR1). Any of the TCRs known in the art can be used. The TCR may have an enhanced affinity to the tumor antigen. Exemplary TCRs and methods for introducing the TCRs to immune cells have been described, for example, in U.S. Pat. No. 5,830,755, and Kessels et al. Immunotherapy through TCR gene transfer. Nat. Immunol. 2, 957-961 (2001), which are incorporated herein by reference in the entirety.

The TCR receptor complex is an octomeric complex formed by variable TCR receptor α and β chains (or γ and δ chains on case of γδ T cells) with three dimeric signaling modules CD3δ/ε, CD3γ/ε and CD247 (T-cell surface glycoprotein CD3 zeta chain) ζ/ζ or ζ/n. Ionizable residues in the transmembrane domain of each subunit form a polar network of interactions that hold the complex together. TCR complex has the function of activating signaling cascades in T cells.

The functional exogenous receptor may be an engineered TCR comprising one or more T-cell receptor (TCR) fusion proteins (TFPs). Exemplary TFPs have been described, for example, in US20170166622A1, which is incorporated herein by reference in its entirety. The TFP may comprise an extracellular domain of a TCR subunit that comprises an extracellular domain or portion thereof of a protein selected from the group consisting of a TCR alpha chain, a TCR beta chain, a CD3 epsilon TCR subunit, a CD3 gamma TCR subunit, a CD3 delta TCR subunit, functional fragments thereof, and amino acid sequences thereof having at least one but not more than 20 modifications. The TFP may comprise a transmembrane domain that comprises a transmembrane domain of a protein selected from the group consisting of a TCR alpha chain, a TCR beta chain, a CD3 epsilon TCR subunit, a CD3 gamma TCR subunit, a CD3 delta TCR subunit, functional fragments thereof, and variants thereof (e.g., amino acid sequences having at least one but not more than 20 modifications). The TFP may comprise a transmembrane domain that comprises a transmembrane domain of a protein selected from the group consisting of a TCR alpha chain, a TCR beta chain, a TCR zeta chain, a CD3 epsilon TCR subunit, a CD3 gamma TCR subunit, a CD3 delta TCR subunit, CD45, CD4, CD5, CD8, CD9, CD16, CD22, CD33, CD28, CD37, CD64, CD80, CD86, CD134, CD137, CD154, functional fragments thereof, and variants thereof (e.g., amino acid sequences thereof having at least one but not more than 20 modifications).

The TFP comprising a TCR subunit may comprise at least a portion of a TCR extracellular domain, and a TCR intracellular domain comprising a stimulatory domain from an intracellular signaling domain of CD3 epsilon; and an antigen binding domain, wherein the TCR subunit and the antigen binding domain are operatively linked, and wherein the TFP incorporates into a TCR when expressed in a T cell.

The functional exogenous receptor may be a T-cell antigen coupler (TAC) receptor. Exemplary TAC receptors have been described, for example, in US20160368964A1, which is incorporated herein by reference in its entirety. The TAC may comprise an antigen binding domain, a TCR-binding domain that specifically binds a protein associated with the TCR complex, and a T-cell receptor signaling domain. The antigen binding domain may be an antibody fragment, such as scFv or V_{H}H, which specifically binds to a tumor antigen. The antigen binding domain may be a designed Ankyrin repeat (DARPin) polypeptide. The tumor antigen may be e.g., CD19, BCMA, NY-ESO-1, VEGFR2, MAGE-A3, VEGFR2, MAGE-A3, CD20, CD22, CD30, CD33, CD38, CEA, CS1, CD138, CD123/IL3Rα, c-Met, gp100, MUC1, IGF-I receptor, EpCAM, CEA, EGFR (such as EGFRvIII), GD2, HER2, IGF1R, mesothelin, PSMA, ROR1, WT1, Glypican 3 (GPC3), Guanylate cyclase 2C (GCC), DLL3, Claudin18.2, Claudin6, Glycolipid F77, PD-L1, PD-L2, or other tumor antigens with clinical significance, or combinations thereof. The tumor antigen may be derived from an intracellular protein of tumor cells. The tumor antigen may be expressed on the surface of tumor cells. The protein associated with the TCR complex may be CD3, such as CD3e. The TCR-binding domain may be a single chain antibody, such as scFv, or a V_{H}H. The TAC receptor may comprise a cytosolic domain and a transmembrane domain. The T-cell receptor signaling domain may comprise a cytosolic domain derived from a TCR co-receptor. Exemplary TCR co-receptors include, but are not limited to, CD4, CD8, CD28, CD45, CD4, CD5, CD9, CD16, CD22, CD33, CD37, CD64, CD80, CD86, CD134, CD137 and CD 154. The TAC receptor may comprise a transmembrane domain and a cytosolic domain derived from CD4. The TAC receptor may comprise a transmembrane domain and a cytosolic domain derived from CD8 (such as CD8α).

T cell co-receptors are expressed as membrane protein on T cells. They can provide stabilization of the TCR: peptide: MHC complex and facilitate signal transduction. The two subtypes of T cell co-receptor, CD4 and CD8, display strong specificity for particular MHC classes. The CD4 co-receptor can only stabilize TCR: MHC II complexes while the CD8 co-receptor can only stabilize the TCR: MHC I complex. The differential expression of CD4 and CD8 on different T cell types results in distinct T cell functional subpopulations. CD8+ T cells are cytotoxic T cells.

### Genetically Engineered Cells

The present disclosure provides cells (e.g., genetically engineered immune cells, T cells, NK cells, tumor-infiltrating lymphocytes) that express the chimeric cytokine receptor described herein. These engineered cells can be used to treat various disorders or disease as described herein (e.g., a cancer).

The cell may further comprise a functional exogenous receptor. The functional exogenous receptor may comprise: (a) an extracellular antigen binding domain, (b) a transmembrane domain, and (c) an intracellular signaling domain.

The chimeric cytokine receptor and the functional exogenous receptor may be linked to each other via a peptide linker. The peptide linker can be any suitable linker known in the art. The peptide linker may be a self-cleaving peptide linker. The self-cleaving peptide linker may be a 2A self-cleaving peptide. The 2A self-cleaving peptide may be selected from a group consisting of F2A, E2A, P2A, T2A, and variants thereof.

The cell may comprise the amino acid sequence set forth in any one of SEQ ID NOs: 22-37, 92 and 93, or an amino acid sequence that is at least 80%, 85%, 90%, 95% or 99% identical to the amino acid sequence set forth in any one of SEQ ID NOs: 22-37, 92 and 93.

The cell may be an immune cell. The immune cell may be selected from a group consisting of T cell, NK cell, NKT, peripheral blood mononuclear cell (PBMC), hematopoietic stem cell, pluripotent stem cell, an embryonic stem cell, a macrophage, a monocyte, a neutrophil, an eosinophil and a combination thereof.

The T cell may be a αβT cell or γδT cell. The T cell, upon activation, may exhibit increased expression of pSTAT3 and pSTAT5.

The cell may comprise a CAR backbone sequence encoding a CAR backbone polypeptide comprising from the N-terminus to the C-terminus: a signal peptide (e.g., SEQ ID NO: 1 or a sequence that is at least 80%, 85%, 90%, or 95% identical to SEQ ID NO: 1), a GPC3-CAR (e.g., SEQ ID NO: 4 or a sequence that is at least 80%, 85%, 90%, or 95% identical to SEQ ID NO: 4), via a P2A cleavage site (e.g., SEQ ID NO: 5 or a sequence that is at least 80%, 85%, 90%, or 95% identical to SEQ ID NO: 5), and is further connected to a NKG2D chimeric cytokine receptor molecule comprising from the N-terminus to the C-terminus: a signal peptide (e.g., SEQ ID NO: 1 or a sequence that is at least 80%, 85%, 90%, or 95% identical to SEQ ID NO: 1), a NKG2D ECD (e.g., SEQ ID NO: 6 or a sequence that is at least 80%, 85%, 90%, or 95% identical to SEQ ID NO: 6), a TPOR transmembrane (TM) (e.g., SEQ ID NO: 9 or a sequence that is at least 80%, 85%, 90%, or 95% identical to SEQ ID NO: 9) and TPOR BOX1-BOX2 (e.g., SEQ ID NO: 7 or a sequence that is at least 80%, 85%, 90%, or 95% identical to SEQ ID NO: 7), a IL-15Rβ ICD (e.g., SEQ ID NO: 10 or a sequence that is at least 80%, 85%, 90%, or 95% identical to SEQ ID NO: 10) and a IL-21R ICD (e.g., SEQ ID NO: 20 or a sequence that is at least 80%, 85%, 90%, or 95% identical to SEQ ID NO: 20). The cell may comprise an amino acid sequence set forth in SEQ ID NO: 22, or an amino acid sequence that is at least 80%, 85%, 90%, 95% or 99% identical to the amino acid sequence set forth in SEQ ID NO: 22.

The cell may comprise a CAR backbone sequence encoding a CAR backbone polypeptide comprising from the N-terminus to the C-terminus: a signal peptide (e.g., SEQ ID NO: 1 or a sequence that is at least 80%, 85%, 90%, or 95% identical to SEQ ID NO: 1), a GPC3-CAR (e.g., SEQ ID NO: 4 or a sequence that is at least 80%, 85%, 90%, or 95% identical to SEQ ID NO: 4), via a P2A cleavage site (e.g., SEQ ID NO: 5 or a sequence that is at least 80%, 85%, 90%, or 95% identical to SEQ ID NO: 5), and is further connected to a NKG2D chimeric cytokine receptor molecule comprising from the N-terminus to the C-terminus: a signal peptide (e.g., SEQ ID NO: 1 or a sequence that is at least 80%, 85%, 90%, or 95% identical to SEQ ID NO: 1), a NKG2D ECD (e.g., SEQ ID NO: 6 or a sequence that is at least 80%, 85%, 90%, or 95% identical to SEQ ID NO: 6), a CD8 hinge (e.g., SEQ ID NO: 86 or a sequence that is at least 80%, 85%, 90%, or 95% identical to SEQ ID NO: 86), a CD8TM (e.g., SEQ ID NO: 2 or a sequence that is at least 80%, 85%, 90%, or 95% identical to SEQ ID NO: 2), a TPOR BOX1-BOX2 (e.g., SEQ ID NO: 7 or a sequence that is at least 80%, 85%, 90%, or 95% identical to SEQ ID NO: 7), a IL-15Rβ ICD (e.g., SEQ ID NO: 10 or a sequence that is at least 80%, 85%, 90%, or 95% identical to SEQ ID NO: 10) and a IL-21R ICD (e.g., SEQ ID NO: 20 or a sequence that is at least 80%, 85%, 90%, or 95% identical to SEQ ID NO: 20). The cell may comprise an amino acid sequence set forth in SEQ ID NO: 23, or an amino acid sequence that is at least 80%, 85%, 90%, 95% or 99% identical to the amino acid sequence set forth in SEQ ID NO: 23.

The cell may comprise a CAR backbone sequence encoding a CAR backbone polypeptide comprising from the N-terminus to the C-terminus: a signal peptide (e.g., SEQ ID NO: 1 or a sequence that is at least 80%, 85%, 90%, or 95% identical to SEQ ID NO: 1), a GPC3-CAR (e.g., SEQ ID NO: 4 or a sequence that is at least 80%, 85%, 90%, or 95% identical to SEQ ID NO: 4), via a P2A cleavage site (e.g., SEQ ID NO: 5 or a sequence that is at least 80%, 85%, 90%, or 95% identical to SEQ ID NO: 5), and is further connected to a NKG2D chimeric cytokine receptor molecule comprising from the N-terminus to the C-terminus: a signal peptide (e.g., SEQ ID NO: 1 or a sequence that is at least 80%, 85%, 90%, or 95% identical to SEQ ID NO: 1), a NKG2D ECD (e.g., SEQ ID NO: 6 or a sequence that is at least 80%, 85%, 90%, or 95% identical to SEQ ID NO: 6), a CD28 TM (e.g., SEQ ID NO: 3 or a sequence that is at least 80%, 85%, 90%, or 95% identical to SEQ ID NO: 3), a TPOR BOX1-BOX2 (e.g., SEQ ID NO: 7 or a sequence that is at least 80%, 85%, 90%, or 95% identical to SEQ ID NO: 7), a IL-15Rβ ICD (e.g., SEQ ID NO: 10 or a sequence that is at least 80%, 85%, 90%, or 95% identical to SEQ ID NO: 10) and a IL-21R ICD (e.g., SEQ ID NO: 20 or a sequence that is at least 80%, 85%, 90%, or 95% identical to SEQ ID NO: 20). The cell may comprise an amino acid sequence set forth in SEQ ID NO: 24, or an amino acid sequence that is at least 80%, 85%, 90%, 95% or 99% identical to the amino acid sequence set forth in SEQ ID NO: 24.

The cell may comprise a CAR backbone sequence encoding a CAR backbone polypeptide comprising from the N-terminus to the C-terminus: a signal peptide (e.g., SEQ ID NO: 1 or a sequence that is at least 80%, 85%, 90%, or 95% identical to SEQ ID NO: 1), a GPC3-CAR (e.g., SEQ ID NO: 4 or a sequence that is at least 80%, 85%, 90%, or 95% identical to SEQ ID NO: 4), via a P2A cleavage site (e.g., SEQ ID NO: 5 or a sequence that is at least 80%, 85%, 90%, or 95% identical to SEQ ID NO: 5), and is further connected to a NKG2D chimeric cytokine receptor molecule comprising from the N-terminus to the C-terminus: a signal peptide (e.g., SEQ ID NO: 1 or a sequence that is at least 80%, 85%, 90%, or 95% identical to SEQ ID NO: 1), a NKG2D ECD (e.g., SEQ ID NO: 6 or a sequence that is at least 80%, 85%, 90%, or 95% identical to SEQ ID NO: 6), a TPOR TM (e.g., SEQ ID NO: 9 or a sequence that is at least 80%, 85%, 90%, or 95% identical to SEQ ID NO: 9) and TPOR-BOX1 (e.g., SEQ ID NO: 8 or a sequence that is at least 80%, 85%, 90%, or 95% identical to SEQ ID NO: 8), a IL-15Rβ ICD (e.g., SEQ ID NO: 10 or a sequence that is at least 80%, 85%, 90%, or 95% identical to SEQ ID NO: 10) and a IL-21R ICD (e.g., SEQ ID NO: 20 or a sequence that is at least 80%, 85%, 90%, or 95% identical to SEQ ID NO: 20). The cell may comprise an amino acid sequence set forth in SEQ ID NO: 25, or an amino acid sequence that is at least 80%, 85%, 90%, 95% or 99% identical to the amino acid sequence set forth in SEQ ID NO: 25.

The cell may comprise a CAR backbone sequence encoding a CAR backbone polypeptide comprising from the N-terminus to the C-terminus: a signal peptide (e.g., SEQ ID NO: 1 or a sequence that is at least 80%, 85%, 90%, or 95% identical to SEQ ID NO: 1), a GPC3-CAR (e.g., SEQ ID NO: 4 or a sequence that is at least 80%, 85%, 90%, or 95% identical to SEQ ID NO: 4), via a P2A cleavage site (e.g., SEQ ID NO: 5 or a sequence that is at least 80%, 85%, 90%, or 95% identical to SEQ ID NO: 5), and is further connected to a NKG2D chimeric cytokine receptor molecule comprising from the N-terminus to the C-terminus: a signal peptide (e.g., SEQ ID NO: 1 or a sequence that is at least 80%, 85%, 90%, or 95% identical to SEQ ID NO: 1), a NKG2D ECD (e.g., SEQ ID NO: 6 or a sequence that is at least 80%, 85%, 90%, or 95% identical to SEQ ID NO: 6), a TPOR TM (e.g., SEQ ID NO: 9 or a sequence that is at least 80%, 85%, 90%, or 95% identical to SEQ ID NO: 9) and IL-15Rβ BOX1-BOX2 (e.g., SEQ ID NO: 19 or a sequence that is at least 80%, 85%, 90%, or 95% identical to SEQ ID NO: 19), a IL-15Rβ ICD (e.g., SEQ ID NO: 10 or a sequence that is at least 80%, 85%, 90%, or 95% identical to SEQ ID NO: 10) and a IL-21R ICD (e.g., SEQ ID NO: 20 or a sequence that is at least 80%, 85%, 90%, or 95% identical to SEQ ID NO: 20). The cell may comprise an amino acid sequence set forth in SEQ ID NO: 26, or an amino acid sequence that is at least 80%, 85%, 90%, 95% or 99% identical to the amino acid sequence set forth in SEQ ID NO: 26.

The cell may comprise a CAR backbone sequence encoding a CAR backbone polypeptide comprising from the N-terminus to the C-terminus: a signal peptide (e.g., SEQ ID NO: 1 or a sequence that is at least 80%, 85%, 90%, or 95% identical to SEQ ID NO: 1), a GPC3-CAR (e.g., SEQ ID NO: 4 or a sequence that is at least 80%, 85%, 90%, or 95% identical to SEQ ID NO: 4), via a P2A cleavage site (e.g., SEQ ID NO: 5 or a sequence that is at least 80%, 85%, 90%, or 95% identical to SEQ ID NO: 5), and is further connected to a NKG2D chimeric cytokine receptor molecule comprising from the N-terminus to the C-terminus: a signal peptide (e.g., SEQ ID NO: 1 or a sequence that is at least 80%, 85%, 90%, or 95% identical to SEQ ID NO: 1), a NKG2D ECD (e.g., SEQ ID NO: 6 or a sequence that is at least 80%, 85%, 90%, or 95% identical to SEQ ID NO: 6), a CD28 TM (e.g., SEQ ID NO: 3 or a sequence that is at least 80%, 85%, 90%, or 95% identical to SEQ ID NO: 3), a TPOR-BOX1 (e.g., SEQ ID NO: 8 or a sequence that is at least 80%, 85%, 90%, or 95% identical to SEQ ID NO: 8), a IL-15Rβ ICD (e.g., SEQ ID NO: 10 or a sequence that is at least 80%, 85%, 90%, or 95% identical to SEQ ID NO: 10) and a IL-21R ICD (e.g., SEQ ID NO: 20 or a sequence that is at least 80%, 85%, 90%, or 95% identical to SEQ ID NO: 20). The cell may comprise an amino acid sequence set forth in SEQ ID NO: 27, or an amino acid sequence that is at least 80%, 85%, 90%, 95% or 99% identical to the amino acid sequence set forth in SEQ ID NO: 27.

The cell may comprise a CAR backbone sequence encoding a CAR backbone polypeptide comprising from the N-terminus to the C-terminus: a signal peptide (e.g., SEQ ID NO: 1 or a sequence that is at least 80%, 85%, 90%, or 95% identical to SEQ ID NO: 1), a GPC3-CAR (e.g., SEQ ID NO: 4 or a sequence that is at least 80%, 85%, 90%, or 95% identical to SEQ ID NO: 4), via a P2A cleavage site (e.g., SEQ ID NO: 5 or a sequence that is at least 80%, 85%, 90%, or 95% identical to SEQ ID NO: 5), and is further connected to a NKG2D chimeric cytokine receptor molecule comprising from the N-terminus to the C-terminus: a signal peptide (e.g., SEQ ID NO: 1 or a sequence that is at least 80%, 85%, 90%, or 95% identical to SEQ ID NO: 1), a NKG2D ECD (e.g., SEQ ID NO: 6 or a sequence that is at least 80%, 85%, 90%, or 95% identical to SEQ ID NO: 6), a TPOR TM (e.g., SEQ ID NO: 9 or a sequence that is at least 80%, 85%, 90%, or 95% identical to SEQ ID NO: 9) and TPOR BOX1-BOX2 (e.g., SEQ ID NO: 7 or a sequence that is at least 80%, 85%, 90%, or 95% identical to SEQ ID NO: 7), a IL-21R ICD (e.g., SEQ ID NO: 20 or a sequence that is at least 80%, 85%, 90%, or 95% identical to SEQ ID NO: 20) and a IL-15Rβ ICD (e.g., SEQ ID NO: 10 or a sequence that is at least 80%, 85%, 90%, or 95% identical to SEQ ID NO: 10). The cell may comprise an amino acid sequence set forth in SEQ ID NO: 28, or an amino acid sequence that is at least 80%, 85%, 90%, 95% or 99% identical to the amino acid sequence set forth in SEQ ID NO: 28.

The cell may comprise a CAR backbone sequence encoding a CAR backbone polypeptide comprising from the N-terminus to the C-terminus: a signal peptide (e.g., SEQ ID NO: 1 or a sequence that is at least 80%, 85%, 90%, or 95% identical to SEQ ID NO: 1), a GPC3-CAR (e.g., SEQ ID NO: 4 or a sequence that is at least 80%, 85%, 90%, or 95% identical to SEQ ID NO: 4), via a P2A cleavage site (e.g., SEQ ID NO: 5 or a sequence that is at least 80%, 85%, 90%, or 95% identical to SEQ ID NO: 5), and is further connected to a NKG2D chimeric cytokine receptor molecule comprising from the N-terminus to the C-terminus: a signal peptide (e.g., SEQ ID NO: 1 or a sequence that is at least 80%, 85%, 90%, or 95% identical to SEQ ID NO: 1), a NKG2D ECD (e.g., SEQ ID NO: 6 or a sequence that is at least 80%, 85%, 90%, or 95% identical to SEQ ID NO: 6), a TPOR TM (e.g., SEQ ID NO: 9 or a sequence that is at least 80%, 85%, 90%, or 95% identical to SEQ ID NO: 9) and TPOR BOX1-BOX2 (e.g., SEQ ID NO: 7 or a sequence that is at least 80%, 85%, 90%, or 95% identical to SEQ ID NO: 7), a IL-15Rβ ICD (e.g., SEQ ID NO: 10 or a sequence that is at least 80%, 85%, 90%, or 95% identical to SEQ ID NO: 10) and a IL-21R ICD with Y519F mutation (e.g., SEQ ID NO: 21 or a sequence that is at least 80%, 85%, 90%, or 95% identical to SEQ ID NO: 21). The cell may comprise an amino acid sequence set forth in SEQ ID NO: 29, or an amino acid sequence that is at least 80%, 85%, 90%, 95% or 99% identical to the amino acid sequence set forth in SEQ ID NO: 29.

The cell may comprise a CAR backbone sequence encoding a CAR backbone polypeptide comprising from the N-terminus to the C-terminus: a signal peptide (e.g., SEQ ID NO: 1 or a sequence that is at least 80%, 85%, 90%, or 95% identical to SEQ ID NO: 1), a GPC3-CAR (e.g., SEQ ID NO: 4 or a sequence that is at least 80%, 85%, 90%, or 95% identical to SEQ ID NO: 4), via a P2A cleavage site (e.g., SEQ ID NO: 5 or a sequence that is at least 80%, 85%, 90%, or 95% identical to SEQ ID NO: 5), and is further connected to a NKG2D chimeric cytokine receptor molecule comprising from the N-terminus to the C-terminus: a signal peptide (e.g., SEQ ID NO: 1 or a sequence that is at least 80%, 85%, 90%, or 95% identical to SEQ ID NO: 1), a NKG2D ECD (e.g., SEQ ID NO: 6 or a sequence that is at least 80%, 85%, 90%, or 95% identical to SEQ ID NO: 6), a TPOR TM (e.g., SEQ ID NO: 9 or a sequence that is at least 80%, 85%, 90%, or 95% identical to SEQ ID NO: 9) and TPOR BOX1-BOX2 (e.g., SEQ ID NO: 7 or a sequence that is at least 80%, 85%, 90%, or 95% identical to SEQ ID NO: 7), a IL-15Rβ ICD with Y364F mutation (e.g., SEQ ID NO: 11 or a sequence that is at least 80%, 85%, 90%, or 95% identical to SEQ ID NO: 11) and a IL-21R ICD (e.g., SEQ ID NO: 20 or a sequence that is at least 80%, 85%, 90%, or 95% identical to SEQ ID NO: 20). The cell may comprise an amino acid sequence set forth in SEQ ID NO: 30, or an amino acid sequence that is at least 80%, 85%, 90%, 95% or 99% identical to the amino acid sequence set forth in SEQ ID NO: 30.

The cell may comprise a CAR backbone sequence encoding a CAR backbone polypeptide comprising from the N-terminus to the C-terminus: a signal peptide (e.g., SEQ ID NO: 1 or a sequence that is at least 80%, 85%, 90%, or 95% identical to SEQ ID NO: 1), a GPC3-CAR (e.g., SEQ ID NO: 4 or a sequence that is at least 80%, 85%, 90%, or 95% identical to SEQ ID NO: 4), via a P2A cleavage site (e.g., SEQ ID NO: 5 or a sequence that is at least 80%, 85%, 90%, or 95% identical to SEQ ID NO: 5), and is further connected to a NKG2D chimeric cytokine receptor molecule comprising from the N-terminus to the C-terminus: a signal peptide (e.g., SEQ ID NO: 1 or a sequence that is at least 80%, 85%, 90%, or 95% identical to SEQ ID NO: 1), a NKG2D ECD (e.g., SEQ ID NO: 6 or a sequence that is at least 80%, 85%, 90%, or 95% identical to SEQ ID NO: 6), a TPOR TM (e.g., SEQ ID NO: 9 or a sequence that is at least 80%, 85%, 90%, or 95% identical to SEQ ID NO: 9) and TPOR BOX1-BOX2 (e.g., SEQ ID NO: 7 or a sequence that is at least 80%, 85%, 90%, or 95% identical to SEQ ID NO: 7), a IL-15Rβ ICD with Y364F, Y381F, Y384F, Y387F mutation (e.g., SEQ ID NO: 12 or a sequence that is at least 80%, 85%, 90%, or 95% identical to SEQ ID NO: 12) and a IL-21R ICD (e.g., SEQ ID NO: 20 or a sequence that is at least 80%, 85%, 90%, or 95% identical to SEQ ID NO: 20). The cell may comprise an amino acid sequence set forth in SEQ ID NO: 31, or an amino acid sequence that is at least 80%, 85%, 90%, 95% or 99% identical to the amino acid sequence set forth in SEQ ID NO: 31.

The cell may comprise a CAR backbone sequence encoding a CAR backbone polypeptide comprising from the N-terminus to the C-terminus: a signal peptide (e.g., SEQ ID NO: 1 or a sequence that is at least 80%, 85%, 90%, or 95% identical to SEQ ID NO: 1), a GPC3-CAR (e.g., SEQ ID NO: 4 or a sequence that is at least 80%, 85%, 90%, or 95% identical to SEQ ID NO: 4), via a P2A cleavage site (e.g., SEQ ID NO: 5 or a sequence that is at least 80%, 85%, 90%, or 95% identical to SEQ ID NO: 5), and is further connected to a NKG2D chimeric cytokine receptor molecule comprising from the N-terminus to the C-terminus: a signal peptide (e.g., SEQ ID NO: 1 or a sequence that is at least 80%, 85%, 90%, or 95% identical to SEQ ID NO: 1), a NKG2D ECD (e.g., SEQ ID NO: 6 or a sequence that is at least 80%, 85%, 90%, or 95% identical to SEQ ID NO: 6), a TPOR TM (e.g., SEQ ID NO: 9 or a sequence that is at least 80%, 85%, 90%, or 95% identical to SEQ ID NO: 9) and TPOR BOX1-BOX2 (e.g., SEQ ID NO: 7 or a sequence that is at least 80%, 85%, 90%, or 95% identical to SEQ ID NO: 7), a IL-15Rβ ICD with Y418F mutation (e.g., SEQ ID NO: 13 or a sequence that is at least 80%, 85%, 90%, or 95% identical to SEQ ID NO: 13) and a IL-21R ICD (e.g., SEQ ID NO: 20 or a sequence that is at least 80%, 85%, 90%, or 95% identical to SEQ ID NO: 20). The cell may comprise an amino acid sequence set forth in SEQ ID NO: 32, or an amino acid sequence that is at least 80%, 85%, 90%, 95% or 99% identical to the amino acid sequence set forth in SEQ ID NO: 32.

The cell may comprise a CAR backbone sequence encoding a CAR backbone polypeptide comprising from the N-terminus to the C-terminus: a signal peptide (e.g., SEQ ID NO: 1 or a sequence that is at least 80%, 85%, 90%, or 95% identical to SEQ ID NO: 1), a GPC3-CAR (e.g., SEQ ID NO: 4 or a sequence that is at least 80%, 85%, 90%, or 95% identical to SEQ ID NO: 4), via a P2A cleavage site (e.g., SEQ ID NO: 5 or a sequence that is at least 80%, 85%, 90%, or 95% identical to SEQ ID NO: 5), and is further connected to a NKG2D chimeric cytokine receptor molecule comprising from the N-terminus to the C-terminus: a signal peptide (e.g., SEQ ID NO: 1 or a sequence that is at least 80%, 85%, 90%, or 95% identical to SEQ ID NO: 1), a NKG2D ECD (e.g., SEQ ID NO: 6 or a sequence that is at least 80%, 85%, 90%, or 95% identical to SEQ ID NO: 6), a TPOR TM (e.g., SEQ ID NO: 9 or a sequence that is at least 80%, 85%, 90%, or 95% identical to SEQ ID NO: 9) and TPOR BOX1-BOX2 (e.g., SEQ ID NO: 7 or a sequence that is at least 80%, 85%, 90%, or 95% identical to SEQ ID NO: 7), a IL-15Rβ ICD with Y536F mutation (e.g., SEQ ID NO: 14 or a sequence that is at least 80%, 85%, 90%, or 95% identical to SEQ ID NO: 14) and a IL-21R ICD (e.g., SEQ ID NO: 20 or a sequence that is at least 80%, 85%, 90%, or 95% identical to SEQ ID NO: 20). The cell may comprise an amino acid sequence set forth in SEQ ID NO: 33, or an amino acid sequence that is at least 80%, 85%, 90%, 95% or 99% identical to the amino acid sequence set forth in SEQ ID NO: 33.

The cell may comprise a CAR backbone sequence encoding a CAR backbone polypeptide comprising from the N-terminus to the C-terminus: a signal peptide (e.g., SEQ ID NO: 1 or a sequence that is at least 80%, 85%, 90%, or 95% identical to SEQ ID NO: 1), a GPC3-CAR (e.g., SEQ ID NO: 4 or a sequence that is at least 80%, 85%, 90%, or 95% identical to SEQ ID NO: 4), via a P2A cleavage site (e.g., SEQ ID NO: 5 or a sequence that is at least 80%, 85%, 90%, or 95% identical to SEQ ID NO: 5), and is further connected to a NKG2D chimeric cytokine receptor molecule comprising from the N-terminus to the C-terminus: a signal peptide (e.g., SEQ ID NO: 1 or a sequence that is at least 80%, 85%, 90%, or 95% identical to SEQ ID NO: 1), a NKG2D ECD (e.g., SEQ ID NO: 6 or a sequence that is at least 80%, 85%, 90%, or 95% identical to SEQ ID NO: 6), a TPOR TM (e.g., SEQ ID NO: 9 or a sequence that is at least 80%, 85%, 90%, or 95% identical to SEQ ID NO: 9) and TPOR BOX1-BOX2 (e.g., SEQ ID NO: 7 or a sequence that is at least 80%, 85%, 90%, or 95% identical to SEQ ID NO: 7), a IL-15Rβ ICD with Y418F, Y536F mutation (e.g., SEQ ID NO: 15 or a sequence that is at least 80%, 85%, 90%, or 95% identical to SEQ ID NO: 15) and a IL-21R ICD (e.g., SEQ ID NO: 20 or a sequence that is at least 80%, 85%, 90%, or 95% identical to SEQ ID NO: 20). The cell may comprise an amino acid sequence set forth in SEQ ID NO: 34, or an amino acid sequence that is at least 80%, 85%, 90%, 95% or 99% identical to the amino acid sequence set forth in SEQ ID NO: 34.

The cell may comprise a CAR backbone sequence encoding a CAR backbone polypeptide comprising from the N-terminus to the C-terminus: a signal peptide (e.g., SEQ ID NO: 1 or a sequence that is at least 80%, 85%, 90%, or 95% identical to SEQ ID NO: 1), a GPC3-CAR (e.g., SEQ ID NO: 4 or a sequence that is at least 80%, 85%, 90%, or 95% identical to SEQ ID NO: 4), via a P2A cleavage site (e.g., SEQ ID NO: 5 or a sequence that is at least 80%, 85%, 90%, or 95% identical to SEQ ID NO: 5), and is further connected to a NKG2D chimeric cytokine receptor molecule comprising from the N-terminus to the C-terminus: a signal peptide (e.g., SEQ ID NO: 1 or a sequence that is at least 80%, 85%, 90%, or 95% identical to SEQ ID NO: 1), a NKG2D ECD (e.g., SEQ ID NO: 6 or a sequence that is at least 80%, 85%, 90%, or 95% identical to SEQ ID NO: 6), a TPOR TM (e.g., SEQ ID NO: 9 or a sequence that is at least 80%, 85%, 90%, or 95% identical to SEQ ID NO: 9) and TPOR BOX1-BOX2 (e.g., SEQ ID NO: 7 or a sequence that is at least 80%, 85%, 90%, or 95% identical to SEQ ID NO: 7), a IL-15Rβ ICD with Y364F, Y381F, Y384F, Y387F, Y418F, Y536F mutation (e.g., SEQ ID NO: 16 or a sequence that is at least 80%, 85%, 90%, or 95% identical to SEQ ID NO: 16) and a IL-21R ICD (e.g., SEQ ID NO: 20 or a sequence that is at least 80%, 85%, 90%, or 95% identical to SEQ ID NO: 20). The cell may comprise an amino acid sequence set forth in SEQ ID NO: 35, or an amino acid sequence that is at least 80%, 85%, 90%, 95% or 99% identical to the amino acid sequence set forth in SEQ ID NO: 35.

The cell may comprise a CAR backbone sequence encoding a CAR backbone polypeptide comprising from the N-terminus to the C-terminus: a signal peptide (e.g., SEQ ID NO: 1 or a sequence that is at least 80%, 85%, 90%, or 95% identical to SEQ ID NO: 1), a GPC3-CAR (e.g., SEQ ID NO: 4 or a sequence that is at least 80%, 85%, 90%, or 95% identical to SEQ ID NO: 4), via a P2A cleavage site (e.g., SEQ ID NO: 5 or a sequence that is at least 80%, 85%, 90%, or 95% identical to SEQ ID NO: 5), and is further connected to a NKG2D chimeric cytokine receptor molecule comprising from the N-terminus to the C-terminus: a signal peptide (e.g., SEQ ID NO: 1 or a sequence that is at least 80%, 85%, 90%, or 95% identical to SEQ ID NO: 1), a NKG2D ECD (e.g., SEQ ID NO: 6 or a sequence that is at least 80%, 85%, 90%, or 95% identical to SEQ ID NO: 6), a TPOR TM (e.g., SEQ ID NO: 9 or a sequence that is at least 80%, 85%, 90%, or 95% identical to SEQ ID NO: 9) and TPOR BOX1-BOX2 (e.g., SEQ ID NO: 7 or a sequence that is at least 80%, 85%, 90%, or 95% identical to SEQ ID NO: 7), a IL-15Rβ truncated1 345-431 ICD (e.g., SEQ ID NO: 17 or a sequence that is at least 80%, 85%, 90%, or 95% identical to SEQ ID NO: 17) and a IL-21R ICD (e.g., SEQ ID NO: 20 or a sequence that is at least 80%, 85%, 90%, or 95% identical to SEQ ID NO: 20). The cell may comprise an amino acid sequence set forth in SEQ ID NO: 36, or an amino acid sequence that is at least 80%, 85%, 90%, 95% or 99% identical to the amino acid sequence set forth in SEQ ID NO: 36.

The cell may comprise a CAR backbone sequence encoding a CAR backbone polypeptide comprising from the N-terminus to the C-terminus: a signal peptide (e.g., SEQ ID NO: 1 or a sequence that is at least 80%, 85%, 90%, or 95% identical to SEQ ID NO: 1), a GPC3-CAR (e.g., SEQ ID NO: 4 or a sequence that is at least 80%, 85%, 90%, or 95% identical to SEQ ID NO: 4), via a P2A cleavage site (e.g., SEQ ID NO: 5 or a sequence that is at least 80%, 85%, 90%, or 95% identical to SEQ ID NO: 5), and is further connected to a NKG2D chimeric cytokine receptor molecule comprising from the N-terminus to the C-terminus: a signal peptide (e.g., SEQ ID NO: 1 or a sequence that is at least 80%, 85%, 90%, or 95% identical to SEQ ID NO: 1), a NKG2D ECD (e.g., SEQ ID NO: 6 or a sequence that is at least 80%, 85%, 90%, or 95% identical to SEQ ID NO: 6), a TPOR TM (e.g., SEQ ID NO: 9 or a sequence that is at least 80%, 85%, 90%, or 95% identical to SEQ ID NO: 9) and TPOR BOX1-BOX2 (e.g., SEQ ID NO: 7 or a sequence that is at least 80%, 85%, 90%, or 95% identical to SEQ ID NO: 7), a IL-15Rβ truncated2 345-404 ICD (e.g., SEQ ID NO: 18 or a sequence that is at least 80%, 85%, 90%, or 95% identical to SEQ ID NO: 18) and a IL-21R ICD (e.g., SEQ ID NO: 20 or a sequence that is at least 80%, 85%, 90%, or 95% identical to SEQ ID NO: 20). The cell may comprise an amino acid sequence set forth in SEQ ID NO: 37, or an amino acid sequence that is at least 80%, 85%, 90%, 95% or 99% identical to the amino acid sequence set forth in SEQ ID NO: 37.

The cell may comprise a CAR backbone sequence encoding a CAR backbone polypeptide comprising from the N-terminus to the C-terminus: a signal peptide (e.g., SEQ ID NO: 1 or a sequence that is at least 80%, 85%, 90%, or 95% identical to SEQ ID NO: 1), a GPC3-CAR (e.g., SEQ ID NO: 91 or a sequence that is at least 80%, 85%, 90%, or 95% identical to SEQ ID NO: 91), via a P2A cleavage site (e.g., SEQ ID NO: 5 or a sequence that is at least 80%, 85%, 90%, or 95% identical to SEQ ID NO: 5), and is further connected to a NKG2D chimeric cytokine receptor molecule comprising from the N-terminus to the C-terminus: a signal peptide (e.g., SEQ ID NO: 1 or a sequence that is at least 80%, 85%, 90%, or 95% identical to SEQ ID NO: 1), a NKG2D ECD (e.g., SEQ ID NO: 6 or a sequence that is at least 80%, 85%, 90%, or 95% identical to SEQ ID NO: 6), a TPOR transmembrane (TM) (e.g., SEQ ID NO: 9 or a sequence that is at least 80%, 85%, 90%, or 95% identical to SEQ ID NO: 9) and TPOR BOX1-BOX2 (e.g., SEQ ID NO: 7 or a sequence that is at least 80%, 85%, 90%, or 95% identical to SEQ ID NO: 7), a IL-15Rβ ICD (e.g., SEQ ID NO: 10 or a sequence that is at least 80%, 85%, 90%, or 95% identical to SEQ ID NO: 10) and a IL-21R ICD (e.g., SEQ ID NO: 20 or a sequence that is at least 80%, 85%, 90%, or 95% identical to SEQ ID NO: 20). The cell may comprise an amino acid sequence set forth in SEQ ID NO: 92, or an amino acid sequence that is at least 80%, 85%, 90%, 95% or 99% identical to the amino acid sequence set forth in SEQ ID NO: 92.

The cell may comprise a CAR backbone sequence encoding a CAR backbone polypeptide comprising from the N-terminus to the C-terminus: a signal peptide (e.g., SEQ ID NO: 1 or a sequence that is at least 80%, 85%, 90%, or 95% identical to SEQ ID NO: 1), a GPC3-CAR (e.g., SEQ ID NO: 91 or a sequence that is at least 80%, 85%, 90%, or 95% identical to SEQ ID NO: 91), via a P2A cleavage site (e.g., SEQ ID NO: 5 or a sequence that is at least 80%, 85%, 90%, or 95% identical to SEQ ID NO: 5), and is further connected to a NKG2D chimeric cytokine receptor molecule comprising from the N-terminus to the C-terminus: a signal peptide (e.g., SEQ ID NO: 1 or a sequence that is at least 80%, 85%, 90%, or 95% identical to SEQ ID NO: 1), a NKG2D ECD (e.g., SEQ ID NO: 6 or a sequence that is at least 80%, 85%, 90%, or 95% identical to SEQ ID NO: 6), a CD28 TM (e.g., SEQ ID NO: 3 or a sequence that is at least 80%, 85%, 90%, or 95% identical to SEQ ID NO: 3), a TPOR-BOX1 (e.g., SEQ ID NO: 8 or a sequence that is at least 80%, 85%, 90%, or 95% identical to SEQ ID NO: 8), a IL-15Rβ ICD (e.g., SEQ ID NO: 10 or a sequence that is at least 80%, 85%, 90%, or 95% identical to SEQ ID NO: 10) and a IL-21R ICD (e.g., SEQ ID NO: 20 or a sequence that is at least 80%, 85%, 90%, or 95% identical to SEQ ID NO: 20). The cell may comprise an amino acid sequence set forth in SEQ ID NO: 93, or an amino acid sequence that is at least 80%, 85%, 90%, 95% or 99% identical to the amino acid sequence set forth in SEQ ID NO: 93.

The cell that is engineered may be obtained from, e.g., humans and non-human animals. The cell that is engineered may be obtained from bacteria, fungi, humans, rats, mice, rabbits, monkeys, pig or any other species. The cell may be from humans, rats or mice. The cells may be mouse lymphocytes and engineered (e.g., transduced) to express the chimeric cytokine receptor and CAR described herein. The cell may be obtained from humans. The cell that is engineered may be a blood cell. The cell may be a CD8+ T cell, a CD4+ T cell, a memory T cell, a Treg cell, a natural killer (NK) cell, a natural killer T (NKT) cell, a B cell, or a macrophage/monocyte.

The preparation of the engineered cells may include one or more culture and/or preparation steps. The cells for introduction of the binding molecule, e.g., CAR, can be isolated from a sample, such as a biological sample, e.g., one obtained from or derived from a subject. The subject from which the cell is isolated may be one having the disease or condition or in need of a cell therapy or to which cell therapy will be administered. The subject may be a human in need of a particular therapeutic intervention, such as the adoptive cell therapy for which cells are being isolated, processed, and/or engineered.

The cell may be an NK cell. Human natural killer cells (NK cells) play an important role in innate immune defense against malignant lymphoma cells, and thus are suitable for adoptive immune therapy (i.e., adoptive cellular immunotherapy). However, due to difficulties in ex vivo cell expansion and differences in activities of NK cells in individual patients, it is difficult to use the NK cells.

NK cells are part of the innate immune system, providing the first line of defense against pathogens and cancer cells. They produce cytokines and mediate cytotoxicity without the need for prior sensitization and have the ability to interact with, and activate other immune cells. NK cells for immunotherapy can be generated from multiple sources, such as expanded autologous or allogeneic peripheral blood, umbilical cord blood, hematopoietic stem cells, induced pluripotent stem cells, as well as cell lines.

NK cells activation and effector function is a complex process as it depends upon the integration of signals from two distinct types of receptors-activating and inhibitory receptors. Normal healthy cells express MHC class I molecules on their surface, which act as ligands for inhibitory receptors and contribute to self-tolerance of NK cells. Cellular stress associated with viral infection or tumor development such as DNA damage, senescence or tumor suppressor genes upregulate ligands for activating receptors. This results in shift of balance to NK cells activation. Transmembrane and cytoplasmic stimulatory/activator molecules in NK cells can affect NK cell differentiation pathways, metabolic cycles, apoptosis as well as activation induced cell death.

The cell may be a T cell. The T cells can express a cell surface receptor that recognizes a specific antigenic moiety on the surface of a target cell. The cell surface receptor can be a wild type or recombinant T cell receptor (TCR), a chimeric antigen receptor (CAR), or any other surface receptor capable of recognizing an antigenic moiety that is associated with the target cell. T cells can be obtained by various methods known in the art, e.g., in vitro culture of T cells (e.g., tumor infiltrating lymphocytes) isolated from patients. Genetically modified T cells can be obtained by transducing T cells (e.g., isolated from the peripheral blood of patients), with a viral vector. The T cells can be CD4+ T cells, CD8+ T cells, or regulatory T cells. The T cells can be T helper type 1 T cells and T helper type 2 T cells. The T cell expressing this receptor may be an αβT cell. The T cell expressing this receptor may be a γδT cell. The T cells may be central memory T cells. The T cells may be effector memory T cells. The T cells may be naïve T cells. The T cells may be a combination of αβT cell and γβT cell.

The cells may be stem cells, such as multipotent and pluripotent stem cells, including induced pluripotent stem cells (iPSCs). The cells can be primary cells, such as those isolated directly from a subject and/or isolated from a subject and frozen. The stem cells may be cultured with additional differentiation factors to obtain desired cell types (e.g., NK cells).

Different cell types can be obtained from appropriate isolation methods. The isolation methods include the separation of different cell types based on the expression or presence in the cell of one or more specific molecules, such as surface markers, e.g., surface proteins, intracellular markers, or nucleic acid. Any known method for separation based on such markers can be used. The separation can be affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. Negative selection can be particularly useful where no antibody is available that specifically identifies a cell type in a heterogeneous population, such that separation is best carried out based on markers expressed by cells other than the desired population.

Also provided are populations of cells (e.g., genetically engineered cells), compositions containing such cells and/or enriched for such cells, such as in which cells expressing the binding molecule make up at least 15%, 20%, 25%, 30%, 35%, 40%, 50%, 60%, 70%, 80%, 90%, 91%, 92%, 93%, 94%, 95%, 96%, 97%, 98%, 99%, or more percent of the total cells in the composition or cells of a certain type such as NK cells, T cells, CD8+ or CD4+ cells.

The engineered cells (e.g. cells expressing the chimeric cytokine receptor and the CAR described herein) are co-cultured with target cells (e.g., cells expressing antigens) for at least or about 1 hour, 2 hours, 3 hours, 4 hours, 5 hours, 6 hours, 12 hours, 18 hours, 1 day, 2 days, 3 days, or longer, such that the engineered cells (e.g., cells expressing the chimeric cytokine receptor and the CAR described herein) can be activated.

The engineered cells (e.g., cells expressing the chimeric cytokine receptor and the CAR described herein) may have a cytotoxicity effect on tumor cells. The engineered cells (e.g., cells expressing the chimeric cytokine receptor and the CAR described herein) may kill about 10%, about 20%, about 30%, about 40%, about 50%, about 60%, about 70%, about 80%, about 90% or more tumor cells when contacted with the tumor cells. The engineered cells (e.g., cells expressing the chimeric cytokine receptor and the CAR described herein) may be contacted with tumor cells at a ratio of about 1:1, 1:2, 1:3, 1:4, 1:5, 1:6, 1:7, 1:8, 1:9, 1:10, 10:1, 9:1, 8:1, 7:1, 6:1, 5:1, 4:1, 3:1, or 2:1.

The engineered cells (e.g., cells expressing the chimeric cytokine receptor and the CAR described herein) may be contacted with the tumor cells for about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days or more.

The engineered cells (e.g., cells expressing the chimeric cytokine receptor and the CAR described herein) may induce the production of one or more cytokines after contacted with the target cells. The cytokine may be interferon γ (IFN-γ). The cytokine may be granulocyte-macrophage colony-stimulating factor (GM-CSF). The engineered cells (e.g., cells expressing the chimeric cytokine receptor and the CAR described herein) may induce less cytokine production than cells without the expression of the chimeric cytokine receptor and/or the CAR. The engineered cells (e.g., cells expressing the chimeric cytokine receptor and the CAR described herein) may induce more cytokine production than cells without the expression of the chimeric cytokine receptor and/or the CAR.

The engineered cells (e.g., cells expressing the chimeric cytokine receptor and the CAR described herein) may reduce or slow down tumor progression in a subject having cancer. The progression of the cancer may be evaluated every 1, 2, 3, 4, 5, 6, or 7 days. The progression of the cancer may be evaluated every 1, 2, 3, 4 weeks. The progression of the cancer may be evaluated every 1, 2, 3, 4, 5, 6, 7, 8, 9, 10, 11, 12 or more months. The engineered cells (e.g., cells expressing the chimeric cytokine receptor and the CAR described herein) may retain therapeutic activity, efficacy and persistence after about 1, 2, 3, 4, 5, 6, or 7 days. The engineered cells (e.g., cells expressing the chimeric cytokine receptor and the CAR described herein) may retain therapeutic activity, efficacy and persistence after about 1, 2, 3, 4 weeks. The engineered cells (e.g., cells expressing the chimeric cytokine receptor and the CAR described herein) may retain therapeutic activity, efficacy and persistence after about 1, 2, 3, 4, 5, 6, 7, 8, 9, 10, 11, 12 or more months. About 5%, 10%, 15%, 20%, 25%, 30%, 35%, 40%, 45%, 50%, 55%, 60%, 65%, 70%, 75%, 80%, 85%, 90%, 95% or more of the engineered cells (e.g., cells expressing the chimeric cytokine receptor and the CAR described herein) may be retained after about 1, 2, 3, 4, 5, 6, or 7 days, about 1, 2, 3, 4 weeks, or about 1, 2, 3, 4, 5, 6, 7, 8, 9, 10, 11, 12 or more months.

The engineered cells (e.g., cells expressing the chimeric cytokine receptor and the CAR described herein) may retain proliferation/expansion for about 10⁰, 10¹, 10², 10³, 10⁴, 10⁵, 10⁶, 10⁷, 10⁸, 10⁹, 10¹⁰ or more folds in long-term co-cultures. The engineered cells (e.g., cells expressing the chimeric cytokine receptor and the CAR described herein) may retain proliferation/expansion after about 1, 2, 3, 4, 5, 6, 7, 8, 9, 10 or more rechallenges in long-term co-cultures.

The engineered cells (e.g., cells expressing the chimeric cytokine receptor and the CAR described herein) may be able to secrete one or more cytokines, e.g., IFN-γ about 1, 2, 3, 4, or more weeks after the administration. The engineered cells (e.g., cells expressing the chimeric cytokine receptor and the CAR described herein) may be able to secrete one or more cytokines, e.g., IFN-γ at a level of about 0.1-50000 pg/mL, about 1-50000 pg/mL, about 10-50000 pg/mL, about 100-50000 pg/mL, about 1000-50000 pg/mL, about 10000-50000 pg/mL, 0.1-10000 pg/mL, about 1-10000 pg/mL, about 10-10000 pg/mL, about 100-10000 pg/mL, or about 1000-10000 pg/mL.

### Nucleic Acids

The present disclosure provides nucleic acids comprising a nucleic acid sequence encoding the chimeric cytokine receptor described herein. The nucleic acid may further comprise a nucleic acid sequence encoding a functional exogenous receptor described herein or a fusion protein comprising the functional exogenous receptor and the chimeric cytokine receptor described herein.

A polynucleotide of the present disclosure may comprise a first polynucleotide sequence and a second polynucleotide sequence. The first and second polynucleotide sequence can be separated by a linker. A linker for use in the present disclosure allows for multiple proteins to be encoded by the same nucleic acid sequence (e.g., a multicistronic or bicistronic sequence), which are translated as a polyprotein that is dissociated into separate protein components. The polynucleotide may comprises from 5′ to 3′ the first polynucleotide sequence, the linker, and the second polynucleotide sequence. The polynucleotide comprises from 5′ to 3′ the second polynucleotide sequence, the linker, and the first polynucleotide sequence. The first polynucleotide sequence may encode a functional exogenous receptor (e.g., CAR) described herein and the second polynucleotide sequence may encode a chimeric cytokine receptor described herein. The first polynucleotide sequence may encode a chimeric cytokine receptor described herein and the second polynucleotide sequence may encode a functional exogenous receptor (e.g., CAR) described herein.

The linker may comprise a nucleic acid sequence that encodes for an internal ribosome entry site (IRES). As used herein, “an internal ribosome entry site” or “IRES” refers to an element that promotes direct internal ribosome entry to the initiation codon, such as ATG, of a protein coding region, thereby leading to cap-independent translation of the gene. Various internal ribosome entry sites are known to those of skill in the art, including, without limitation, IRES obtainable from viral or cellular mRNA sources, e.g., immunoglobulin heavy-chain binding protein (BiP); vascular endothelial growth factor (VEGF); fibroblast growth factor 2; insulin-like growth factor; translational initiation factor eIF4G; yeast transcription factors TFIID and HAP4; and IRES obtainable from, e.g., cardiovirus, rhinovirus, aphthovirus, HCV, Friend murine leukemia virus (FrMLV), and Moloney murine leukemia virus (MoMLV). Those of skill in the art would be able to select the appropriate IRES.

The linker may comprise a nucleic acid sequence that encodes for a self-cleaving peptide. As used herein, a “self-cleaving peptide” or “2A peptide” refers to an oligopeptide that allow multiple proteins to be encoded as polyproteins, which dissociate into component proteins upon translation. Use of the term “self-cleaving” is not intended to imply a proteolytic cleavage reaction. Various self-cleaving or 2A peptides are known to those of skill in the art, including, without limitation, those found in members of the Picornaviridae virus family, e.g., foot-and-mouth disease virus (FMDV), equine rhinitis A virus (ERAV0, Thosea asigna virus (TaV), and porcine tescho virus-1 (PTV-1); and carioviruses such as Theilovirus and encephalomyocarditis viruses.2A peptides derived from FMDV, ERAV, PTV-1, and TaV are referred to herein as “F2A,” “E2A,” “P2A,” and “T2A,” respectively. Those of skill in the art would be able to select the appropriate self-cleaving peptide.

The linker peptide can comprise at least or about 5, 6, 7, 8, 9, 10, 11, 12, 13, 14, 15, 16, 17, 18, 19, 20, 30, 40, or 50 amino acid residues. The linker peptide may comprise at least or about 4, 5, 6, 7, 8, 9, 10, 11, 12, 13, 14, 15, 16, 20, 25, 30, or 40 glycine residues. The linker peptide may comprise at least or about 1, 2, 3, 4, 5, 6, 7, or 8 serine residues. The linker peptide may comprise or consists of both glycine and serine residues. The linker sequence may have no more than 10, 11, 12, 13, 14, 15, 16, 17, 18, 19, 20, 30, 40, or 50 amino acid residues. The linker peptide may comprise 1, 2, 3, 4, or 5 amino acid insertions, deletions, or substitutions.

The linker can comprise a spacer sequence. Various spacer sequences are known in the art, including, without limitation, glycine serine (GS) spacers (also known as GS linkers) such as (GS)n, (SG)n, (GSGGS)n (SEQ ID NO: 84) and (GGGS)n (SEQ ID NO: 85), where n represents an integer of at least 1. Those of skill in the art would be able to select the appropriate spacer sequence.

A polynucleotide of the present disclosure can be operably linked to a transcriptional control element, e.g., a promoter, and enhancer, etc. Suitable promoter and enhancer elements are known to those of skill in the art.

The promoter may be a CD8 cell-specific promoter, a CD4 cell-specific promoter, a neutrophil-specific promoter, or an NK-specific promoter. For example, a CD4 gene promoter can be used; see, e.g., Salmon et al. Proc. Natl. Acad. Sci. USA (1993) 90:7739; and Marodon et al. (2003) Blood 101:3416. As another example, a CD8 gene promoter can be used. NK cell-specific expression can be achieved by use of an NcrI (p46) promoter; see, e.g., Eckelhart et al. Blood (2011) 117:1565.

Other examples of suitable promoters include the immediate early cytomegalovirus (CMV) promoter sequence. This promoter sequence is a strong constitutive promoter sequence capable of driving high levels of expression of any polynucleotide sequence operatively linked thereto. Other constitutive promoter sequences can also be used, including, but not limited to a simian virus 40 (SV40) early promoter, a mouse mammary tumor virus (MMTV) or human immunodeficiency virus (HIV) long terminal repeat (LTR) promoter, a MoMuLV promoter, an avian leukemia virus promoter, an Epstein-Barr virus immediate early promoter, a Rous sarcoma virus promoter, the EF-1 alpha promoter, as well as human gene promoters such as, but not limited to, an actin promoter, a myosin promoter, a hemoglobin promoter, and a creatine kinase promoter. Further, the disclosure should not be limited to the use of constitutive promoters. Inducible promoters are also contemplated as part of the disclosure. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

An expression vector (e.g., a lentiviral vector) can be used to introduce the chimeric cytokine receptor (CCR), CAR or TCR into an immune cell or precursor thereof (e.g., a T cell). Accordingly, an expression vector (e.g., a lentiviral vector) of the present disclosure can comprise a polynucleotide encoding for a CAR or a TCR. The expression vector (e.g., lentiviral vector) can comprise additional elements that will aid in the functional expression of the CCR or CAR encoded therein. An expression vector comprising a polynucleotide encoding for a CCR or CAR may further comprise a mammalian promoter. The vector may comprise an elongation-factor-1-alpha promoter (EF-1α promoter). The use of an EF-1α promoter can increase the efficiency in expression of downstream transgenes (e.g., a CCR- or CAR-encoding polynucleotide). Physiologic promoters (e.g., an EF-1α promoter) can be less likely to induce integration mediated genotoxicity, and can abrogate the ability of the retroviral vector to transform stem cells. Other physiological promoters suitable for use in a vector (e.g., lentiviral vector) are known to those of skill in the art and can be incorporated into a vector of the present disclosure. The vector (e.g., lentiviral vector) may further comprise a non-requisite cis acting sequence that can improve titers and gene expression.

The polynucleotide may encode an amino acid sequence set forth in any one of SEQ ID NOs: 22-37, 92 and 93, or an amino acid sequence that is at least 80%, 85%, 90%, 95% or 99% identical to the amino acid sequence set forth in any one of SEQ ID NOs: 22-37, 92 and 93.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some cases, the disclosure relates to nucleotide sequences encoding any peptides that are described herein, or any amino acid sequences that are encoded by any nucleotide sequences as described herein. The nucleic acid sequence may be less than 10, 20, 30, 40, 50, 60, 70, 80, 90, 100, 110, 120, 130, 150, 200, 250, 300, 350, 400, 500, 600, 800, 1000, 1200, 1400, 1600, 1800, 2000, 2500, 3000, 3500, 4000, or 5000 nucleotides. The amino acid sequence may be less than 5, 6, 7, 8, 9, 10, 20, 30, 40, 50, 60, 70, 80, 90, 100, 110, 120, 130, 140, 150, 160, 170, 180, 190, 200, 220, 240, 260, 280, 300, 350, 400, 450, 500, 550, 600, 700, 800, 900, 1000, 1100, 1200, 1300, or 1400 amino acid residues.

The amino acid sequence may (i) comprise an amino acid sequence; or (ii) consist of an amino acid sequence, wherein the amino acid sequence is any one of the sequences as described herein.

The nucleic acid sequence may (i) comprise a nucleic acid sequence; or (ii) consist of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes may be at least 80% of the length of the reference sequence, and may be at least 90%, 95%, or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

Introduction of Polynucleotides into Host Cells

The polynucleotides (e.g., vectors) described herein can be introduced as one or more polynucleotides or constructs, optionally comprising a marker that will allow for selection of host cells that contain the construct(s). The genes and regulatory regions can be isolated, as appropriate, ligated, cloned in an appropriate cloning host, analyzed by restriction or sequencing. Particularly, using PCR, individual fragments including all or portions of a functional unit can be isolated, where one or more mutations can be introduced using “primer repair”, ligation, in vitro mutagensis, etc. as appropriate. The polynucleotides obtained and demonstrated to have the appropriate sequences can then be introduced into the host cell by any convenient means. The polynucleotides can be integrated and packaged into non-replicating, defective viral genomes like lentivirus, Adenovirus, Adeno-associated virus (AAV), or Herpes simplex virus (HSV) or others, including retroviral vectors, for infection or transduction into cells. The polynucleotides can include viral sequences for transfection, if desired. Alternatively, the polynucleotides can be introduced by fusion, electroporation, biolistics, transfection, lipofection, or the like. The host cells can be grown and expanded in culture before introduction of the construct(s), followed by the appropriate treatment for introduction of the construct(s) and integration of the construct(s). The cells are then expanded and screened by virtue of a marker present in the construct. Various markers that can be used successfully include hprt, neomycin resistance, thymidine kinase, hygromycin resistance, etc.

A construct encoding both a chimeric cytokine receptor (CCR) peptide and a CAR can be introduced into the host cell using a lentiviral delivery system. A construct encoding both a CCR peptide and a CAR can be introduced into the host cell using a retroviral delivery system.

The host cells can be human cells. The host cells can be human T cells. The human T cells can be purified from commercialized PBMCs. The host cells can be αβT cells. The host cells can be γδT cells. The host cells can be a combination of αβT and γδT cells. The host cells can be NK cells.

### Methods of Treatment

The chimeric cytokine receptor described herein, the polynucleotides described herein and the modified cells described herein can be used in a variety of experimental, therapeutic and commercial applications.

In one aspect, the disclosure provides a pharmaceutical composition, comprising the chimeric cytokine receptor described herein, the cell described herein, or the nucleic acid described herein and a pharmaceutically acceptable carrier.

In one aspect, the disclosure provides a method of treating a disease or disorder in a subject, comprising administering to the subject an effective amount of the pharmaceutical composition described herein.

In one aspect, the disclosure provides a method of modulating an immune response comprising administering an effective amount of modified cells described herein to a subject in need thereof.

The disease or disorder may be a cancer, an inflammatory or autoimmune disease.

The cancer may be solid cancer or hematologic cancer. The cancer may be liver cancer, gastric cancer, colon cancer, lymphoma, acute myeloid leukemia (AML) or chronic myelogenous leukemia (CML).

The term “effective amount” as used herein means an amount effective, at dosages and for periods of time necessary to achieve the desired results.

Examples of cancer that can be treated include, but are not limited to, leukemias including chronic lymphocytic leukemia, chronic myelogenous leukemia, acute myelogenous leukemia, acute lymphoblastic leukemia, and T cell and B cell leukemias, lymphomas (Hodgkin's and non-Hodgkins), lymphoproliferative disorders, plasmacytomas, histiocytomas, melanomas, adenomas, sarcomas, carcinomas of solid tissues, hypoxic tumors, squamous cell carcinomas, genitourinary cancers such as cervical and bladder cancer, hematopoietic cancers, head and neck cancers, and nervous system cancers.

The disclosure further includes the use of the modified cells described herein in the manufacture of a medicament or pharmaceutical composition to treat a disease or disorder or modulate an immune response, to treat an infection or to treat cancer as described hereinabove.

The modified cells can also be used in experimental models, for example, to further study and elucidate the function of the cells.

One or more of the modified cells described herein can be administered to a subject in a single, unified form, such as an intravenous injection, or in multiple forms, for example, as multiple intravenous infusions or injections, or subcutaneous injections. In some cases, the modified cells can expand within a subject's body, in vivo, after administration to a subject. The modified cells can be frozen to provide cells for multiple treatments with the same cell preparation. The modified cells of the disclosure, and pharmaceutical compositions comprising the same, can be packaged as a kit. A kit can include instructions (e.g., written instructions) on the use of the modified cells and compositions comprising the same.

In one aspect, the present disclosure provides a method of treatment that comprises administering to a subject a therapeutically-effective amount of the modified cells. The therapeutically-effective amount of the modified cells may be administered for at least 1 week, 2 weeks, 3 weeks, 1 month, 2 months, 3 months, 4 months, 5 months, 6 months, or 1 year. The therapeutically-effective amount of the modified cells may be administered for at least one week. The therapeutically-effective amount of the modified cells may be administered for at least two weeks.

The modified cells described herein can be administered before, during, or after the occurrence of a disease or condition, and the timing of administering the modified cells can vary. For example, the modified cells can be used as a prophylactic and can be administered continuously to subjects with a propensity to conditions or diseases in order to lessen a likelihood of the occurrence of the disease or condition. The modified cells can be administered to a subject during or as soon as possible after the onset of the symptoms. The administration of the modified cells can be initiated immediately within the onset of symptoms, within the first 3 hours of the onset of the symptoms, within the first 6 hours of the onset of the symptoms, within the first 24 hours of the onset of the symptoms, within 48 hours of the onset of the symptoms, or within any period of time from the onset of symptoms. The initial administration can be via any route practical (e.g., intravenous infusions or injections), such as by any route described herein using any formulation described herein. In some examples, the administration of the modified cells of the disclosure is an intravenous administration. One or multiple dosages of the modified cells can be administered as soon as is practicable after the onset of a cancer or an infectious disease, and for a length of time necessary for the treatment of the disease, such as, for example, from about 24 hours to about 48 hours, from about 48 hours to about 1 week, from about 1 week to about 2 weeks, from about 2 weeks to about 1 month, from about 1 month to about 3 months. For the treatment of cancer, one or multiple dosages of the modified cells can be administered years after onset of the cancer and before or after other treatments. In some examples, the modified cells can be administered for at least about 10 minutes, 30 minutes, 1 hour, 2 hours, 3 hours, 4 hours, 5 hours, 6 hours, 12 hours, 24 hours, at least 48 hours, at least 72 hours, at least 96 hours, at least 1 week, at least 2 weeks, at least 3 weeks, at least 4 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 1 year, at least 2 years at least 3 years, at least 4 years, or at least 5 years. The length of treatment can vary for each subject.

Methods for administration of modified cells for adoptive cell therapy are known and can be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8 (10): 577-85). See, e.g., Themeli et al. (2013) Nat Biotechnol. 31 (10): 928-933; Tsukahara et al. (2013) Biochem Biophys Res Commun 438 (1): 84-9; Davila et al. (2013) PLOS ONE 8 (4): e61338. The cell therapy, e.g., adoptive T cell therapy may be carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

The cell therapy (e.g., adoptive T cell therapy) may be carried out by allogeneic transfer, in which the cells are isolated and/or otherwise prepared from a subject other than a subject who is to receive or who ultimately receives the cell therapy, e.g., a first subject. In such cases, the cells then are administered to a different subject, e.g., a second subject, of the same species. The first and second subjects may be genetically identical. The first and second subjects may be genetically similar. The second subject may express the same HLA class or supertype as the first subject.

The subject may have been treated with a therapeutic agent targeting the disease or condition, e.g. the tumor, prior to administration of the cells or composition containing the cells. In some aspects, the subject is refractory or non-responsive to the other therapeutic agent. The subject may have persistent or relapsed disease, e.g., following treatment with another therapeutic intervention, including chemotherapy, radiation, and/or hematopoietic stem cell transplantation (HSCT), e.g., allogenic HSCT. The administration may effectively treat the subject despite the subject having become resistant to another therapy.

The subject may be responsive to the other therapeutic agent, and treatment with the therapeutic agent reduces disease burden. In some aspects, the subject is initially responsive to the therapeutic agent, but exhibits a relapse of the disease or condition over time. The subject may not have relapsed. In some cases, the subject is determined to be at risk for relapse, such as at a high risk of relapse, and thus the cells are administered prophylactically, e.g., to reduce the likelihood of or prevent relapse. In some aspects, the subject has not received prior treatment with another therapeutic agent.

The subject may have persistent or relapsed disease, e.g., following treatment with another therapeutic intervention, including chemotherapy, radiation, and/or hematopoietic stem cell transplantation (HSCT), e.g., allogenic HSCT. The administration may effectively treat the subject despite the subject having become resistant to another therapy.

The modified cells described herein can be administered to an animal, optionally a mammal, optionally a human, to treat a cancer. In addition, the modified cells can be used for the treatment of any condition related to a cancer, especially a cell-mediated immune response against a tumor cell(s), where it is desirable to treat or alleviate the disease. The types of cancers to be treated with the modified cells or pharmaceutical compositions include, carcinoma, blastoma, and sarcoma, and certain leukemia or lymphoid malignancies, benign and malignant tumors, and malignancies e.g., sarcomas, carcinomas, and melanomas. Other exemplary cancers include but are not limited breast cancer, prostate cancer, ovarian cancer, cervical cancer, skin cancer, pancreatic cancer, colorectal cancer, renal cancer, liver cancer, brain cancer, lymphoma, leukemia, lung cancer, thyroid cancer, and the like. The cancers can be non-solid tumors (such as hematological tumors) or solid tumors. Adult tumors/cancers and pediatric tumors/cancers are also included. The cancer can be a solid tumor or a hematological tumor. The cancer can be a carcinoma. The cancer can be a sarcoma. The cancer can be a leukemia. The cancer can be a solid tumor.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

Carcinomas that can be amenable to therapy by a method disclosed herein include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma.

Sarcomas that can be amenable to therapy by a method disclosed herein include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

The modified cells (e.g., immune cells, T cells, or NK cells) described herein can be included in a composition for immunotherapy. The composition can include a pharmaceutical composition and further include a pharmaceutically acceptable carrier. A therapeutically effective amount of the pharmaceutical composition comprising the modified cells can be administered.

The modified cells can be immediately used in the above therapeutic, experimental or commercial applications or the cells can be cryopreserved for use at a later date. The pharmaceutical compositions can be included in a container, pack, or dispenser together with instructions for administration.

The modified cells disclosed herein can be formulated in unit dosage forms suitable for single administration of precise dosages. In some cases, the unit dosage forms comprise additional lymphocytes. In unit dosage form, the formulation is divided into unit doses containing appropriate quantities of one or more compounds. The unit dosage can be in the form of a package containing discrete quantities of the formulation. Non-limiting examples are packaged tablets or capsules, and powders in vials or ampoules. Aqueous suspension compositions can be packaged in single-dose non-reclosable containers. Multiple-dose reclosable containers can be used, for example, in combination with a preservative or without a preservative. In some examples, the pharmaceutical composition does not comprise a preservative. Formulations for parenteral injection can be presented in unit dosage form, for example, in ampoules, or in multi-dose containers with a preservative.

### EXAMPLES

The disclosure is further described in the following examples, which do not limit the scope of the disclosure described in the claims.

### Example 1. Plasmid Construction, Virus Preparation, and Titer Evaluation

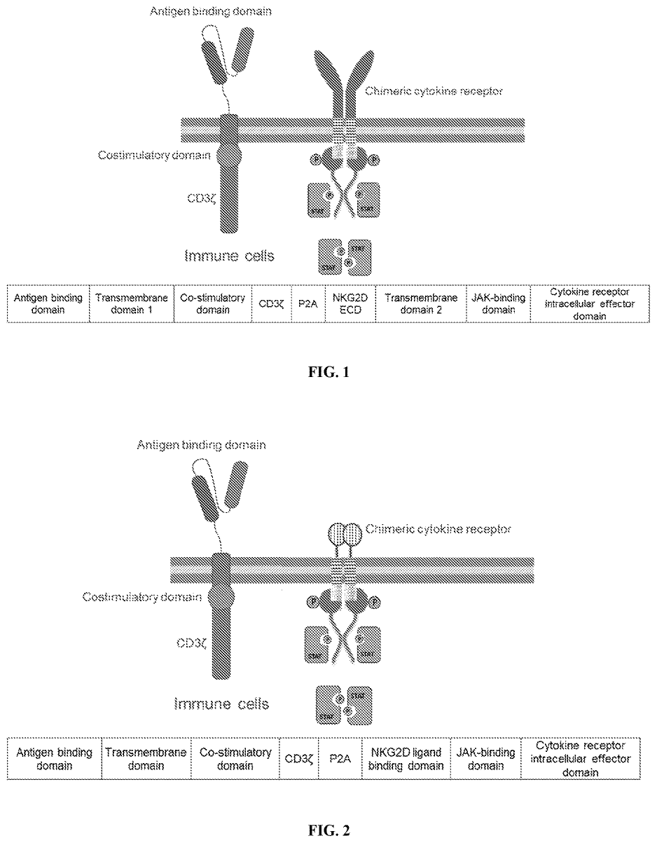

FIGS. 1-2 show schematics of designs of CAR T cells armored with different chimeric receptors.

A construct encoding anti-GPC3 benchmark CAR (“BM CAR”) with 4-1BB co-stimulatory domain (SEQ ID NO: 4) was prepared for comparative analysis.

To generate viral particles comprising nucleic acids encoding any of the systems disclosed herein, lentivirus packaging plasmid mixture including pMDLg/pRRE (Addgene #11251), pRSV-Rev (Addgene #11253), and pMD2.G (Addgene #11259) were pre-mixed with a PLVX-EF1A (including target system) vector at a pre-optimized ratio with polyetherimide (PEI), mixed properly, and incubated at room temperature for 5 minutes. The transfection mixture was added dropwise to 293-T cells and mixed gently. Transfected 293-T cells were incubated overnight at 37° C. with 5% CO₂. Twenty-four hours post-transfection, supernatants were collected and centrifuged at 4° C., 500 g for 10 min to remove any cellular debris. Centrifuged supernatants were filtered through a 0.45 μm PES filter to concentrate the viral supernatants post ultracentrifugation. After centrifugation, the supernatants were carefully discarded, and the virus pellets were rinsed with pre-chilled DPBS (Dulbecco's phosphate-buffered saline). The concentration of virus was measured. Virus was aliquoted and stored at −80° C. Viral titers were determined by functional transduction on a T cell line.

The lentiviral vector was modified using pLVX-Puro (Clontech #632164) by replacing the original promoter with human elongation factor 1α promoter (hEF1α) and by removing the puromycin resistance gene with EcoRI and BamHI by GenScript. PLVX-EF1A was further subjected to the lentivirus packaging procedure as described above.

### Example 2: Collection and Transduction of γδ T Lymphocytes

γδ T cells were prepared by addition of 5 μM Zoledronate and 1000 IU/mL IL-2 to PBMCs and cultured for 9 days with periodical change of media supplemented with 1000 IU/mL IL-2. Alternatively, γδ T cells were isolated from PBMC or umbilical cord blood (UCB) and then stimulated by anti-γδ TCR antibody and anti-CD3 (OKT3) followed by co-incubation of K562-based artificial antigen-presenting cells (aAPCs) at 1:2 ratio for at least 10 days.

PBMCs were isolated by density centrifugation (lymphoprep) from leukapheresis material and cryopreserved. PBMCs were resuscitated and activated with zoledronic acid (5 μM) in cell culture media AIM-V supplemented with IL-2 (1000 IU/ml) and 5% human AB serum and kept in a humidified chamber (37° C., 5% CO₂). 48 hours post-activation, cells were transduced with lentiviral vectors encoding the system of Example 1 at an MOI of 5 with 5 μg/ml polybrene. Such transduction procedure was repeated the next day followed by replenishment of fresh media containing IL-2 (1000 IU/ml) the second day after the transduction. Cells were cultured in AIM-V supplemented with IL-2 (1000 IU/ml) in a humidified chamber with periodical change of media as determined by the pH of the culture media for further expansion. Cells were harvested 10 days post-transduction and the total number, purity and transduction efficiency were determined. Cells were further enriched with a negative TCRγ/δ⁺ T cell isolation kit (Miltenyi Biotec) before future applications or cryopreserved.

### Example 3: Long-Term Cytotoxicity Assay of Anti-GPC3 BM-CAR γδ T Cells

To evaluate the long-term killing efficacy of CAR γδ T cells, we performed long-term co-culture assays, which mimic the dynamic killing process in vivo. Transduced or non-transduced T cells (1×10⁵/well) were co-cultured with tumor cell lines (Huh7 cells, 1×10⁵/well) at an E:T ratio of 1:1 in 24-well plates, in the absence of exogenous cytokines (IL-2). Part of the cells were harvested and stained for CD3 after 2 to 3 days co-culture. For serial co-culture assays, the remaining T cells were then re-challenged with fresh Huh7 cells at the same E:T ratio. Co-cultures were carried on until tumor cells outgrew. The T cell proliferation rate at each time point was calculated by dividing the number of T cells at the time point by the number of T cells at a previous time point.

### Engineering of NKG2D Chimeric Cytokine Receptor Using Different Transmembrane Domain

To test other transmembrane domains' role in NKG2D chimeric cytokine receptor, we replaced the transmembrane domain of TPOR with CD28 or CD8, which are usually used in CAR T cells, and evaluated the long-term killing efficacy of CAR γδ T cells armored with these designs.

BM-NKG2D-TPOR TM-TPOR (BOX1-BOX2)-IL-15Rβ-IL-21R (SEQ ID NO: 22), also named BM-15-21: a CAR backbone sequence encoding a CAR backbone polypeptide comprising from the N-terminus to the C-terminus: a signal peptide (SEQ ID NO: 1), a GPC3 BM-CAR (SEQ ID NO: 4), via a P2A cleavage site (SEQ ID NO: 5), and is further connected to a NKG2D chimeric cytokine receptor molecule comprising from the N-terminus to the C-terminus: a signal peptide (SEQ ID NO: 1), a NKG2D ECD (SEQ ID NO: 6), a TPOR transmembrane (TM) (SEQ ID NO: 9) and TPOR BOX1-BOX2 (SEQ ID NO: 7), a IL-15Rβ ICD (SEQ ID NO: 10) and a IL-21R ICD (SEQ ID NO: 20).

BM-NKG2D-CD8 TM-TPOR (BOX1-BOX2)-IL-15Rβ-IL-21R (SEQ ID NO: 23): a CAR backbone sequence encoding a CAR backbone polypeptide comprising from the N-terminus to the C-terminus: a signal peptide (SEQ ID NO: 1), a GPC3 BM-CAR (SEQ ID NO: 4), via a P2A cleavage site (SEQ ID NO: 5), and is further connected to a NKG2D chimeric cytokine receptor molecule comprising from the N-terminus to the C-terminus: a signal peptide (SEQ ID NO: 1), a NKG2D ECD (SEQ ID NO: 6), a CD8 hinge (SEQ ID NO: 86), a CD8 TM (SEQ ID NO: 2), a TPOR BOX1-BOX2 (SEQ ID NO: 7), a IL-15Rβ ICD (SEQ ID NO: 10) and a IL-21R ICD (SEQ ID NO: 20).

BM-NKG2D-CD28 TM-TPOR (BOX1-BOX2)-IL-15Rβ-IL-21R (SEQ ID NO: 24): a CAR backbone sequence encoding a CAR backbone polypeptide comprising from the N-terminus to the C-terminus: a signal peptide (SEQ ID NO: 1), a GPC3 BM-CAR (SEQ ID NO: 4), via a P2A cleavage site (SEQ ID NO: 5), and is further connected to a NKG2D chimeric cytokine receptor molecule comprising from the N-terminus to the C-terminus: a signal peptide (SEQ ID NO: 1), a NKG2D ECD (SEQ ID NO: 6), a CD28 TM (SEQ ID NO: 3), a TPOR BOX1-BOX2 (SEQ ID NO: 7), a IL-15Rβ ICD (SEQ ID NO: 10) and a IL-21R ICD (SEQ ID NO: 20).

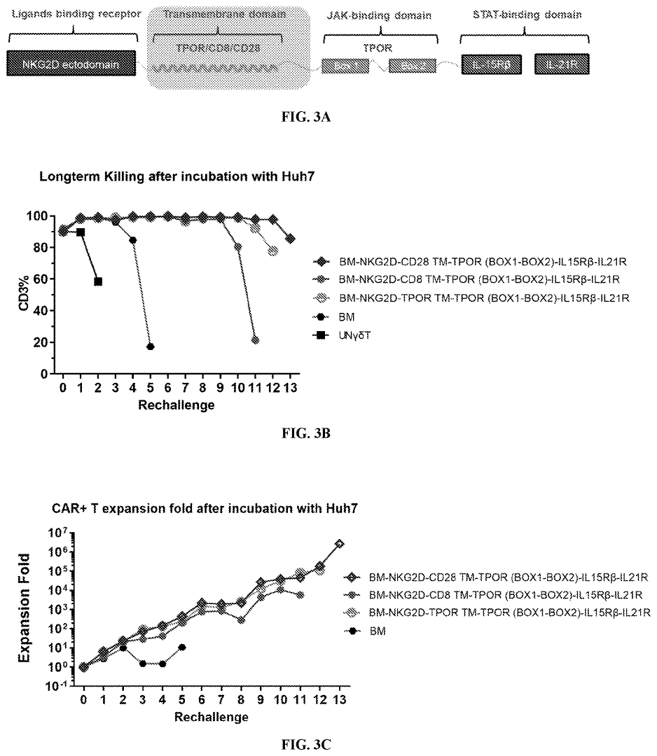

FIG. 3A shows the schematic of GPC3 CAR γδ T cells armored with NKG2D chimeric cytokine receptor using different transmembrane domain. FIG. 3B and FIG. 3C show the long-term killing efficacy and proliferation of GPC3 CAR γδ T cells against GPC3⁺ target cells Huh7, respectively. GPC3 CAR γδ T cells armored with NKG2D chimeric cytokine receptor using CD8 transmembrane domain enhanced CAR γδ T cells cytotoxicity and proliferation compared to naked CAR γδ T cells, but was inferior to that using TPOR transmembrane domain, which might be due to armor interfering with CAR since both used CD8 transmembrane domain. GPC3 CAR γδ T cells armored with NKG2D chimeric cytokine receptor using CD28 transmembrane domain was superior to that using TPOR transmembrane domain. Therefore, using CD8, CD28, TPOR or other transmembrane domains all could activate NKG2D chimeric cytokine receptor, and were chosen by context.

Engineering of NKG2D Chimeric Cytokine Receptor with or without TPOR BOX2

JAK-binding domains usually contain both a BOX1 and BOX2 motif, and the BOX2 region of the TPOR is important for maximal TPOR mitogenic activity and maximal JAK2 activation (see, e.g., Tong W. et al. The membrane-proximal region of the thrombopoietin receptor confers its high surface expression by JAK2-dependent and -independent mechanisms. J Biol Chem. 2006 Dec. 15; 281 (50): 38930-40).

To evaluate the role of TPOR BOX2, we generated NKG2D chimeric cytokine receptor with or without TPOR BOX2 and tested the long-term killing efficacy of CAR γδ T cells armored with these designs.

BM-NKG2D-TPOR TM-TPOR (BOX1)-IL-15Rβ-IL-21R (SEQ ID NO: 25): a CAR backbone sequence encoding a CAR backbone polypeptide comprising from the N-terminus to the C-terminus: a signal peptide (SEQ ID NO: 1), a GPC3 BM-CAR (SEQ ID NO: 4), via a P2A cleavage site (SEQ ID NO: 5), and is further connected to a NKG2D chimeric cytokine receptor molecule comprising from the N-terminus to the C-terminus: a signal peptide (SEQ ID NO: 1), a NKG2D ECD (SEQ ID NO: 6), a TPOR TM (SEQ ID NO: 9) and TPOR-BOX1 (SEQ ID NO: 8), a IL-15Rβ ICD (SEQ ID NO: 10) and a IL-21R ICD (SEQ ID NO: 20).

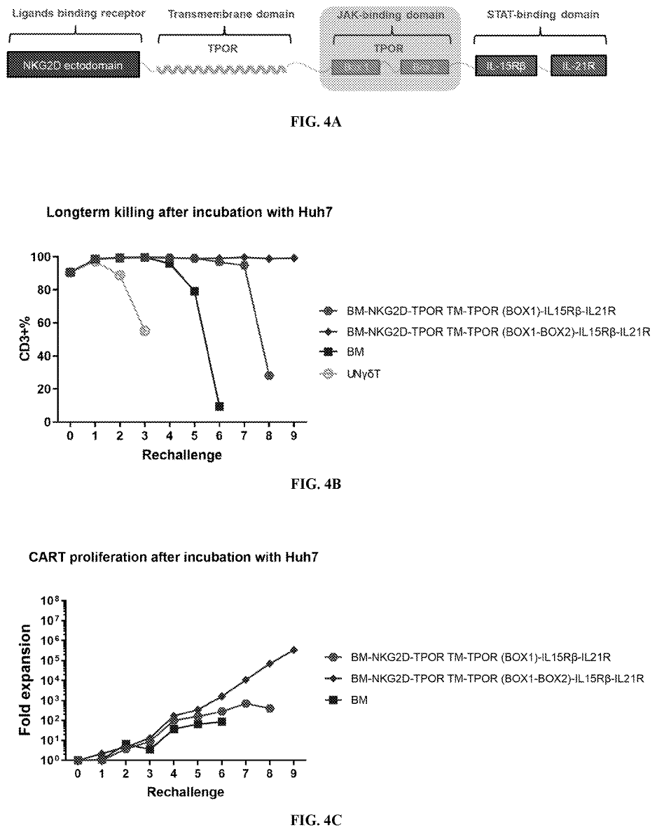

FIG. 4A shows the schematic of GPC3 CAR γδ T cells armored with NKG2D chimeric cytokine receptor with or without TPOR BOX2. FIG. 4B and FIG. 4C show the long-term killing efficacy and proliferation of GPC3 CAR γδ T cells against Huh7 cells, respectively. GPC3 CAR γδ T cells armored with NKG2D chimeric cytokine receptor deletion TPOR BOX2 motif enhanced CAR γδ T cells cytotoxicity and proliferation compared to naked CAR γδ T cells, but was inferior to that with TPOR BOX2 motif in vitro, which reveals TPOR BOX2 plays part role in NKG2D chimeric cytokine receptor.

### Engineering of NKG2D Chimeric Cytokine Receptor Using IL-15Rβ JAK-Binding Domain

IL-15Rβ contains its own JAK-binding domain (see, e.g., Usacheva A. et al. Contribution of the Box 1 and Box 2 motifs of cytokine receptors to Jak1 association and activation. J Biol Chem. 2002 Dec. 13; 277 (50): 48220-6). To evaluate the different roles of TPOR JAK-binding domain and IL-15Rβ JAK-binding domain, we generated NKG2D chimeric cytokine receptor using TPOR or IL-15Rβ JAK-binding domain and tested the long-term killing efficacy of CAR γδ T cells armored with these designs.

BM-NKG2D-TPOR TM-IL-15Rβ (BOX1-BOX2)-IL-15Rβ-IL-21R (SEQ ID NO: 26): a CAR backbone sequence encoding a CAR backbone polypeptide comprising from the N-terminus to the C-terminus: a signal peptide (SEQ ID NO: 1), a GPC3 BM-CAR (SEQ ID NO: 4), via a P2A cleavage site (SEQ ID NO: 5), and is further connected to a NKG2D chimeric cytokine receptor molecule comprising from the N-terminus to the C-terminus: a signal peptide (SEQ ID NO: 1), a NKG2D ECD (SEQ ID NO: 6), a TPOR TM (SEQ ID NO: 9) and IL-15Rβ BOX1-BOX2 (SEQ ID NO: 19), a IL-15Rβ ICD (SEQ ID NO: 10) and a IL-21R ICD (SEQ ID NO: 20).

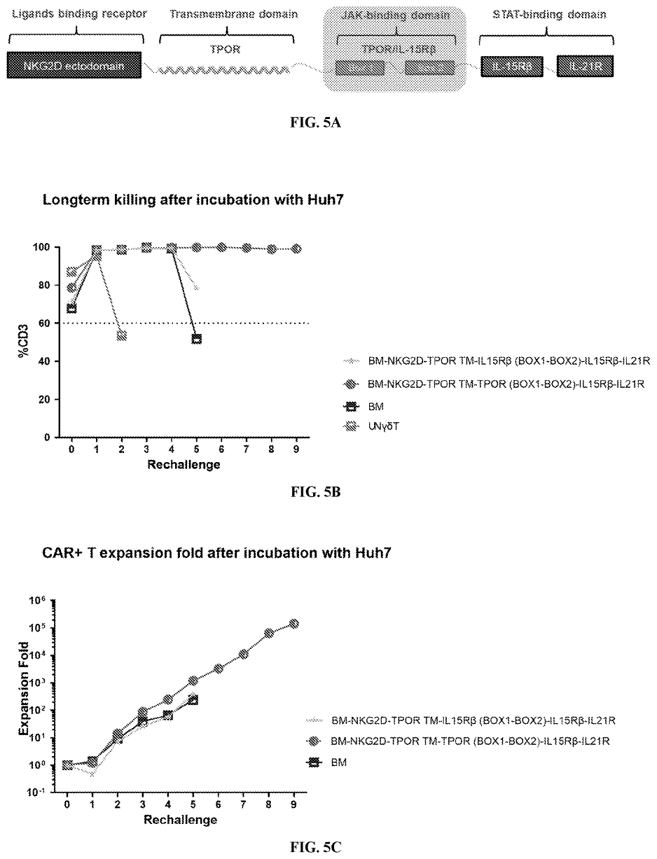

FIG. 5A shows the schematic of GPC3 CAR γδ T cells armored with NKG2D chimeric cytokine receptor using TPOR or IL-15Rβ JAK-binding domain. FIG. 5B and FIG. 5C show the long-term killing efficacy and proliferation of GPC3 CAR γδ T cells against Huh7 cells, respectively. GPC3 CAR γδ T cells armored with NKG2D chimeric cytokine receptor using IL-15Rβ JAK-binding domain didn't enhanced CAR γδ T cells cytotoxicity and proliferation compared to naked CAR γδ T cells, and was far inferior to that using TPOR JAK-binding domain in vitro, which reveals the important role of TPOR JAK-binding domain in NKG2D chimeric cytokine receptor and TPOR BOX1 is indispensable.

### Engineering of NKG2D Chimeric Cytokine Receptor Using CD28 Transmembrane Domain and TPOR BOX1

According to the above results, we further analyzed how the combination of CD28 transmembrane domain and deletion TPOR BOX2 would activate NKG2D chimeric cytokine receptor, and generated NKG2D chimeric cytokine receptor using CD28 transmembrane domain and TPOR BOX1, and tested the long-term killing efficacy of CAR γδ T cells armored with these designs.

BM-NKG2D-CD28 TM-TPOR (BOX1)-IL-15Rβ-IL-21R (SEQ ID NO: 27) is a CAR backbone sequence encoding a CAR backbone polypeptide comprising from the N-terminus to the C-terminus: a signal peptide (SEQ ID NO: 1), a GPC3 BM-CAR (SEQ ID NO: 4), via a P2A cleavage site (SEQ ID NO: 5), and is further connected to a NKG2D chimeric cytokine receptor molecule comprising from the N-terminus to the C-terminus: a signal peptide (SEQ ID NO: 1), a NKG2D ECD (SEQ ID NO: 6), a CD28 TM (SEQ ID NO: 3), a TPOR-BOX1 (SEQ ID NO: 8), a IL-15Rβ ICD (SEQ ID NO: 10) and a IL-21R ICD (SEQ ID NO: 20).

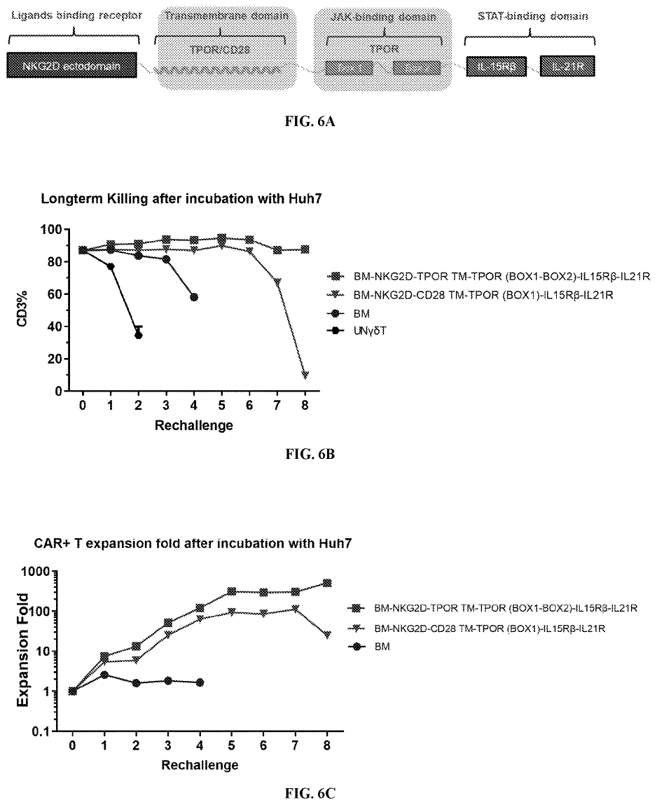

FIG. 6A shows the schematic of GPC3 CAR γδ T cells armored with NKG2D chimeric cytokine receptor using CD28 transmembrane domain and TPOR BOX1. FIG. 6B and FIG. 6C show the long-term killing efficacy and proliferation of GPC3 CAR γδ T cells against Huh7 cells, respectively. GPC3 CAR γδ T cells armored with NKG2D chimeric cytokine receptor using CD28 transmembrane domain and TPOR BOX1 enhanced CAR γδ T cells cytotoxicity and proliferation compared to naked CAR γδ T cells, and was slightly inferior to that using TPOR transmembrane domain and TPOR BOX1-BOX2 in vitro, which reveals that combination of CD28 transmembrane domain and TPOR BOX1 would effectively activate NKG2D chimeric cytokine receptor.

Engineering of NKG2D Chimeric Cytokine Receptor with Two Functionally Synergistic STAT-Binding Domains

As cytokines often have synergistic effects on CAR T cells, we generated NKG2D chimeric cytokine receptor with two STAT-binding domains in tandem, and tested the long-term killing efficacy of CAR γδ T cells armored with these designs.

BM-NKG2D-TPOR TM-TPOR (BOX1-BOX2)-IL-21R-IL-15Rβ (SEQ ID NO: 28): a CAR backbone sequence encoding a CAR backbone polypeptide comprising from the N-terminus to the C-terminus: a signal peptide (SEQ ID NO: 1), a GPC3 BM-CAR (SEQ ID NO: 4), via a P2A cleavage site (SEQ ID NO: 5), and is further connected to a NKG2D chimeric cytokine receptor molecule comprising from the N-terminus to the C-terminus: a signal peptide (SEQ ID NO: 1), a NKG2D ECD (SEQ ID NO: 6), a TPOR TM (SEQ ID NO: 9) and TPOR BOX1-BOX2 (SEQ ID NO: 7), a IL-21R ICD (SEQ ID NO: 20) and a IL-15Rβ ICD (SEQ ID NO: 10).

BM-15-21mDO (Y519) (SEQ ID NO: 29): a CAR backbone sequence encoding a CAR backbone polypeptide comprising from the N-terminus to the C-terminus: a signal peptide (SEQ ID NO: 1), a GPC3 BM-CAR (SEQ ID NO: 4), via a P2A cleavage site (SEQ ID NO: 5), and is further connected to a NKG2D chimeric cytokine receptor molecule comprising from the N-terminus to the C-terminus: a signal peptide (SEQ ID NO: 1), a NKG2D ECD (SEQ ID NO: 6), a TPOR TM (SEQ ID NO: 9) and TPOR BOX1-BOX2 (SEQ ID NO: 7), a IL-15Rβ ICD (SEQ ID NO: 10) and a IL-21R ICD with Y519F mutation (SEQ ID NO: 21).

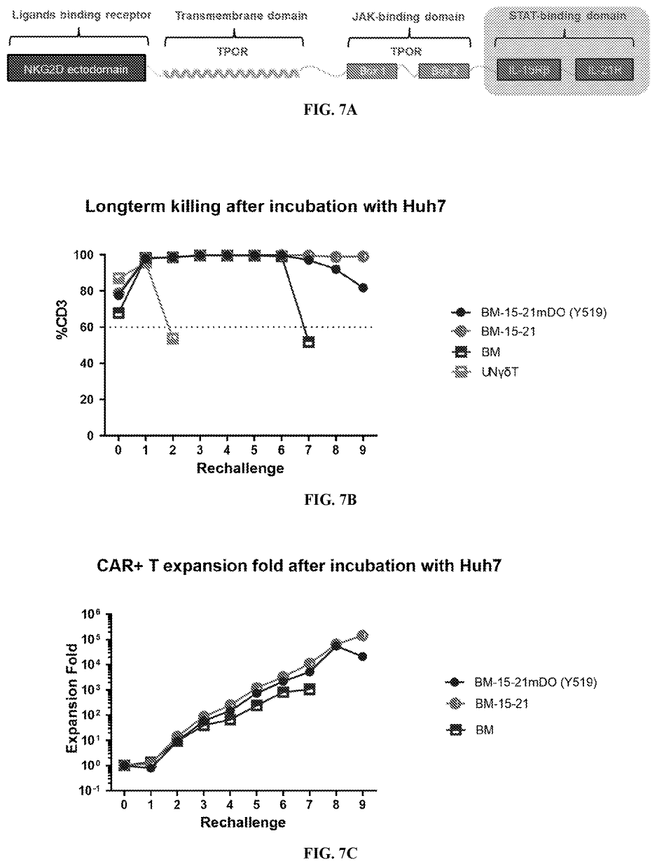

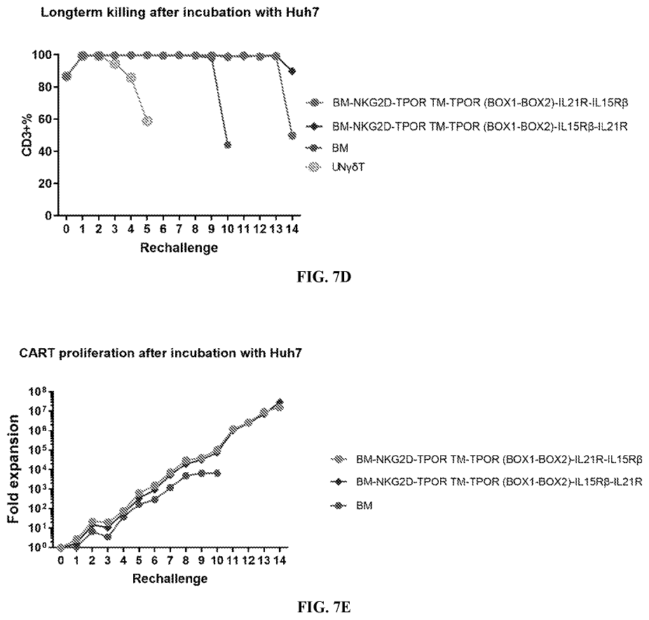

FIG. 7A shows the schematic of GPC3 CAR γδ T cells armored with NKG2D chimeric cytokine receptor with two functionally synergistic STAT-binding domains. FIG. 7B and FIG. 7C show the long-term killing efficacy and proliferation of GPC3 CAR γδ T cells against Huh7 cells, respectively. GPC3 CAR γδ T cells armored with NKG2D chimeric cytokine receptor using two STAT-binding domains enhanced CAR γδ T cells cytotoxicity and proliferation compared to naked CAR γδ T cells, but the IL-21R functional site mutation (Y519F) was inferior to that natural IL-21R in vitro, which reveals the synergistic effects of IL-15Rβ and IL-21R on activating NKG2D chimeric cytokine receptor.

As the proximity of co-stimulatory domains to the cell membrane influence their function, whether individual STAT-binding domains to the cell membrane influence NKG2D chimeric cytokine receptor function. We generated NKG2D chimeric cytokine receptor with IL-15Rβ and IL-21R STAT-binding domains, each STAT-binding domain was proximal or distal to the cell membrane, and tested the long-term killing efficacy of CAR γδ T cells armored with these designs.

FIG. 7D and FIG. 7E show the long-term killing efficacy and proliferation of GPC3 CAR γδ T cells against Huh7 cells, respectively. GPC3 CAR γδ T cells armored with NKG2D chimeric cytokine receptor using two STAT-binding domains enhanced CAR γδ T cells cytotoxicity and proliferation compared to naked CAR γδ T cells, but whether IL-15Rβ or IL-21R STAT-binding domains was proximal to the cell membrane didn't affect the activation of NKG2D chimeric cytokine receptor.

### Engineering of NKG2D Chimeric Cytokine Receptor Identifying the Functional Sites of IL-15Rβ STAT-Binding Domain

BM-15mDO (Y364)-21 (SEQ ID NO: 30): a CAR backbone sequence encoding a CAR backbone polypeptide comprising from the N-terminus to the C-terminus: a signal peptide (SEQ ID NO: 1), a GPC3 BM-CAR (SEQ ID NO: 4), via a P2A cleavage site (SEQ ID NO: 5), and is further connected to a NKG2D chimeric cytokine receptor molecule comprising from the N-terminus to the C-terminus: a signal peptide (SEQ ID NO: 1), a NKG2D ECD (SEQ ID NO: 6), a TPOR TM (SEQ ID NO: 9) and TPOR BOX1-BOX2 (SEQ ID NO: 7), a IL-15Rβ ICD with Y364F mutation (SEQ ID NO: 11) and a IL-21R ICD (SEQ ID NO: 20).

BM-15mDO (Y364, 381, 384, 387)-21 (SEQ ID NO: 31) is a CAR backbone sequence encoding a CAR backbone polypeptide comprising from the N-terminus to the C-terminus: a signal peptide (SEQ ID NO: 1), a GPC3 BM-CAR (SEQ ID NO: 4), via a P2A cleavage site (SEQ ID NO: 5), and is further connected to a NKG2D chimeric cytokine receptor molecule comprising from the N-terminus to the C-terminus: a signal peptide (SEQ ID NO: 1), a NKG2D ECD (SEQ ID NO: 6), a TPOR TM (SEQ ID NO: 9) and TPOR BOX1-BOX2 (SEQ ID NO: 7), a IL-15Rβ ICD with Y364F, Y381F, Y384F, Y387F mutation (SEQ ID NO: 12) and a IL-21R ICD (SEQ ID NO: 20).

BM-15mDO (Y418)-21 (SEQ ID NO: 32) is a CAR backbone sequence encoding a CAR backbone polypeptide comprising from the N-terminus to the C-terminus: a signal peptide (SEQ ID NO: 1), a GPC3 BM-CAR (SEQ ID NO: 4), via a P2A cleavage site (SEQ ID NO: 5), and is further connected to a NKG2D chimeric cytokine receptor molecule comprising from the N-terminus to the C-terminus: a signal peptide (SEQ ID NO: 1), a NKG2D ECD (SEQ ID NO: 6), a TPOR TM (SEQ ID NO: 9) and TPOR BOX1-BOX2 (SEQ ID NO: 7), a IL-15Rβ ICD with Y418F mutation (SEQ ID NO: 13) and a IL-21R ICD (SEQ ID NO: 20).

BM-15mDO (Y536)-21 (SEQ ID NO: 33) is a CAR backbone sequence encoding a CAR backbone polypeptide comprising from the N-terminus to the C-terminus: a signal peptide (SEQ ID NO: 1), a GPC3 BM-CAR (SEQ ID NO: 4), via a P2A cleavage site (SEQ ID NO: 5), and is further connected to a NKG2D chimeric cytokine receptor molecule comprising from the N-terminus to the C-terminus: a signal peptide (SEQ ID NO: 1), a NKG2D ECD (SEQ ID NO: 6), a TPOR TM (SEQ ID NO: 9) and TPOR BOX1-BOX2 (SEQ ID NO: 7), a IL-15Rβ ICD with Y536F mutation (SEQ ID NO: 14) and a IL-21R ICD (SEQ ID NO: 20).

BM-15mDO (Y418, 536)-21 (SEQ ID NO: 34) is a CAR backbone sequence encoding a CAR backbone polypeptide comprising from the N-terminus to the C-terminus: a signal peptide (SEQ ID NO: 1), a GPC3 BM-CAR (SEQ ID NO: 4), via a P2A cleavage site (SEQ ID NO: 5), and is further connected to a NKG2D chimeric cytokine receptor molecule comprising from the N-terminus to the C-terminus: a signal peptide (SEQ ID NO: 1), a NKG2D ECD (SEQ ID NO: 6), a TPOR TM (SEQ ID NO: 9) and TPOR BOX1-BOX2 (SEQ ID NO: 7), a IL-15Rβ ICD with Y418F, Y536F mutation (SEQ ID NO: 15) and a IL-21R ICD (SEQ ID NO: 20).

BM-15mDO (Y364, 381, 384, 387, 418, 536)-21 (SEQ ID NO: 35) is a CAR backbone sequence encoding a CAR backbone polypeptide comprising from the N-terminus to the C-terminus: a signal peptide (SEQ ID NO: 1), a GPC3 BM-CAR (SEQ ID NO: 4), via a P2A cleavage site (SEQ ID NO: 5), and is further connected to a NKG2D chimeric cytokine receptor molecule comprising from the N-terminus to the C-terminus: a signal peptide (SEQ ID NO: 1), a NKG2D ECD (SEQ ID NO: 6), a TPOR TM (SEQ ID NO: 9) and TPOR BOX1-BOX2 (SEQ ID NO: 7), a IL-15Rβ ICD with Y364F, Y381F, Y384F, Y387F, Y418F, Y536F mutation (SEQ ID NO: 16) and a IL-21R ICD (SEQ ID NO: 20).

BM-NKG2D-TPOR TM-TPOR (BOX1-BOX2)-IL-15Rβ truncated1-IL-21R (SEQ ID NO: 36) is a CAR backbone sequence encoding a CAR backbone polypeptide comprising from the N-terminus to the C-terminus: a signal peptide (SEQ ID NO: 1), a GPC3 BM-CAR (SEQ ID NO: 4), via a P2A cleavage site (SEQ ID NO: 5), and is further connected to a NKG2D chimeric cytokine receptor molecule comprising from the N-terminus to the C-terminus: a signal peptide (SEQ ID NO: 1), a NKG2D ECD (SEQ ID NO: 6), a TPOR TM (SEQ ID NO: 9) and TPOR BOX1-BOX2 (SEQ ID NO: 7), a IL-15Rβ truncated1 345-431 ICD (SEQ ID NO: 17) and a IL-21R ICD (SEQ ID NO: 20).

BM-NKG2D-TPOR TM-TPOR (BOX1-BOX2)-IL-15Rβ truncated2-IL-21R (SEQ ID NO: 37) is a CAR backbone sequence encoding a CAR backbone polypeptide comprising from the N-terminus to the C-terminus: a signal peptide (SEQ ID NO: 1), a GPC3 BM-CAR (SEQ ID NO: 4), via a P2A cleavage site (SEQ ID NO: 5), and is further connected to a NKG2D chimeric cytokine receptor molecule comprising from the N-terminus to the C-terminus: a signal peptide (SEQ ID NO: 1), a NKG2D ECD (SEQ ID NO: 6), a TPOR TM (SEQ ID NO: 9) and TPOR BOX1-BOX2 (SEQ ID NO: 7), a IL-15Rβ truncated2 345-404 ICD (SEQ ID NO: 18) and a IL-21R ICD (SEQ ID NO: 20).

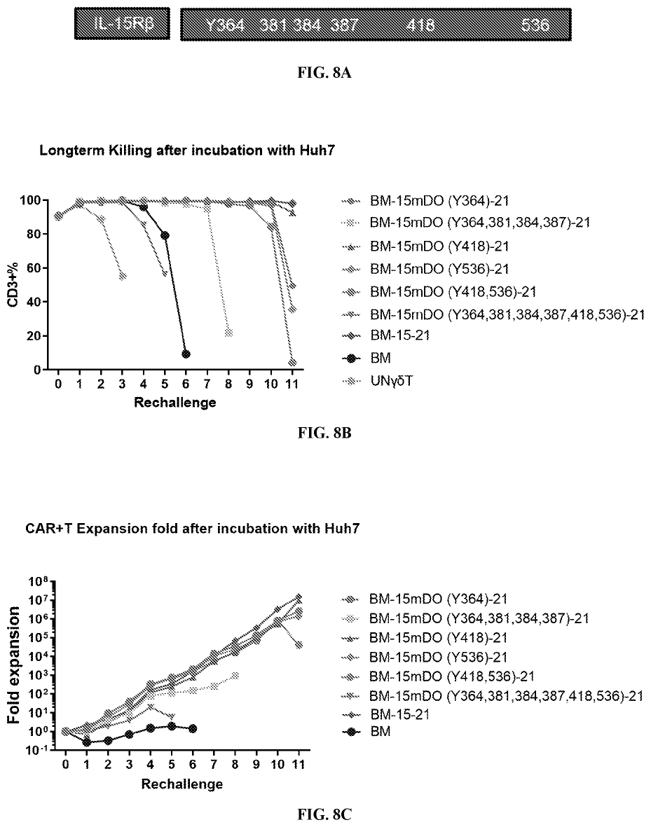

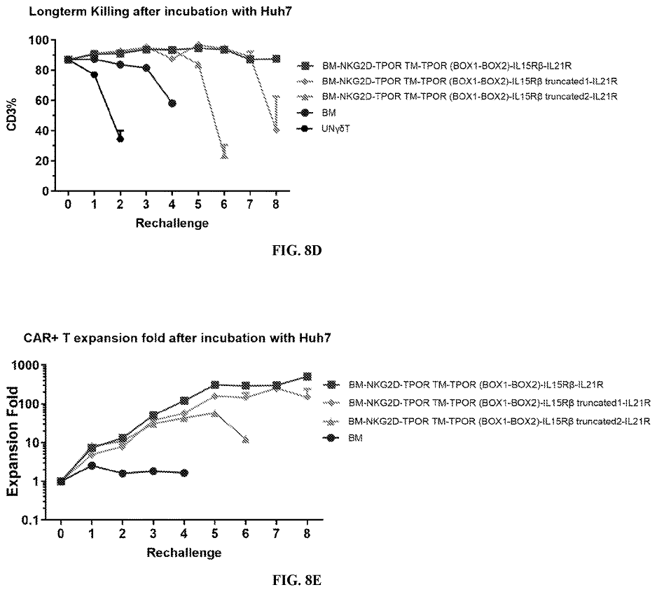

As shown in FIG. 8A, the IL-15Rβ STAT-binding domain we chose contained six phosphorylatable tyrosine residues Y364, Y381, Y384, Y387, Y418 and Y536. To confirm the roles of these tyrosine residues in the activation of NKG2D chimeric cytokine receptor, we mutated these tyrosine residues individually or in combination, and tested the long-term killing efficacy of CAR γδ T cells armored with these designs.

FIG. 8B and FIG. 8C show the long-term killing efficacy and proliferation of GPC3 CAR γδ T cells against Huh7 cells, respectively. Mutation of all six tyrosine residues of IL-15Rβ almost canceled NKG2D chimeric cytokine receptor armor improvement on CAR γδ T cells cytotoxicity, only slightly promoted CAR γδ T cells proliferation, which was contributed by IL-21R. Y418 mutation almost didn't compromise the function of NKG2D chimeric cytokine receptor, while Y364 or Y536 mutation reduced the function of NKG2D chimeric cytokine receptor. What's more, simultaneous mutation of Y418 and Y536 further weakened the function of NKG2D chimeric cytokine receptor, combinatorial mutation of Y364, Y381, Y384, and Y387 greatly alleviated the function of NKG2D chimeric cytokine receptor.

Based on the above results, Y364, Y381, Y384, and Y387 tyrosine residues were important to NKG2D chimeric cytokine receptor, we tried to truncate IL-15Rβ STAT-binding domain containing Y536 or both Y418 and Y536, and tested the long-term killing efficacy of CAR γδ T cells armored with these designs.

FIG. 8D and FIG. 8E show the long-term killing efficacy and proliferation of GPC3 CAR γδ T cells against Huh7 cells, respectively. Truncating IL-15Rβ STAT-binding domain containing Y536 (Truncated1) reduced the function of NKG2D chimeric cytokine receptor, and truncating IL-15Rβ STAT-binding domain containing Y418 and Y536 (Truncated2) further weakened the function of NKG2D chimeric cytokine receptor, which was consistent with mutation results. In summary, full six tyrosine residues or part tyrosine residues can be included depending on the context.

### Example 4: In Vivo Efficacy and Safety Evaluation

Anti-tumor activity of an exemplary anti-GPC3 CAR-T cells was assessed in vivo in a Huh7 xenograft model. Briefly, 3 million (3×10⁶) Huh7 cells were implanted subcutaneously on day 0 in NOD/SCID IL-2Rγ_{C} Null (NSG) mice. Ten days after tumor inoculation, mice were treated with intravenous injection of 1×10⁶ armored CAR-γδ T or mock T cells or phosphate-buffered saline (PBS). Tumor dimensions were measured with calipers twice a week, and tumor volumes were calculated using the formula V=½(length×width²). Mice were euthanized when the mean tumor burden in the control mice reached 2,000 mm³. In addition, the mice plasma was collected for IFN-γ release analysis (Human IFN gamma kit, Cisbio, Cat #62HIFNGPEH). The plasma and a standard were dispensed directly into the assay plate for the cytokine detection utilizing HTRF® reagents. The antibodies labeled with the HTRF donor and acceptor were pre-mixed and added in a single dispensing step.

TABLE 1
Constructs of NKG2D chimeric cytokine receptors
armored CAR-γδ T cells

| Group | Construct |
|---|---|
| BM | BM |
| BM-B6 | BM-NKG2D-TPOR TM-TPOR (BOX1-BOX2)-IL-15Rβ- |
| | IL-21R |
| BM-B6M1 | BM-NKG2D-TPOR TM-TPOR (BOX1-BOX2)-IL-15Rβ |
| | Truncated2-IL-21R |
| BM-B6M2 | BM-NKG2D-CD28 TM-TPOR (BOX1)-IL-15Rβ-IL-21R |
| BM-B6M3 | BM-NKG2D-TPOR TM-TPOR (BOX1-BOX2)-IL-15Rβ |
| | Truncated1-IL-21R |
| BM-B6M4 | BM-NKG2D-CD28 TM-TPOR (BOX1-BOX2)-IL-15Rβ- |
| | IL-21R |

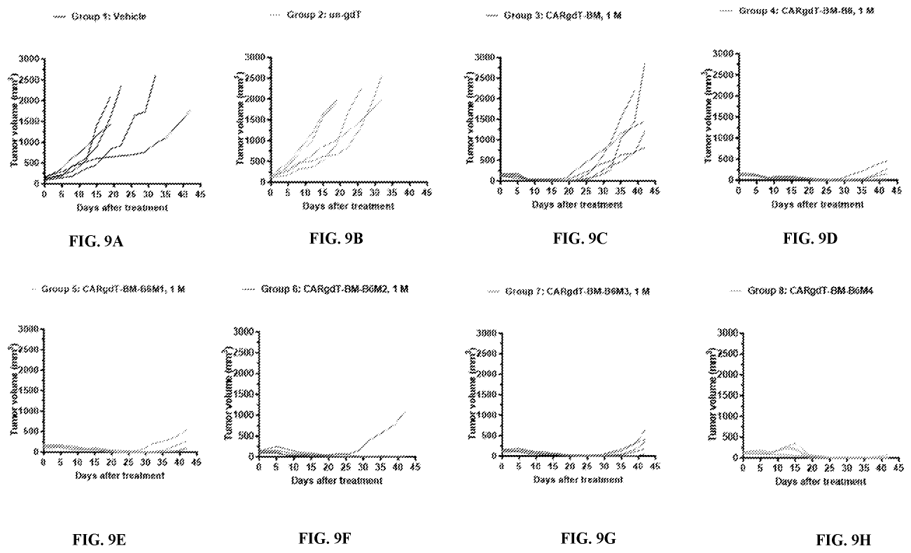

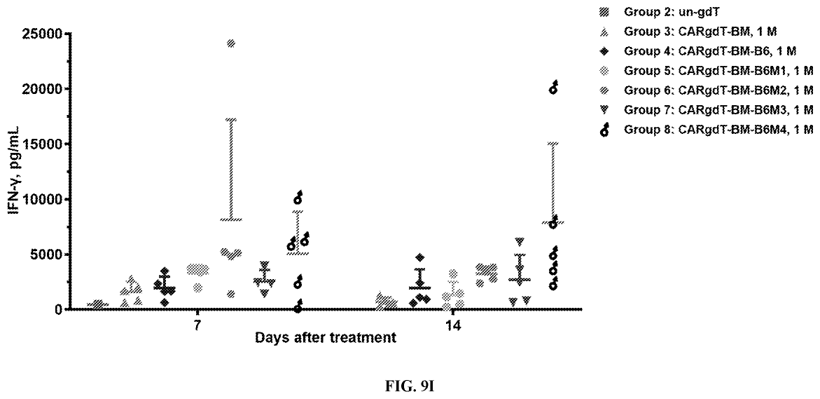

The results of anti-tumor effect of anti-GPC3 CAR γδT cells or anti-GPC3 CAR armored with different NKG2D chimeric cytokine receptors γδT cells in Huh7 xenograft model were shown in FIGS. 9A-9H. Unarmored CAR-γδ T cells, alongside NKG2D chimeric cytokine receptors armored CAR-γδ T cells inhibited tumor growth. Specifically, unarmored CAR-γδT cells-treated mice reached tumor free but quickly relapsed, while NKG2D chimeric cytokine receptors armored CAR-γδ T cells-treated mice reached tumor-free and remained tumor-free or slowly relapsed. Among them, compared with non-deletion tyrosine site of IL-15Rβ (BM-B6), deletion the fragment containing the 536-position (BM-B6M3) or 418- and 536-position (BM-B6M1) tyrosine site of IL-15Rβ were comparable in the degree of tumor suppression and recurrence, which means that 418- and 536-position tyrosine site of IL-15Rβ is dispensable. Compared with NKG2D chimeric cytokine receptor using TPOR transmembrane domain (BM-B6), NKG2D chimeric cytokine receptor using CD28 transmembrane domain (BM-B6M4) had deeper intensity of tumor suppression with less recurrence, which means the CD28 transmembrane domain is more robust. The degree of tumor suppression and recurrence of NKG2D chimeric cytokine receptor deleting TPOR BOX2 (BM-B6M2) was equivalent to that of NKG2D chimeric cytokine receptor containing TPOR BOX2 (BM-B6M4), which means TPOR BOX2 is dispensable.

The results of IFN-γ release of anti-GPC3 CAR γδT cells or anti-GPC3 CAR armored with different NKG2D chimeric cytokine receptors γδT cells in Huh7 xenograft model were shown in FIG. 9I. Armored CAR-γδ T cells induced slightly higher serum levels of IFN-γ than naked CAR-γδ T cells, and NKG2D chimeric cytokine receptor using CD28 transmembrane domain whether or not including TPOR BOX2 (BM-B6M4 and BM-B6M2) armored CAR-γδ T cells released more IFN-γ than NKG2D chimeric cytokine receptor using CD28 transmembrane domain (BM-B6, BM-B6M1 and BM-B6M3) armored CAR-γδ T cells.

### Example 5: Long-Term Cytotoxicity Assay of Other Anti-GPC3 CAR γδ T Cells

### Engineering of NKG2D Chimeric Cytokine Receptor Using B6 or B6M2 Armor Based on Other Anti-GPC3 Binders

In order to rule out that the above conclusions rely on a specific binder, we further evaluated another anti-GPC3 CART using other binders with NKG2D CCR B6 or B6M2 armor, and tested the long-term killing efficacy of CAR γδ T cells armored with these designs. The assay was the same as Example 3.

AZ-CAR were previously disclosed in US20230055143, which recognizes an epitope different from BM (GC33).

AZ-CAR-B6 (SEQ ID NO: 92) is a CAR backbone sequence encoding a CAR backbone polypeptide comprising from the N-terminus to the C-terminus: a signal peptide (SEQ ID NO: 1), a GPC3 AZ-CAR (SEQ ID NO: 91), via a P2A cleavage site (SEQ ID NO: 5), and is further connected to a NKG2D chimeric cytokine receptor molecule comprising from the N-terminus to the C-terminus: a signal peptide (SEQ ID NO: 1), a NKG2D ECD (SEQ ID NO: 6), a TPOR TM (SEQ ID NO: 9), a TPOR-BOX1-BOX2 (SEQ ID NO: 7), a IL-15Rβ ICD (SEQ ID NO: 10) and a IL-21R ICD (SEQ ID NO: 20).

AZ-CAR-B6M2 (SEQ ID NO: 93) is a CAR backbone sequence encoding a CAR backbone polypeptide comprising from the N-terminus to the C-terminus: a signal peptide (SEQ ID NO: 1), a GPC3 AZ-CAR (SEQ ID NO: 91), via a P2A cleavage site (SEQ ID NO: 5), and is further connected to a NKG2D chimeric cytokine receptor molecule comprising from the N-terminus to the C-terminus: a signal peptide (SEQ ID NO: 1), a NKG2D ECD (SEQ ID NO: 6), a CD28 TM (SEQ ID NO: 3), a TPOR-BOX1 (SEQ ID NO: 8), a IL-15Rβ ICD (SEQ ID NO: 10) and a IL-21R ICD (SEQ ID NO: 20).

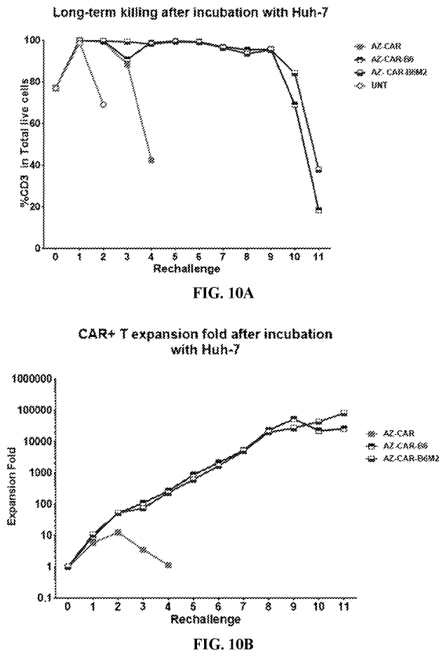

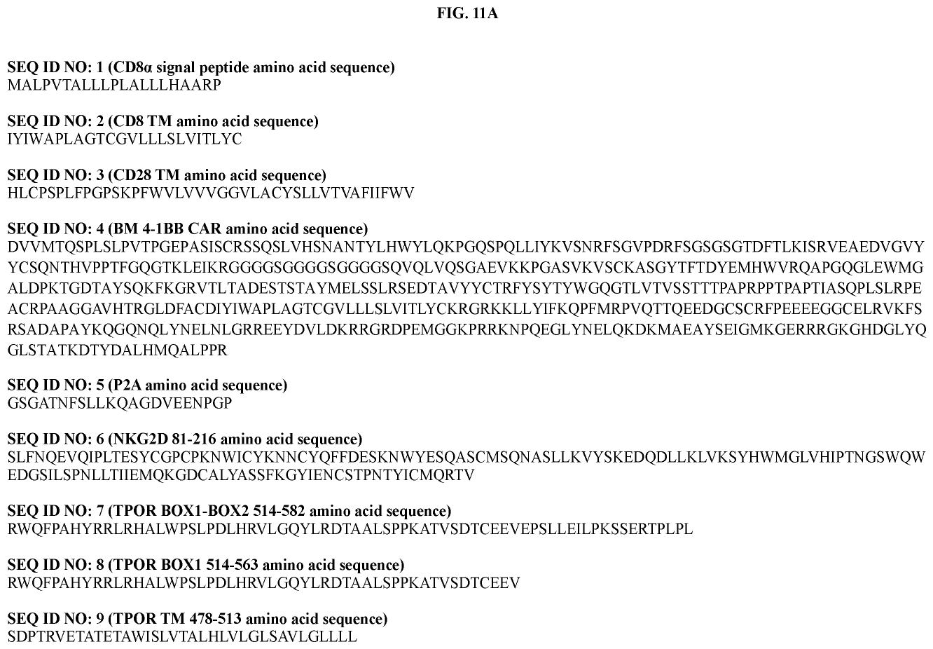

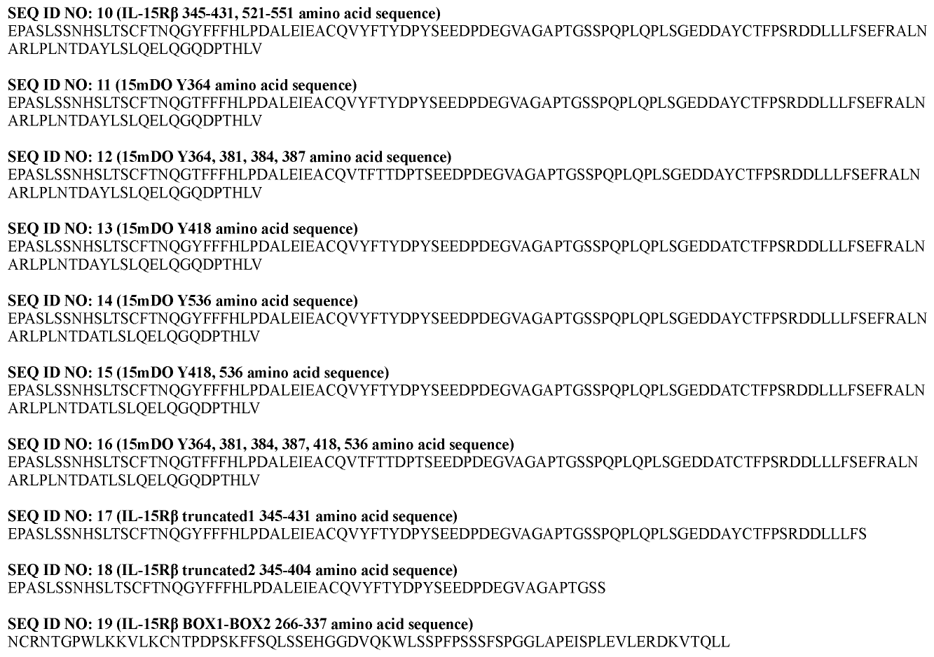

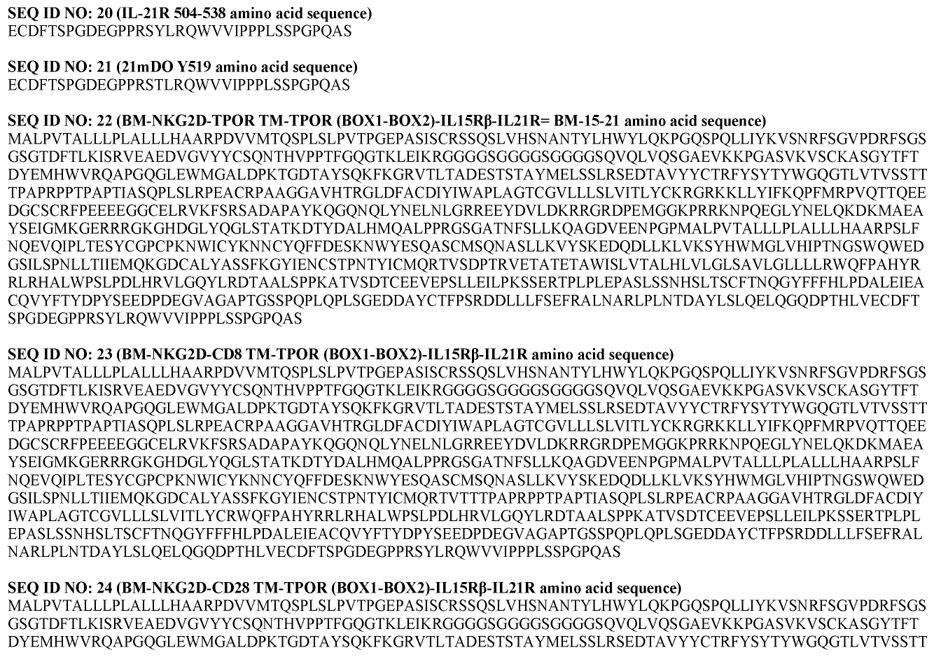

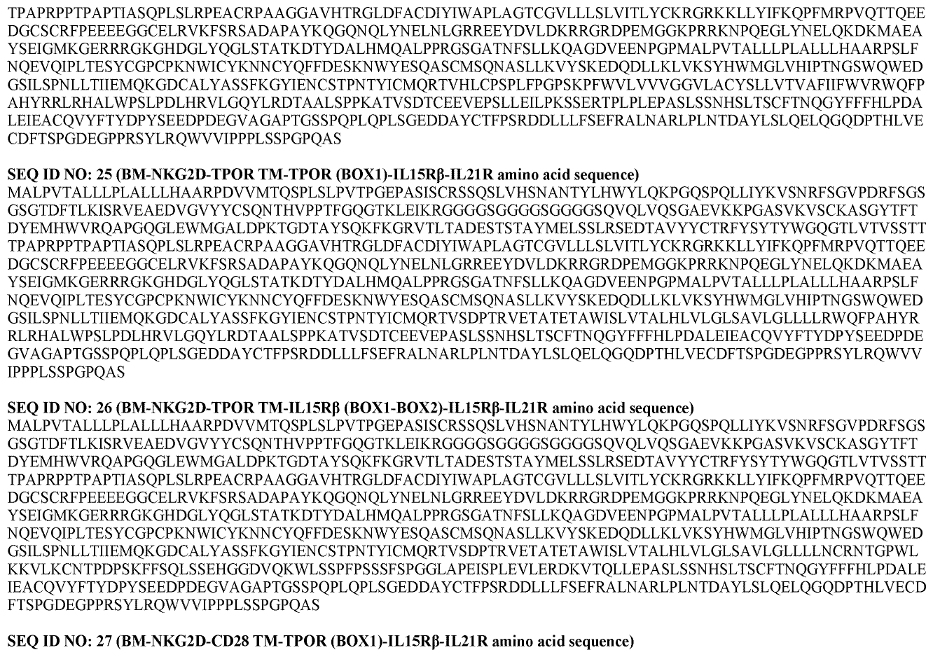

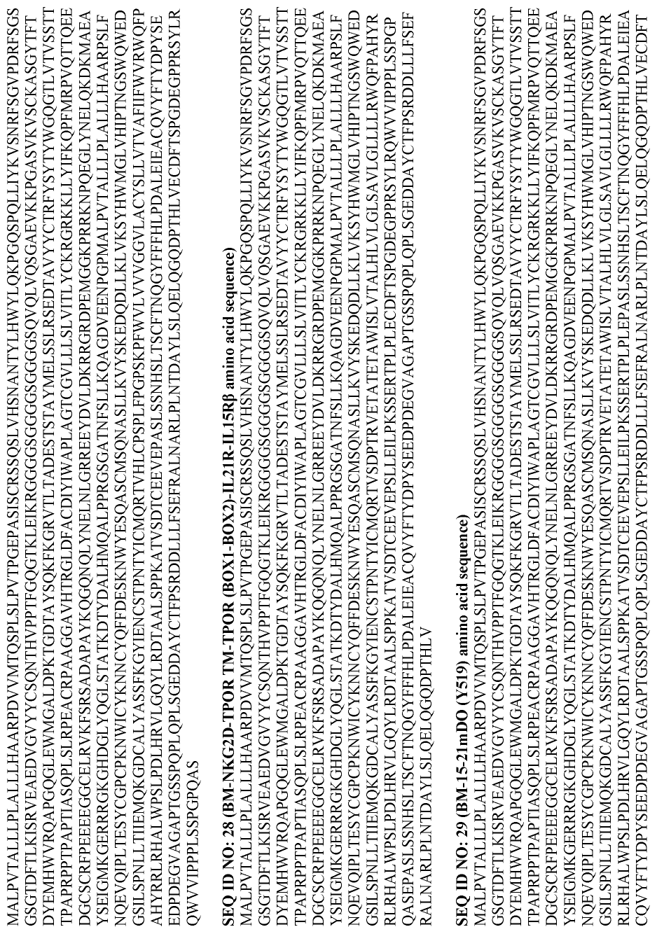

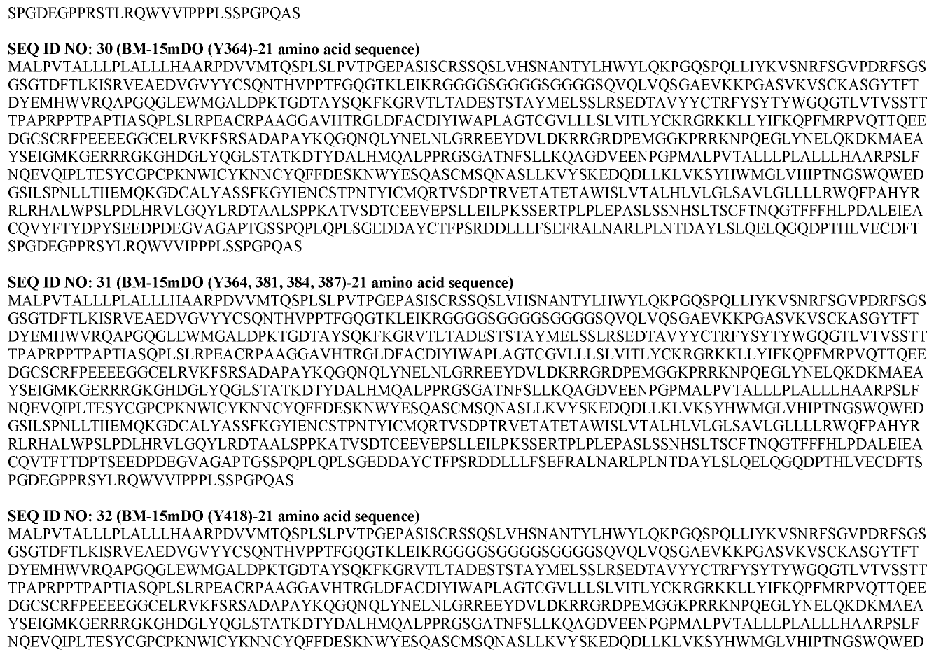

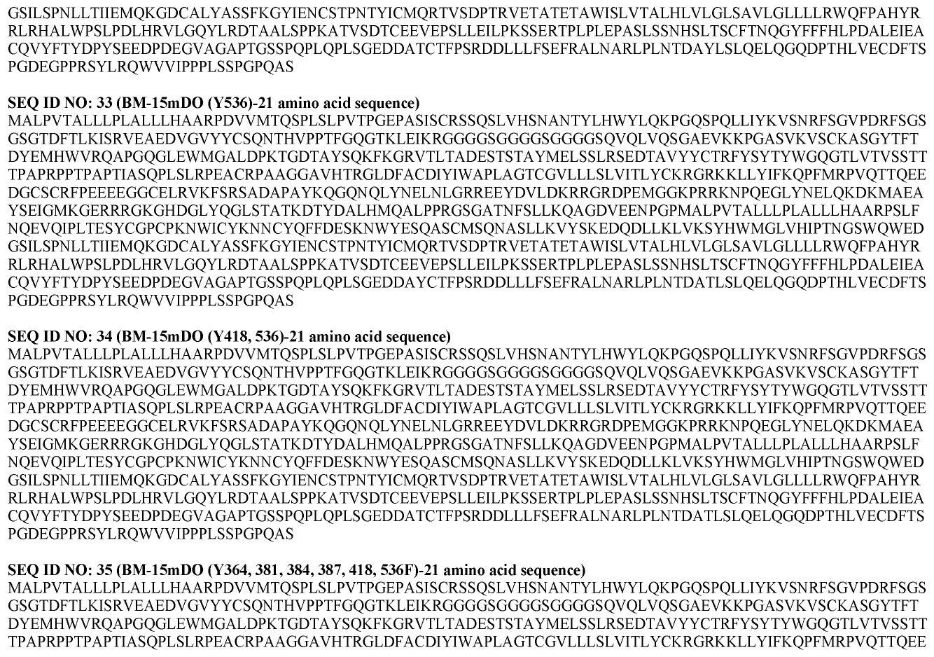

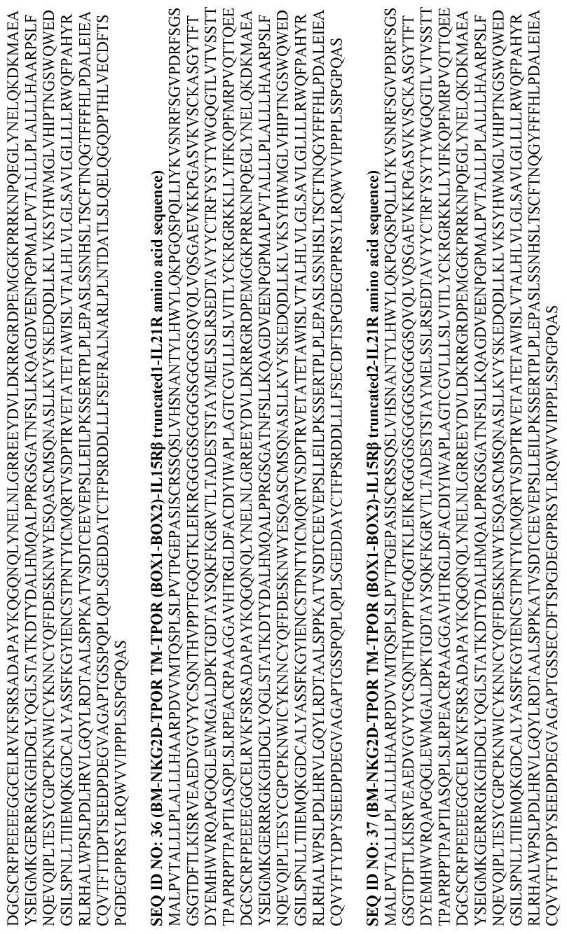

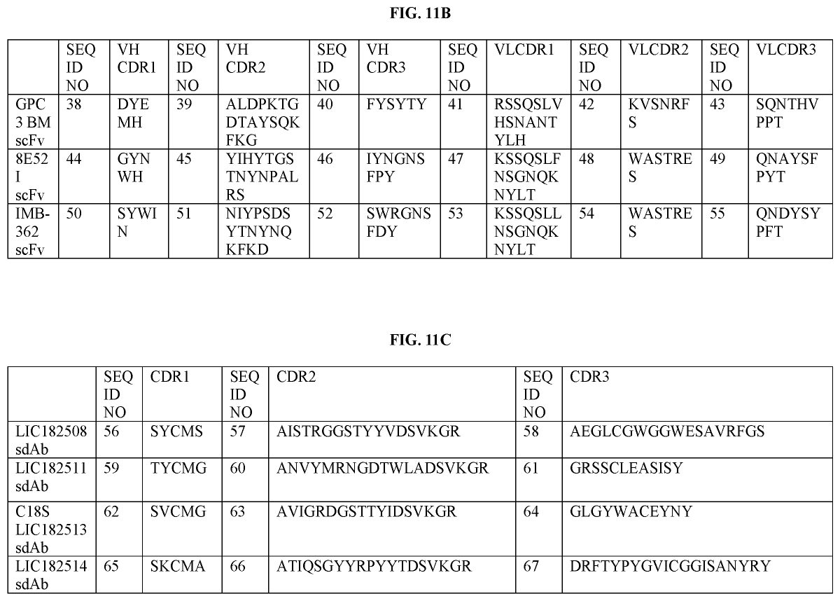

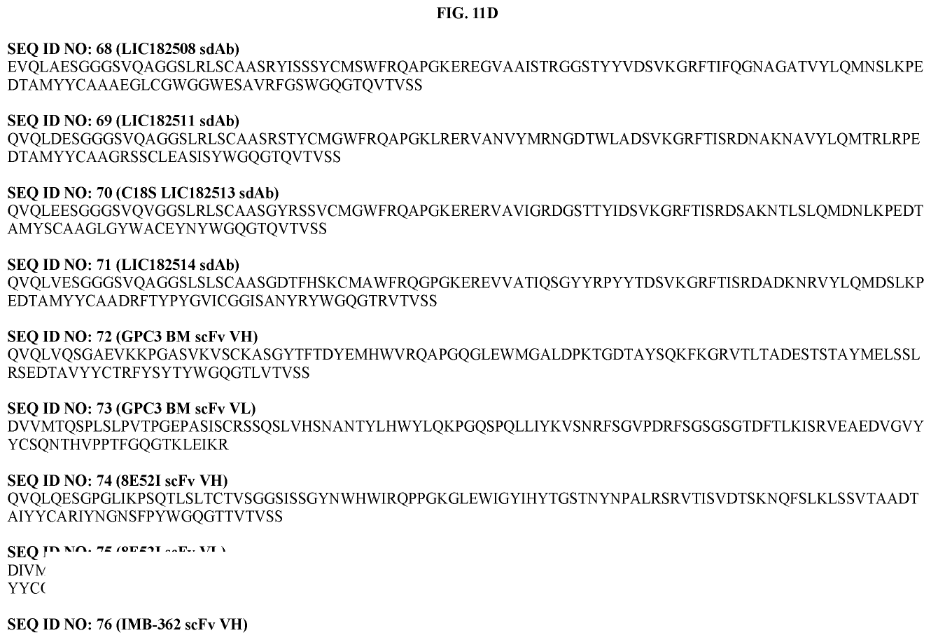

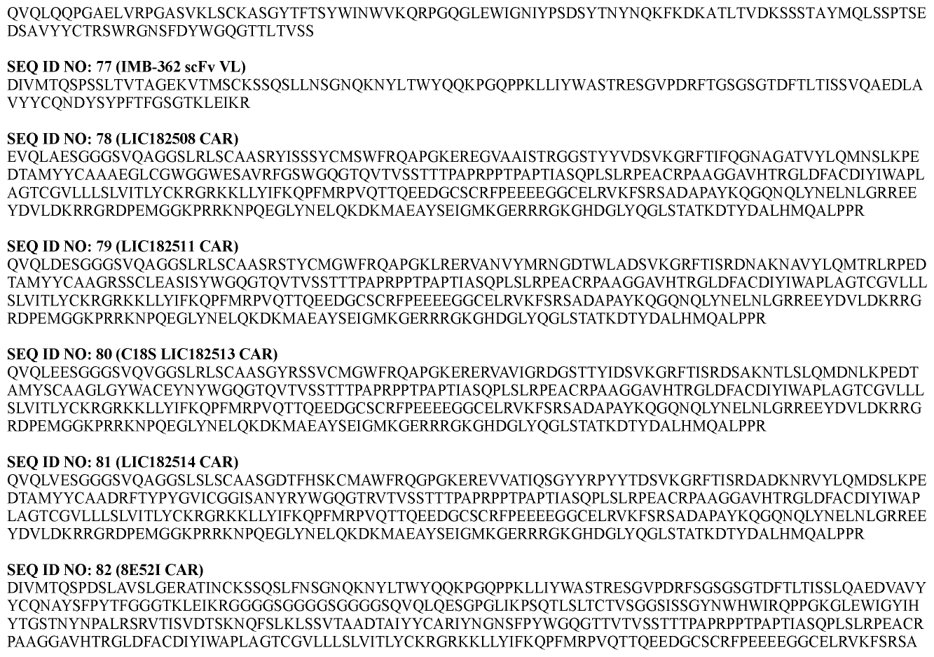

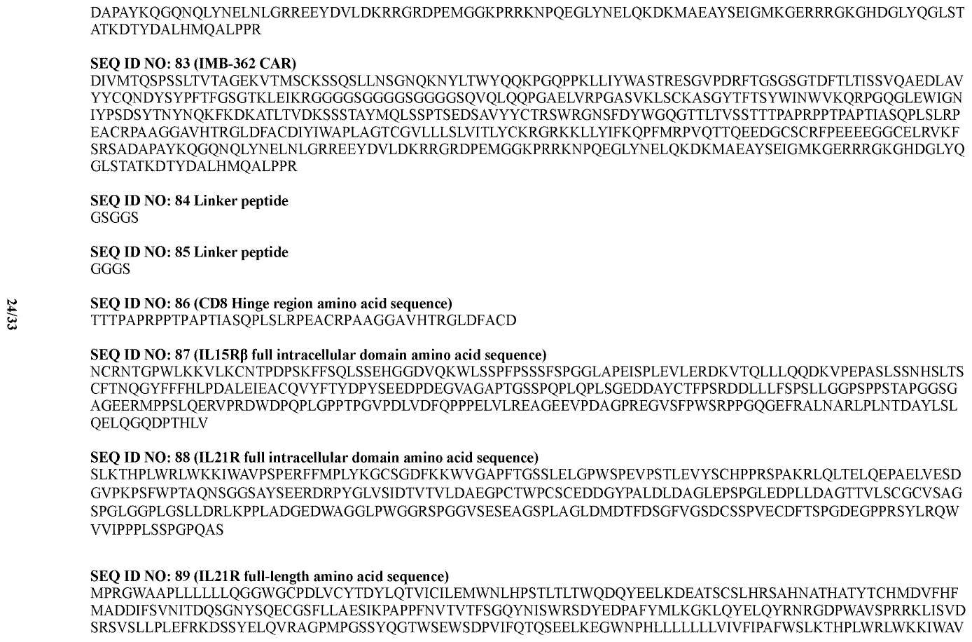

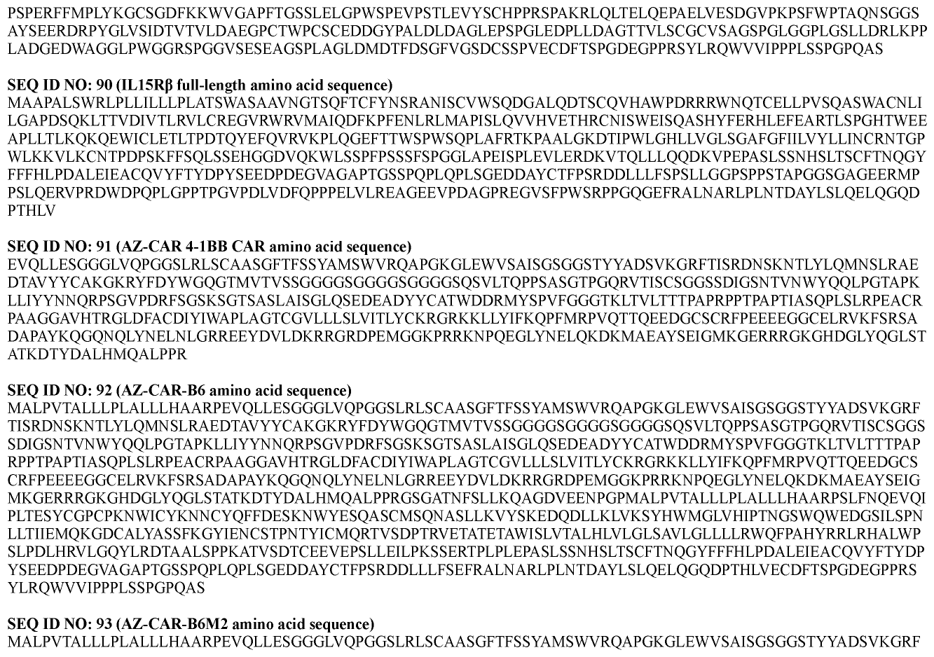

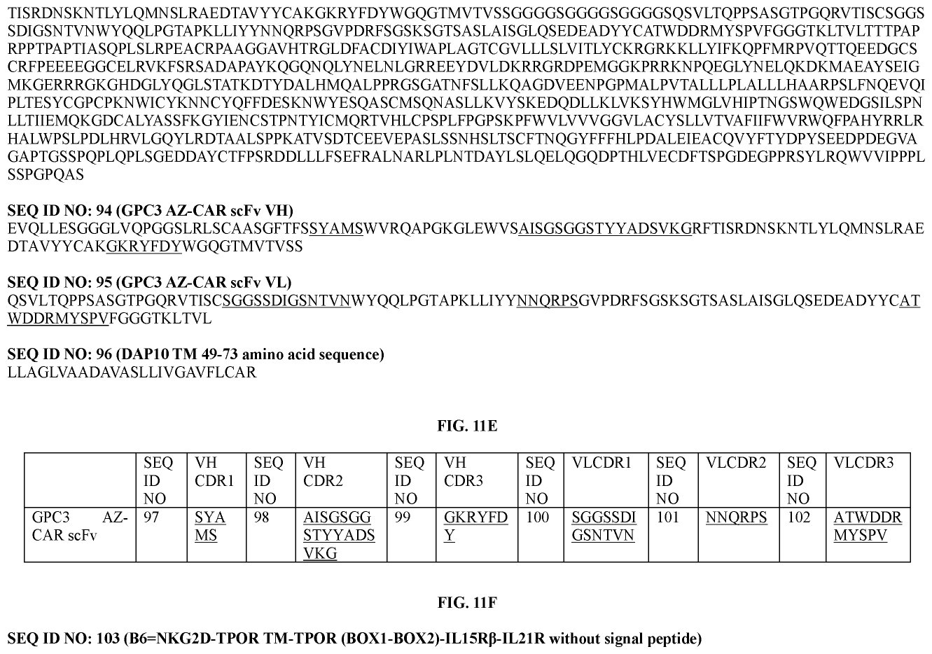

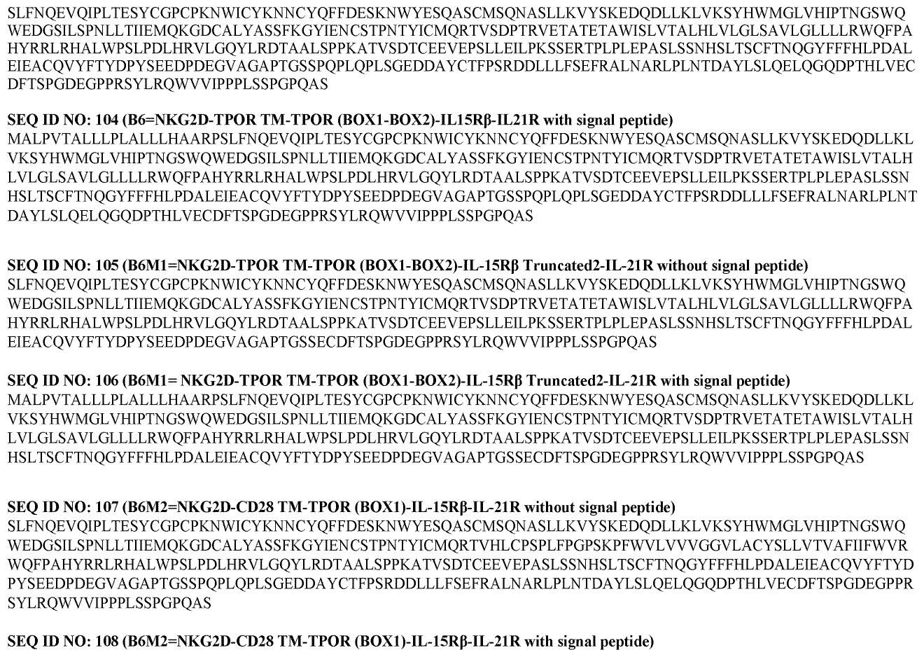

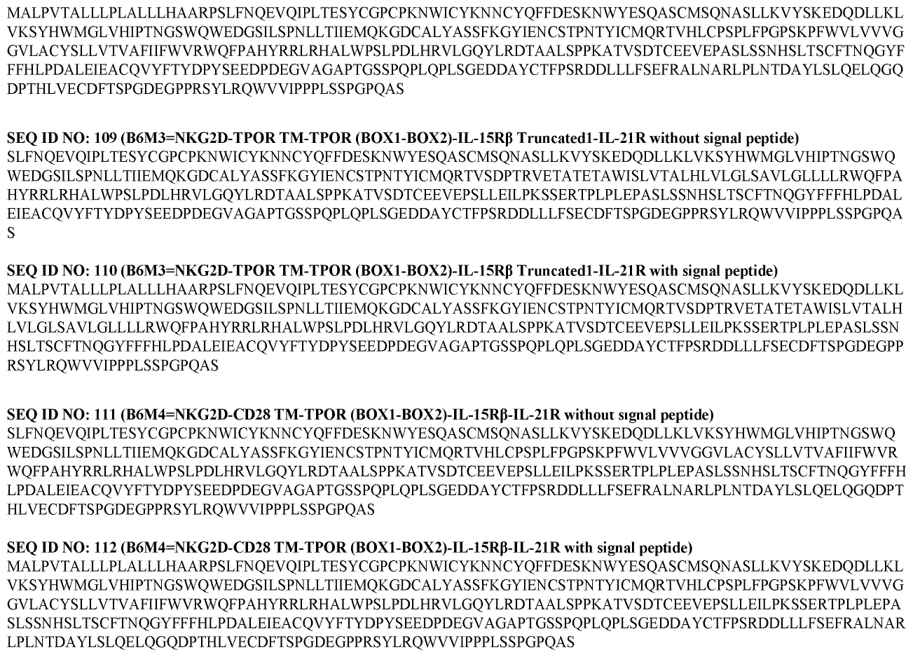

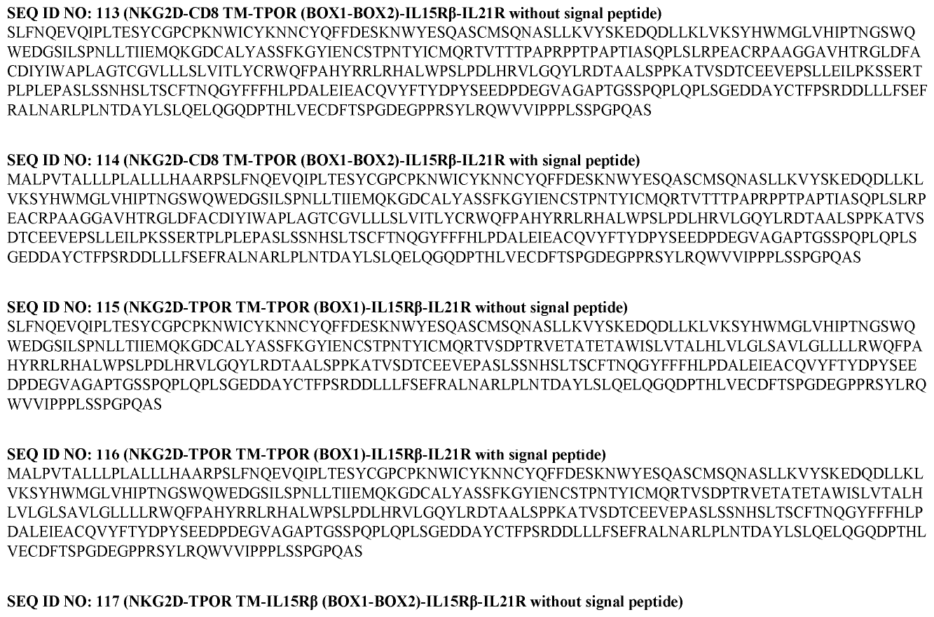

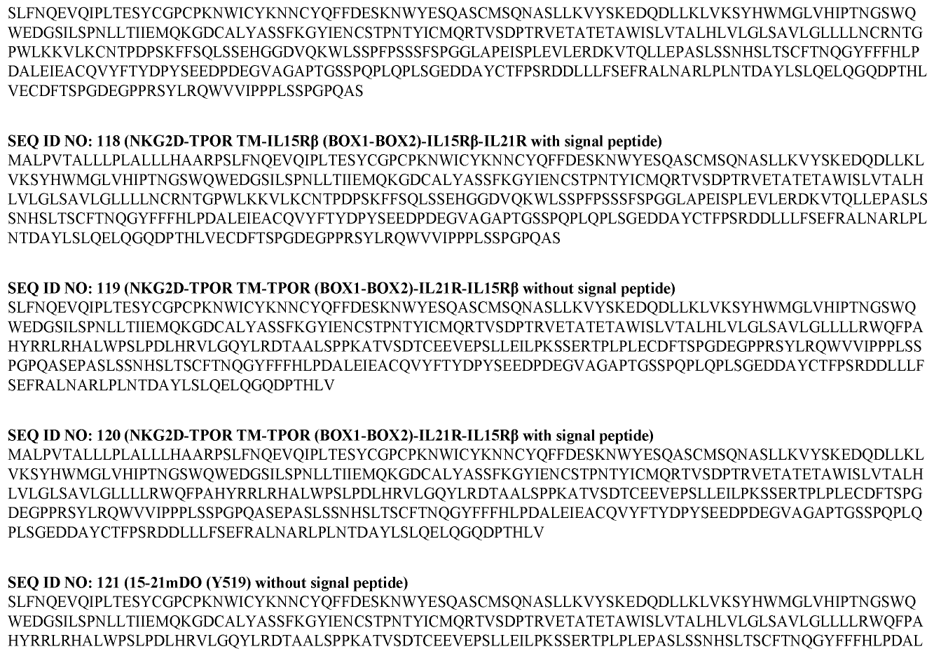

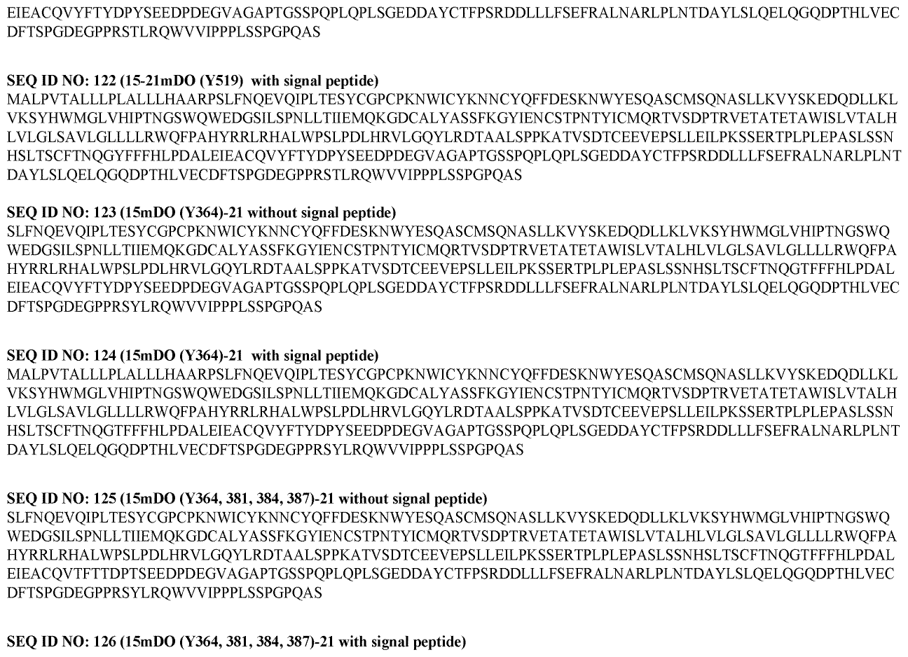

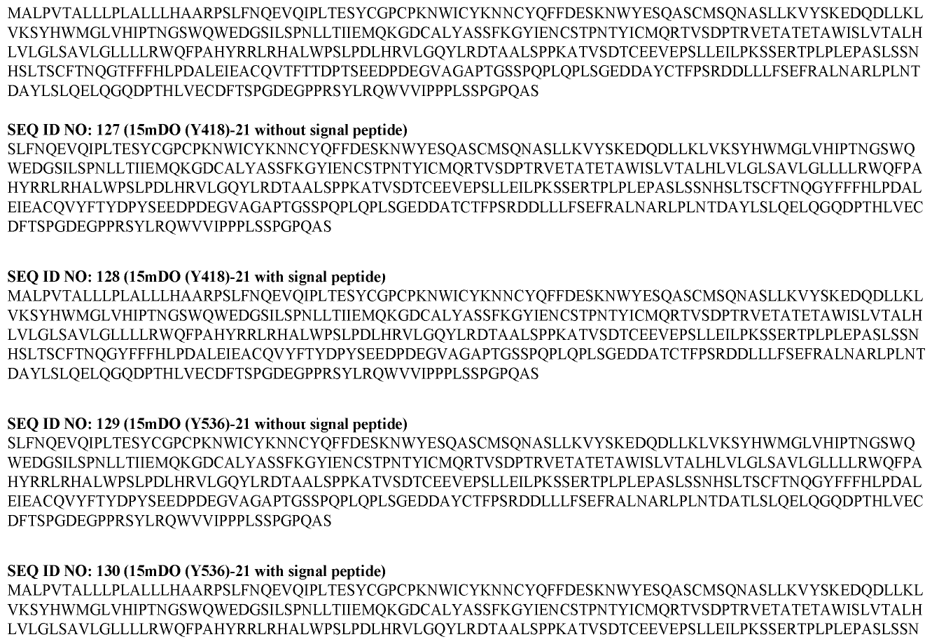

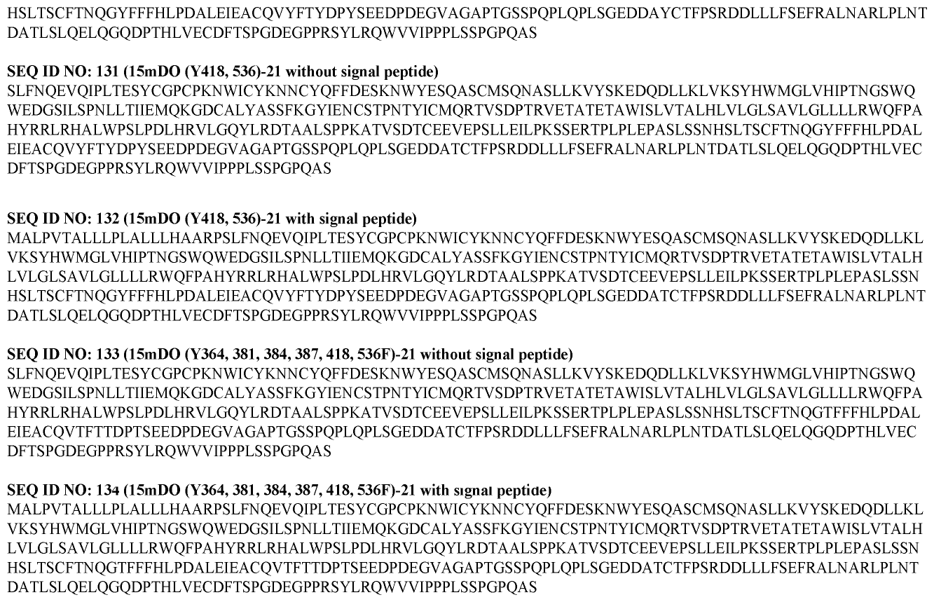

FIG. 10A and FIG. 10B show the long-term killing efficacy and proliferation of GPC3 CAR γδ T cells against Huh7 cells, respectively. AZ-CAR γδ T cells armored with NKG2D chimeric cytokine receptor using B6 or B6M2 enhanced CAR γδ T cells cytotoxicity and proliferation compared to naked CAR γδ T cells in vitro, and AZ-CAR γδ T cells armored with B6M2 were slightly superior to that armored with B6 in improving cytotoxicity and proliferation in vitro, which reveals that NKG2D chimeric cytokine receptor potency was independent on a specific binder.

In summary, NKG2D chimeric receptor armored CAR-γδ T cells provided was efficacious and safe in treating tumors as demonstrated via in vitro efficacy and in vivo efficacy and safety tests.

### OTHER EMBODIMENTS

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

What is claimed is:

1. A chimeric cytokine receptor comprising:
(a) a first extracellular antigen binding domain,
(b) a first transmembrane domain,
(c) a Janus Kinase (JAK)-binding domain comprising a TPOR BOX1 motif, wherein optionally the JAK-binding domain does not comprise a TPOR BOX2 motif, and
(d) a cytokine receptor intracellular domain.

2. The chimeric cytokine receptor of claim 1, wherein the first transmembrane domain is a TPOR transmembrane domain, a CD28 transmembrane domain, a CD8 transmembrane domain or a DAP10 transmembrane domain.

3. The chimeric cytokine receptor of claim 1 or 2, wherein the JAK-binding domain comprises an amino acid sequence set forth in SEQ ID NO: 8 or an amino acid sequence that is at least 80%, 85%, 90%, 95% or 99% identical to the amino acid sequence set forth in SEQ ID NO: 8.

4. A chimeric cytokine receptor comprising:
(a) a first extracellular antigen binding domain,
(b) a first transmembrane domain, wherein the first transmembrane domain is selected from a CD28 transmembrane domain, a CD8 transmembrane domain and a DAP10 transmembrane domain,
(c) a JAK-binding domain, and
(d) a cytokine receptor intracellular domain.

5. The chimeric cytokine receptor of claim 4, wherein the JAK-binding domain comprises a TPOR BOX1 motif and a TPOR BOX2 motif.

6. The chimeric cytokine receptor of claim 5, wherein the JAK-binding domain comprises an amino acid sequence set forth in SEQ ID NO: 7 or an amino acid sequence that is at least 80%, 85%, 90%, 95% or 99% identical to the amino acid sequence set forth in SEQ ID NO: 7.

7. The chimeric cytokine receptor of claim 4, wherein the JAK-binding domain comprises a TPOR BOX1 motif, and wherein the JAK-binding domain does not comprise a TPOR BOX2 motif.

8. The chimeric cytokine receptor of claim 7, wherein the JAK-binding domain comprises an amino acid sequence set forth in SEQ ID NO: 8 or an amino acid sequence that is at least 80%, 85%, 90%, 95% or 99% identical to the amino acid sequence set forth in SEQ ID NO: 8.

9. The chimeric cytokine receptor of any one of claims 1-8, wherein the cytokine receptor intracellular domain comprises a STAT-binding domain, wherein optionally the STAT-binding domain comprises an IL-21R intracellular domain and/or an IL-15Rβ intracellular domain.

10. The chimeric cytokine receptor of claim 9, wherein the IL-21R intracellular domain comprises an amino acid sequence set forth in SEQ ID NO: 20 or 21 or an amino acid sequence that is at least 80%, 85%, 90%, 95% or 99% identical to the amino acid sequence set forth in SEQ ID NO: 20 or 21.

11. The chimeric cytokine receptor of claim 9, wherein the IL-15Rβ intracellular domain comprises an amino acid sequence set forth in any one of SEQ ID NOs: 10-18 or an amino acid sequence that is at least 80%, 85%, 90%, 95% or 99% identical to the amino acid sequence set forth in any one of SEQ ID NOs: 10-18.

12. The chimeric cytokine receptor of any one of claims 1-11, wherein the first extracellular antigen binding domain binds to an antigen expressed on the surface of a tumor cell.

13. The chimeric cytokine receptor of any one of claims 1-12, wherein the first extracellular antigen binding domain is derived from NKG2D, truncated NKG2D, a NKG2D extracellular domain, an antibody or antigen binding fragment thereof targeting NKG2D ligands, TIGIT or SIRP-α, or a variant thereof.

14. The chimeric cytokine receptor of claim 12 or 13, wherein the first extracellular antigen binding domain is derived from NKG2D or truncated NKG2D, or a variant thereof, wherein optionally the first extracellular antigen binding domain is derived from the extracellular domain (ECD) of NKG2D or truncated NKG2D, and wherein optionally the first extracellular antigen binding domain comprises the amino acid sequence of SEQ ID NO: 6, or an amino acid sequence having at least 75%, 80%, 85%, 90%, 91%, 92%, 93%, 94%, 95%, 96%, 97%, 98%, or 99% identity to SEQ ID NO: 6.

15. The chimeric cytokine receptor of claim 13 or 14, wherein the chimeric cytokine receptor comprises the amino acid sequence of SEQ ID NO: 103, 105, 107, 109 or 111, or an amino acid sequence having at least 75%, 80%, 85%, 90%, 91%, 92%, 93%, 94%, 95%, 96%, 97%, 98%, or 99% identity to SEQ ID NO: 103, 105, 107, 109 or 111.

16. A cell comprising the chimeric cytokine receptor of any one of claims 1-15.

17. The cell of claim 16, wherein the cell further comprises a functional exogenous receptor.

18. The cell of claim 17, wherein the functional exogenous receptor comprising: (a) a second extracellular antigen binding domain, (b) a second transmembrane domain, and (c) an intracellular signaling domain.

19. The cell of claim 18, wherein the functional exogenous receptor is a T cell receptor (TCR), a chimeric antigen receptor (CAR), a chimeric TCR (cTCR), or a T cell antigen coupler (TAC)-like chimeric receptor.

20. The cell of claim 19, wherein the functional exogenous receptor is a CAR, wherein optionally the CAR is a single CAR, dual CAR, tandem CAR or split CAR.

21. The cell of claim 19 or 20, wherein the CAR binds to a tumor-associated antigen, wherein optionally the tumor-associated antigen is selected from the group consisting of CD19, AFP, BCMA, NY-ESO-1, VEGFR2, MAGE-A3, CD20, CD22, CD30, CD33, GUCY2C, MUC17, FGFR2, CLL1, CD38, CEA, CS1, CD138, CD123/IL3Rα, c-Met, gp100, MUC1, IGF-I receptor, EpCAM, CEA, EGFR (such as EGFRVIII), GD2, HER2, IGF1R, mesothelin, PSMA, ROR1, WT1, Glypican 3 (GPC3), Guanylate cyclase 2C (GCC), DLL3, Claudin18.2, Claudin6, Glycolipid F77, PD-L1, PD-L2, and other tumor antigens with clinical significance, and combinations thereof.

22. The cell of any one of claims 19-21, wherein the CAR comprises a single chain variable fragment (scFv) comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 38, 39, 40, respectively, and the selected VL CDRs 1, 2, 3 amino acid sequences are set forth in SEQ ID NOs: 41, 42, 43, respectively;
(2) the selected VH CDRs 1, 2, 3 amino acid sequences are set forth in SEQ ID NOs: 44, 45, 46, respectively, and the selected VL CDRs 1, 2, 3 amino acid sequences are set forth in SEQ ID NOs: 47, 48, 49, respectively;
(3) the selected VH CDRs 1, 2, 3 amino acid sequences are set forth in SEQ ID NOs: 50, 51, 52, respectively, and the selected VL CDRs 1, 2, 3 amino acid sequences are set forth in SEQ ID NOs: 53, 54, 55, respectively; and
(4) the selected VH CDRs 1, 2, 3 amino acid sequences are set forth in SEQ ID NOs: 97, 98, 99, respectively, and the selected VL CDRs 1, 2, 3 amino acid sequences are set forth in SEQ ID NOs: 100, 101, 102, respectively.

23. The cell of claim 22, wherein the CAR comprises a heavy chain variable region (VH) comprising an amino acid sequence that is at least 80% identical to a selected VH sequence, and a light chain variable region (VL) comprising an amino acid sequence that is at least 80% identical to a selected VL sequence, wherein the selected VH sequence and the selected VL sequence are one of the following:
(1) the selected VH sequence is SEQ ID NO: 72, and the selected VL sequence is SEQ ID NO: 73;
(2) the selected VH sequence is SEQ ID NO: 74, and the selected VL sequence is SEQ ID NO: 75;
(3) the selected VH sequence is SEQ ID NO: 76, and the selected VL sequence is SEQ ID NO: 77; and
(4) the selected VH sequence is SEQ ID NO: 94, and the selected VL sequence is SEQ ID NO: 95.

24. The cell of any one of claims 19-21, wherein the CAR comprises a V_{H}H antibody moiety that having the amino acid sequences of:
(1) a CDR1 comprising the amino acid sequence of SEQ ID NO: 56, a CDR2 comprising the amino acid sequence of SEQ ID NO: 57, and a CDR3 comprising the amino acid sequence of SEQ ID NO: 58;
(2) a CDR1 comprising the amino acid sequence of SEQ ID NO: 59, a CDR2 comprising the amino acid sequence of SEQ ID NO: 60, and a CDR3 comprising the amino acid sequence of SEQ ID NO: 61;
(3) a CDR1 comprising the amino acid sequence of SEQ ID NO: 62, a CDR2 comprising the amino acid sequence of SEQ ID NO: 63, and a CDR3 comprising the amino acid sequence of SEQ ID NO: 64; or
(4) a CDR1 comprising the amino acid sequence of SEQ ID NO: 65, a CDR2 comprising the amino acid sequence of SEQ ID NO: 66, and a CDR3 comprising the amino acid sequence of SEQ ID NO: 67.

25. The cell of claim 24, wherein the CAR comprises a V_{H}H antibody moiety comprising the amino acid sequences of any one of SEQ ID NOs: 68-71 or an amino acid sequence having at least 90%, 95%, or 99% identity to the amino acid sequence of any one of SEQ ID NOs: 68-71.

26. The cell of any one of claims 18-25, wherein the intracellular signaling domain comprises a primary intracellular signaling domain of a cell, wherein optionally the primary intracellular signaling domain is from CD34.

27. The cell of any one of claims 18-26, wherein the intracellular signaling domain comprises a co-stimulatory signaling domain, wherein optionally the co-stimulatory signaling domain is derived from a co-stimulatory molecule selected from the group consisting of CD27, CD28, CD137, OX40, CD30, CD40, CD3, LFA-1, ICOS, CD2, CD7, LIGHT, NKG2C, B7-H3, ligands of CD83 and combinations thereof.

28. The cell of any one of claims 19-27, wherein the CAR comprises an amino acid sequence set forth in any one of SEQ ID NOs: 4, 78-83 and 91, or an amino acid sequence having at least 90%, 95%, or 99% identity to the amino acid sequence set forth in any one of SEQ ID NOs: 4, 78-83 and 91.

29. The cell of any one of claims 17-28, wherein the chimeric cytokine receptor and the functional exogenous receptor are linked to each other via a peptide linker.

30. The cell of claim 29, wherein the peptide linker is a self-cleaving peptide linker, wherein optionally the self-cleaving peptide linker is a 2A self-cleaving peptide, wherein optionally the 2A self-cleaving peptide is selected from a group consisting of F2A, E2A, P2A, T2A, and variants thereof.

31. The cell of claim 29 or 30, wherein the cell comprises the amino acid sequence set forth in any one of SEQ ID NOs: 22-37, 92 and 93, or an amino acid sequence that is at least 80%, 85%, 90%, 95% or 99% identical to the amino acid sequence set forth in any one of SEQ ID NOs: 22-37, 92 and 93.

32. The cell of any one of claims 16-31, wherein the cell is an immune cell.

33. The cell of claim 32, wherein the immune cell is selected from a group consisting of T cell, NK cell, NKT, peripheral blood mononuclear cell (PBMC), hematopoietic stem cell, pluripotent stem cell, an embryonic stem cell, a macrophage, a monocyte, a neutrophil, an eosinophil and a combination thereof.

34. The cell of claim 33, wherein the T cell is a αβT cell, γδT cell or a combination of αβT and γδT cell.

35. The cell of claim 33, wherein the T cell, upon activation, exhibits increased expression of pSTAT3 and pSTAT5.

36. A nucleic acid comprising a nucleic acid sequence encoding the chimeric cytokine receptor of any one of claims 1-15.

37. The nucleic acid of claim 36, wherein the nucleic acid further comprises a nucleic acid sequence encoding a functional exogenous receptor comprising: (a) a second extracellular antigen binding domain, (b) a second transmembrane domain, and (c) an intracellular signaling domain.

38. The nucleic acid of claim 37, wherein the functional exogenous receptor is a T cell receptor (TCR), a chimeric antigen receptor (CAR), a chimeric TCR (cTCR), or a T cell antigen coupler (TAC)-like chimeric receptor.

39. The nucleic acid of claim 38, wherein the CAR binds to a tumor-associated antigen, wherein optionally the tumor-associated antigen is selected from the group consisting of CD19, AFP, BCMA, NY-ESO-1, VEGFR2, MAGE-A3, CD20, CD22, CD30, CD33, GUCY2C, MUC17, FGFR2, CLL1, CD38, CEA, CS1, CD138, CD123/IL3Rα, c-Met, gp100, MUC1, IGF-I receptor, EpCAM, CEA, EGFR (such as EGFRvIII), GD2, HER2, IGF1R, mesothelin, PSMA, ROR1, WT1, Glypican 3 (GPC3), Guanylate cyclase 2C (GCC), DLL3, Claudin18.2, Claudin6, Glycolipid F77, PD-L1, PD-L2, and other tumor antigens with clinical significance, and combinations thereof.

40. The nucleic acid of claim 38 or 39, wherein the CAR comprises a single chain variable fragment (scFv) comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 38, 39, 40, respectively, and the selected VL CDRs 1, 2, 3 amino acid sequences are set forth in SEQ ID NOs: 41, 42, 43, respectively;
(2) the selected VH CDRs 1, 2, 3 amino acid sequences are set forth in SEQ ID NOs: 44, 45, 46, respectively, and the selected VL CDRs 1, 2, 3 amino acid sequences are set forth in SEQ ID NOs: 47, 48, 49, respectively;
(3) the selected VH CDRs 1, 2, 3 amino acid sequences are set forth in SEQ ID NOs: 50, 51, 52, respectively, and the selected VL CDRs 1, 2, 3 amino acid sequences are set forth in SEQ ID NOs: 53, 54, 55, respectively; and
(4) the selected VH CDRs 1, 2, 3 amino acid sequences are set forth in SEQ ID NOs: 97, 98, 99, respectively, and the selected VL CDRs 1, 2, 3 amino acid sequences are set forth in SEQ ID NOs: 100, 101, 102, respectively.

41. The nucleic acid of claim 40, wherein the CAR comprises a heavy chain variable region (VH) comprising an amino acid sequence that is at least 80% identical to a selected VH sequence, and a light chain variable region (VL) comprising an amino acid sequence that is at least 80% identical to a selected VL sequence, wherein the selected VH sequence and the selected VL sequence are one of the following:
(1) the selected VH sequence is SEQ ID NO: 72, and the selected VL sequence is SEQ ID NO: 73;
(2) the selected VH sequence is SEQ ID NO: 74, and the selected VL sequence is SEQ ID NO: 75;
(3) the selected VH sequence is SEQ ID NO: 76, and the selected VL sequence is SEQ ID NO: 77; and
(4) the selected VH sequence is SEQ ID NO: 94, and the selected VL sequence is SEQ ID NO: 95.

42. The nucleic acid of claim 38 or 39, wherein the CAR comprises a V_{H}H antibody moiety that having the amino acid sequences of:
(1) a CDR1 comprising the amino acid sequence of SEQ ID NO: 56, a CDR2 comprising the amino acid sequence of SEQ ID NO: 57, and a CDR3 comprising the amino acid sequence of SEQ ID NO: 58;
(2) a CDR1 comprising the amino acid sequence of SEQ ID NO: 59, a CDR2 comprising the amino acid sequence of SEQ ID NO: 60, and a CDR3 comprising the amino acid sequence of SEQ ID NO: 61;
(3) a CDR1 comprising the amino acid sequence of SEQ ID NO: 62, a CDR2 comprising the amino acid sequence of SEQ ID NO: 63, and a CDR3 comprising the amino acid sequence of SEQ ID NO: 64; or
(4) a CDR1 comprising the amino acid sequence of SEQ ID NO: 65, a CDR2 comprising the amino acid sequence of SEQ ID NO: 66, and a CDR3 comprising the amino acid sequence of SEQ ID NO: 67.

43. The nucleic acid of claim 42, wherein the CAR comprises a V_{H}H antibody moiety comprising an amino acid sequence of any one of SEQ ID NOs: 68-71 or an amino acid sequence having at least 90%, 95%, or 99% identity to the amino acid sequence of any one of SEQ ID NOs: 68-71.

44. The nucleic acid of any one of claims 37-43, wherein the intracellular signaling domain further comprises a co-stimulatory signaling domain.

45. The nucleic acid of any one of claims 38-44, wherein the CAR comprises an amino acid sequence set forth in any one of SEQ ID NOs: 4, 78-83 and 91, or an amino acid sequence having at least 90%, 95%, or 99% identity to the amino acid sequence set forth in any one of SEQ ID NOs: 4, 78-83 and 91.

46. The nucleic acid of any one of claims 37-45, wherein the chimeric cytokine receptor nucleic acid sequence and the nucleic acid sequence encoding the functional exogenous receptor are linked to each other via a third nucleic acid sequence encoding a peptide linker.

47. The nucleic acid of claim 46, wherein the nucleic acid comprises a nucleic acid sequence encoding an amino acid sequence set forth in any one of SEQ ID NOs: 22-37, 92 and 93, or an amino acid sequence that is at least 80%, 85%, 90%, 95% or 99% identical to the amino acid sequence set forth in any one of SEQ ID NOs: 22-37, 92 and 93.

48. A vector comprising the nucleic acid of any one of claims 36-47.

49. A pharmaceutical composition, comprising the chimeric cytokine receptor of any one of claims 1-15, the cell of any one of claims 16-35, or the nucleic acid of any one of claims 36-47 and a pharmaceutically acceptable carrier.

50. A method of treating a disease or disorder in a subject, comprising administering to the subject an effective amount of the pharmaceutical composition of claim 49.

51. The method of claim 50, wherein the disease or disorder is a cancer, an inflammatory or autoimmune disease.

52. The method of claim 51, wherein the cancer is solid cancer or hematologic cancer.

53. The method of claim 52, wherein the cancer is liver cancer, gastric cancer, colon cancer, lymphoma, acute myeloid leukemia (AML) or chronic myelogenous leukemia (CML).


SEQUENCE LISTING (134 sequences)

SEQ ID NO: 1 - protein, 21 aa, synthetic construct
  source 1..21: note = CD8alpha signal peptide amino acid sequence
MALPVTALLLPLALLLHAARP

SEQ ID NO: 2 - protein, 24 aa, synthetic construct
  source 1..24: note = CD8 TM amino acid sequence
IYIWAPLAGTCGVLLLSLVITLYC

SEQ ID NO: 3 - protein, 41 aa, synthetic construct
  source 1..41: note = CD28 TM amino acid sequence
HLCPSPLFPGPSKPFWVLVVVGGVLACYSLLVTVAFIIFWV

SEQ ID NO: 4 - protein, 466 aa, synthetic construct
  source 1..466: note = BM 4-1BB CAR amino acid sequence
DVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 22 aa, synthetic construct
  source 1..22: note = P2A amino acid sequence
GSGATNFSLLKQAGDVEENPGP

SEQ ID NO: 6 - protein, 136 aa, synthetic construct
  source 1..136: note = NKG2D 81-216 amino acid sequence
SLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 7 - protein, 69 aa, synthetic construct
  source 1..69: note = TPOR BOX1-BOX2 514-582 amino acid sequence
RWQFPAHYRRLRHALWPSLPDLHRVLGQYLRDTAALSPPKATVSDTCEEVEPSLLEILPKSSERTPLPL

SEQ ID NO: 8 - protein, 50 aa, synthetic construct
  source 1..50: note = TPOR BOX1 514-563 amino acid sequence
RWQFPAHYRRLRHALWPSLPDLHRVLGQYLRDTAALSPPKATVSDTCEEV

SEQ ID NO: 9 - protein, 36 aa, synthetic construct
  source 1..36: note = TPOR TM 478-513 amino acid sequence
SDPTRVETATETAWISLVTALHLVLGLSAVLGLLLL

SEQ ID NO: 10 - protein, 118 aa, synthetic construct
  source 1..118: note = IL-15Rbeta 345-431, 521-551 amino acid sequence
EPASLSSNHSLTSCFTNQGYFFFHLPDALEIEACQVYFTYDPYSEEDPDEGVAGAPTGSSPQPLQPLSGEDDAYCTFPSRDDLLLFSEFRALNARLPLNTDAYLSLQELQGQDPTHLV

SEQ ID NO: 11 - protein, 118 aa, synthetic construct
  source 1..118: note = 15mDO Y364 amino acid sequence
EPASLSSNHSLTSCFTNQGTFFFHLPDALEIEACQVYFTYDPYSEEDPDEGVAGAPTGSSPQPLQPLSGEDDAYCTFPSRDDLLLFSEFRALNARLPLNTDAYLSLQELQGQDPTHLV

SEQ ID NO: 12 - protein, 118 aa, synthetic construct
  source 1..118: note = 15mDO Y364, 381, 384, 387 amino acid sequence
EPASLSSNHSLTSCFTNQGTFFFHLPDALEIEACQVTFTTDPTSEEDPDEGVAGAPTGSSPQPLQPLSGEDDAYCTFPSRDDLLLFSEFRALNARLPLNTDAYLSLQELQGQDPTHLV

SEQ ID NO: 13 - protein, 118 aa, synthetic construct
  source 1..118: note = 15mDO Y418 amino acid sequence
EPASLSSNHSLTSCFTNQGYFFFHLPDALEIEACQVYFTYDPYSEEDPDEGVAGAPTGSSPQPLQPLSGEDDATCTFPSRDDLLLFSEFRALNARLPLNTDAYLSLQELQGQDPTHLV

SEQ ID NO: 14 - protein, 118 aa, synthetic construct
  source 1..118: note = 15mDO Y536 amino acid sequence
EPASLSSNHSLTSCFTNQGYFFFHLPDALEIEACQVYFTYDPYSEEDPDEGVAGAPTGSSPQPLQPLSGEDDAYCTFPSRDDLLLFSEFRALNARLPLNTDATLSLQELQGQDPTHLV

SEQ ID NO: 15 - protein, 118 aa, synthetic construct
  source 1..118: note = 15mDO Y418, 536 amino acid sequence
EPASLSSNHSLTSCFTNQGYFFFHLPDALEIEACQVYFTYDPYSEEDPDEGVAGAPTGSSPQPLQPLSGEDDATCTFPSRDDLLLFSEFRALNARLPLNTDATLSLQELQGQDPTHLV

SEQ ID NO: 16 - protein, 118 aa, synthetic construct
  source 1..118: note = 15mDO Y364, 381, 384, 387, 418, 536 amino acid sequence
EPASLSSNHSLTSCFTNQGTFFFHLPDALEIEACQVTFTTDPTSEEDPDEGVAGAPTGSSPQPLQPLSGEDDATCTFPSRDDLLLFSEFRALNARLPLNTDATLSLQELQGQDPTHLV

SEQ ID NO: 17 - protein, 87 aa, synthetic construct
  source 1..87: note = IL-15Rbeta truncated1 345-431 amino acid sequence
EPASLSSNHSLTSCFTNQGYFFFHLPDALEIEACQVYFTYDPYSEEDPDEGVAGAPTGSSPQPLQPLSGEDDAYCTFPSRDDLLLFS

SEQ ID NO: 18 - protein, 60 aa, synthetic construct
  source 1..60: note = IL-15Rbeta truncated2 345-404 amino acid sequence
EPASLSSNHSLTSCFTNQGYFFFHLPDALEIEACQVYFTYDPYSEEDPDEGVAGAPTGSS

SEQ ID NO: 19 - protein, 72 aa, synthetic construct
  source 1..72: note = IL-15Rbeta BOX1-BOX2 266-337 amino acid sequence
NCRNTGPWLKKVLKCNTPDPSKFFSQLSSEHGGDVQKWLSSPFPSSSFSPGGLAPEISPLEVLERDKVTQLL

SEQ ID NO: 20 - protein, 35 aa, synthetic construct
  source 1..35: note = IL-21R 504-538 amino acid sequence
ECDFTSPGDEGPPRSYLRQWVVIPPPLSSPGPQAS

SEQ ID NO: 21 - protein, 35 aa, synthetic construct
  source 1..35: note = 21mDO Y519 amino acid sequence
ECDFTSPGDEGPPRSTLRQWVVIPPPLSSPGPQAS

SEQ ID NO: 22 - protein, 924 aa, synthetic construct
  source 1..924: note = BM-NKG2D-TPOR TM-TPOR (BOX1-BOX2)-IL15Rbeta-IL21R= BM-15-21 amino acid sequence
MALPVTALLLPLALLLHAARPDVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLEPASLSSNHSLTSCFTNQGYFFFHLPDALEIEACQVYFTYDPYSEEDPDEGVAGAPTGSSPQPLQPLSGEDDAYCTFPSRDDLLLFSEFRALNARLPLNTDAYLSLQELQGQDPTHLVECDFTSPGDEGPPRSYLRQWVVIPPPLSSPGPQAS

SEQ ID NO: 23 - protein, 957 aa, synthetic construct
  source 1..957: note = BM-NKG2D-CD8 TM-TPOR (BOX1-BOX2)-IL15Rbeta-IL21R amino acid sequence
MALPVTALLLPLALLLHAARPDVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCRWQFPAHYRRLRHALWPSLPDLHRVLGQYLRDTAALSPPKATVSDTCEEVEPSLLEILPKSSERTPLPLEPASLSSNHSLTSCFTNQGYFFFHLPDALEIEACQVYFTYDPYSEEDPDEGVAGAPTGSSPQPLQPLSGEDDAYCTFPSRDDLLLFSEFRALNARLPLNTDAYLSLQELQGQDPTHLVECDFTSPGDEGPPRSYLRQWVVIPPPLSSPGPQAS

SEQ ID NO: 24 - protein, 929 aa, synthetic construct
  source 1..929: note = BM-NKG2D-CD28 TM-TPOR (BOX1-BOX2)-IL15Rbeta-IL21R amino acid sequence
MALPVTALLLPLALLLHAARPDVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVHLCPSPLFPGPSKPFWVLVVVGGVLACYSLLVTVAFIIFWVRWQFPAHYRRLRHALWPSLPDLHRVLGQYLRDTAALSPPKATVSDTCEEVEPSLLEILPKSSERTPLPLEPASLSSNHSLTSCFTNQGYFFFHLPDALEIEACQVYFTYDPYSEEDPDEGVAGAPTGSSPQPLQPLSGEDDAYCTFPSRDDLLLFSEFRALNARLPLNTDAYLSLQELQGQDPTHLVECDFTSPGDEGPPRSYLRQWVVIPPPLSSPGPQAS

SEQ ID NO: 25 - protein, 905 aa, synthetic construct
  source 1..905: note = BM-NKG2D-TPOR TM-TPOR (BOX1)-IL15Rbeta-IL21R amino acid sequence
MALPVTALLLPLALLLHAARPDVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLRWQFPAHYRRLRHALWPSLPDLHRVLGQYLRDTAALSPPKATVSDTCEEVEPASLSSNHSLTSCFTNQGYFFFHLPDALEIEACQVYFTYDPYSEEDPDEGVAGAPTGSSPQPLQPLSGEDDAYCTFPSRDDLLLFSEFRALNARLPLNTDAYLSLQELQGQDPTHLVECDFTSPGDEGPPRSYLRQWVVIPPPLSSPGPQAS

SEQ ID NO: 26 - protein, 927 aa, synthetic construct
  source 1..927: note = BM-NKG2D-TPOR TM-IL15Rbeta (BOX1-BOX2)-IL15Rbeta-IL21R amino acid sequence
MALPVTALLLPLALLLHAARPDVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLNCRNTGPWLKKVLKCNTPDPSKFFSQLSSEHGGDVQKWLSSPFPSSSFSPGGLAPEISPLEVLERDKVTQLLEPASLSSNHSLTSCFTNQGYFFFHLPDALEIEACQVYFTYDPYSEEDPDEGVAGAPTGSSPQPLQPLSGEDDAYCTFPSRDDLLLFSEFRALNARLPLNTDAYLSLQELQGQDPTHLVECDFTSPGDEGPPRSYLRQWVVIPPPLSSPGPQAS

SEQ ID NO: 27 - protein, 910 aa, synthetic construct
  source 1..910: note = BM-NKG2D-CD28 TM-TPOR (BOX1)-IL15Rbeta-IL21R amino acid sequence
MALPVTALLLPLALLLHAARPDVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVHLCPSPLFPGPSKPFWVLVVVGGVLACYSLLVTVAFIIFWVRWQFPAHYRRLRHALWPSLPDLHRVLGQYLRDTAALSPPKATVSDTCEEVEPASLSSNHSLTSCFTNQGYFFFHLPDALEIEACQVYFTYDPYSEEDPDEGVAGAPTGSSPQPLQPLSGEDDAYCTFPSRDDLLLFSEFRALNARLPLNTDAYLSLQELQGQDPTHLVECDFTSPGDEGPPRSYLRQWVVIPPPLSSPGPQAS

SEQ ID NO: 28 - protein, 924 aa, synthetic construct
  source 1..924: note = BM-NKG2D-TPOR TM-TPOR (BOX1-BOX2)-IL21R-IL15Rbeta amino acid sequence
MALPVTALLLPLALLLHAARPDVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLECDFTSPGDEGPPRSYLRQWVVIPPPLSSPGPQASEPASLSSNHSLTSCFTNQGYFFFHLPDALEIEACQVYFTYDPYSEEDPDEGVAGAPTGSSPQPLQPLSGEDDAYCTFPSRDDLLLFSEFRALNARLPLNTDAYLSLQELQGQDPTHLV

SEQ ID NO: 29 - protein, 924 aa, synthetic construct
  source 1..924: note = BM-15-21mDO (Y519) amino acid sequence
MALPVTALLLPLALLLHAARPDVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLEPASLSSNHSLTSCFTNQGYFFFHLPDALEIEACQVYFTYDPYSEEDPDEGVAGAPTGSSPQPLQPLSGEDDAYCTFPSRDDLLLFSEFRALNARLPLNTDAYLSLQELQGQDPTHLVECDFTSPGDEGPPRSTLRQWVVIPPPLSSPGPQAS

SEQ ID NO: 30 - protein, 924 aa, synthetic construct
  source 1..924: note = BM-15mDO (Y364)-21 amino acid sequence
MALPVTALLLPLALLLHAARPDVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLEPASLSSNHSLTSCFTNQGTFFFHLPDALEIEACQVYFTYDPYSEEDPDEGVAGAPTGSSPQPLQPLSGEDDAYCTFPSRDDLLLFSEFRALNARLPLNTDAYLSLQELQGQDPTHLVECDFTSPGDEGPPRSYLRQWVVIPPPLSSPGPQAS

SEQ ID NO: 31 - protein, 924 aa, synthetic construct
  source 1..924: note = BM-15mDO (Y364, 381, 384, 387)-21 amino acid sequence
MALPVTALLLPLALLLHAARPDVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLEPASLSSNHSLTSCFTNQGTFFFHLPDALEIEACQVTFTTDPTSEEDPDEGVAGAPTGSSPQPLQPLSGEDDAYCTFPSRDDLLLFSEFRALNARLPLNTDAYLSLQELQGQDPTHLVECDFTSPGDEGPPRSYLRQWVVIPPPLSSPGPQAS

SEQ ID NO: 32 - protein, 924 aa, synthetic construct
  source 1..924: note = BM-15mDO (Y418)-21 amino acid sequence
MALPVTALLLPLALLLHAARPDVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLEPASLSSNHSLTSCFTNQGYFFFHLPDALEIEACQVYFTYDPYSEEDPDEGVAGAPTGSSPQPLQPLSGEDDATCTFPSRDDLLLFSEFRALNARLPLNTDAYLSLQELQGQDPTHLVECDFTSPGDEGPPRSYLRQWVVIPPPLSSPGPQAS

SEQ ID NO: 33 - protein, 924 aa, synthetic construct
  source 1..924: note = BM-15mDO (Y536)-21 amino acid sequence
MALPVTALLLPLALLLHAARPDVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLEPASLSSNHSLTSCFTNQGYFFFHLPDALEIEACQVYFTYDPYSEEDPDEGVAGAPTGSSPQPLQPLSGEDDAYCTFPSRDDLLLFSEFRALNARLPLNTDATLSLQELQGQDPTHLVECDFTSPGDEGPPRSYLRQWVVIPPPLSSPGPQAS

SEQ ID NO: 34 - protein, 924 aa, synthetic construct
  source 1..924: note = BM-15mDO (Y418, 536)-21 amino acid sequence
MALPVTALLLPLALLLHAARPDVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLEPASLSSNHSLTSCFTNQGYFFFHLPDALEIEACQVYFTYDPYSEEDPDEGVAGAPTGSSPQPLQPLSGEDDATCTFPSRDDLLLFSEFRALNARLPLNTDATLSLQELQGQDPTHLVECDFTSPGDEGPPRSYLRQWVVIPPPLSSPGPQAS

SEQ ID NO: 35 - protein, 924 aa, synthetic construct
  source 1..924: note = BM-15mDO (Y364, 381, 384, 387, 418, 536F)-21 amino acid sequence
MALPVTALLLPLALLLHAARPDVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLEPASLSSNHSLTSCFTNQGTFFFHLPDALEIEACQVTFTTDPTSEEDPDEGVAGAPTGSSPQPLQPLSGEDDATCTFPSRDDLLLFSEFRALNARLPLNTDATLSLQELQGQDPTHLVECDFTSPGDEGPPRSYLRQWVVIPPPLSSPGPQAS

SEQ ID NO: 36 - protein, 893 aa, synthetic construct
  source 1..893: note = BM-NKG2D-TPOR TM-TPOR (BOX1-BOX2)-IL15Rbeta truncated1-IL21R amino acid sequence
MALPVTALLLPLALLLHAARPDVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLEPASLSSNHSLTSCFTNQGYFFFHLPDALEIEACQVYFTYDPYSEEDPDEGVAGAPTGSSPQPLQPLSGEDDAYCTFPSRDDLLLFSECDFTSPGDEGPPRSYLRQWVVIPPPLSSPGPQAS

SEQ ID NO: 37 - protein, 866 aa, synthetic construct
  source 1..866: note = BM-NKG2D-TPOR TM-TPOR (BOX1-BOX2)-IL15Rbeta truncated2-IL21R amino acid sequence
MALPVTALLLPLALLLHAARPDVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLEPASLSSNHSLTSCFTNQGYFFFHLPDALEIEACQVYFTYDPYSEEDPDEGVAGAPTGSSECDFTSPGDEGPPRSYLRQWVVIPPPLSSPGPQAS

SEQ ID NO: 38 - protein, 5 aa, synthetic construct
  source 1..5: note = GPC3 BM scFv VH CDR1
DYEMH

SEQ ID NO: 39 - protein, 17 aa, synthetic construct
  source 1..17: note = GPC3 BM scFv VH CDR2
ALDPKTGDTAYSQKFKG

SEQ ID NO: 40 - protein, 6 aa, synthetic construct
  source 1..6: note = GPC3 BM scFv VH CDR3
FYSYTY

SEQ ID NO: 41 - protein, 16 aa, synthetic construct
  source 1..16: note = GPC3 BM scFv VL CDR1
RSSQSLVHSNANTYLH

SEQ ID NO: 42 - protein, 7 aa, synthetic construct
  source 1..7: note = GPC3 BM scFv VL CDR2
KVSNRFS

SEQ ID NO: 43 - protein, 9 aa, synthetic construct
  source 1..9: note = GPC3 BM scFv VL CDR3
SQNTHVPPT

SEQ ID NO: 44 - protein, 5 aa, synthetic construct
  source 1..5: note = 8E52I scFv VH CDR1
GYNWH

SEQ ID NO: 45 - protein, 16 aa, synthetic construct
  source 1..16: note = 8E52I scFv VH CDR2
YIHYTGSTNYNPALRS

SEQ ID NO: 46 - protein, 9 aa, synthetic construct
  source 1..9: note = 8E52I scFv VH CDR3
IYNGNSFPY

SEQ ID NO: 47 - protein, 17 aa, synthetic construct
  source 1..17: note = 8E52I scFv VL CDR1
KSSQSLFNSGNQKNYLT

SEQ ID NO: 48 - protein, 7 aa, synthetic construct
  source 1..7: note = 8E52I scFv VL CDR2
WASTRES

SEQ ID NO: 49 - protein, 9 aa, synthetic construct
  source 1..9: note = 8E52I scFv VL CDR3
QNAYSFPYT

SEQ ID NO: 50 - protein, 5 aa, synthetic construct
  source 1..5: note = IMB-362 scFv VH CDR1
SYWIN

SEQ ID NO: 51 - protein, 17 aa, synthetic construct
  source 1..17: note = IMB-362 scFv VH CDR2
NIYPSDSYTNYNQKFKD

SEQ ID NO: 52 - protein, 9 aa, synthetic construct
  source 1..9: note = IMB-362 scFv VH CDR3
SWRGNSFDY

SEQ ID NO: 53 - protein, 17 aa, synthetic construct
  source 1..17: note = IMB-362 scFv VL CDR1
KSSQSLLNSGNQKNYLT

SEQ ID NO: 54 - protein, 7 aa, synthetic construct
  source 1..7: note = IMB-362 scFv VL CDR2
WASTRES

SEQ ID NO: 55 - protein, 9 aa, synthetic construct
  source 1..9: note = IMB-362 scFv VL CDR3
QNDYSYPFT

SEQ ID NO: 56 - protein, 5 aa, synthetic construct
  source 1..5: note = LIC182508 sdAb CDR1
SYCMS

SEQ ID NO: 57 - protein, 18 aa, synthetic construct
  source 1..18: note = LIC182508 sdAb CDR2
AISTRGGSTYYVDSVKGR

SEQ ID NO: 58 - protein, 18 aa, synthetic construct
  source 1..18: note = LIC182508 sdAb CDR3
AEGLCGWGGWESAVRFGS

SEQ ID NO: 59 - protein, 5 aa, synthetic construct
  source 1..5: note = LIC182511 sdAb CDR1
TYCMG

SEQ ID NO: 60 - protein, 19 aa, synthetic construct
  source 1..19: note = LIC182511 sdAb CDR2
ANVYMRNGDTWLADSVKGR

SEQ ID NO: 61 - protein, 12 aa, synthetic construct
  source 1..12: note = LIC182511 sdAb CDR3
GRSSCLEASISY

SEQ ID NO: 62 - protein, 5 aa, synthetic construct
  source 1..5: note = C18S LIC182513 sdAb CDR1
SVCMG

SEQ ID NO: 63 - protein, 18 aa, synthetic construct
  source 1..18: note = C18S LIC182513 sdAb CDR2
AVIGRDGSTTYIDSVKGR

SEQ ID NO: 64 - protein, 11 aa, synthetic construct
  source 1..11: note = C18S LIC182513 sdAb CDR3
GLGYWACEYNY

SEQ ID NO: 65 - protein, 5 aa, synthetic construct
  source 1..5: note = LIC182514 sdAb CDR1
SKCMA

SEQ ID NO: 66 - protein, 19 aa, synthetic construct
  source 1..19: note = LIC182514 sdAb CDR2
ATIQSGYYRPYYTDSVKGR

SEQ ID NO: 67 - protein, 20 aa, synthetic construct
  source 1..20: note = LIC182514 sdAb CDR3
DRFTYPYGVICGGISANYRY

SEQ ID NO: 68 - protein, 127 aa, synthetic construct
  source 1..127: note = LIC182508 sdAb
EVQLAESGGGSVQAGGSLRLSCAASRYISSSYCMSWFRQAPGKEREGVAAISTRGGSTYYVDSVKGRFTIFQGNAGATVYLQMNSLKPEDTAMYYCAAAEGLCGWGGWESAVRFGSWGQGTQVTVSS

SEQ ID NO: 69 - protein, 118 aa, synthetic construct
  source 1..118: note = LIC182511 sdAb
QVQLDESGGGSVQAGGSLRLSCAASRSTYCMGWFRQAPGKLRERVANVYMRNGDTWLADSVKGRFTISRDNAKNAVYLQMTRLRPEDTAMYYCAAGRSSCLEASISYWGQGTQVTVSS

SEQ ID NO: 70 - protein, 118 aa, synthetic construct
  source 1..118: note = C18S LIC182513 sdAb
QVQLEESGGGSVQVGGSLRLSCAASGYRSSVCMGWFRQAPGKERERVAVIGRDGSTTYIDSVKGRFTISRDSAKNTLSLQMDNLKPEDTAMYSCAAGLGYWACEYNYWGQGTQVTVSS

SEQ ID NO: 71 - protein, 129 aa, synthetic construct
  source 1..129: note = LIC182514 sdAb
QVQLVESGGGSVQAGGSLSLSCAASGDTFHSKCMAWFRQGPGKEREVVATIQSGYYRPYYTDSVKGRFTISRDADKNRVYLQMDSLKPEDTAMYYCAADRFTYPYGVICGGISANYRYWGQGTRVTVSS

SEQ ID NO: 72 - protein, 115 aa, synthetic construct
  source 1..115: note = GPC3 BM scFv VH
QVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSS

SEQ ID NO: 73 - protein, 113 aa, synthetic construct
  source 1..113: note = GPC3 BM scFv VL
DVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKR

SEQ ID NO: 74 - protein, 118 aa, synthetic construct
  source 1..118: note = 8E52I scFv VH
QVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSS

SEQ ID NO: 75 - protein, 114 aa, synthetic construct
  source 1..114: note = 8E52I scFv VL
DIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKR

SEQ ID NO: 76 - protein, 118 aa, synthetic construct
  source 1..118: note = IMB-362 scFv VH
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSS

SEQ ID NO: 77 - protein, 114 aa, synthetic construct
  source 1..114: note = IMB-362 scFv VL
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKR

SEQ ID NO: 78 - protein, 350 aa, synthetic construct
  source 1..350: note = LIC182508 CAR
EVQLAESGGGSVQAGGSLRLSCAASRYISSSYCMSWFRQAPGKEREGVAAISTRGGSTYYVDSVKGRFTIFQGNAGATVYLQMNSLKPEDTAMYYCAAAEGLCGWGGWESAVRFGSWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 79 - protein, 341 aa, synthetic construct
  source 1..341: note = LIC182511 CAR
QVQLDESGGGSVQAGGSLRLSCAASRSTYCMGWFRQAPGKLRERVANVYMRNGDTWLADSVKGRFTISRDNAKNAVYLQMTRLRPEDTAMYYCAAGRSSCLEASISYWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 80 - protein, 341 aa, synthetic construct
  source 1..341: note = C18S LIC182513 CAR
QVQLEESGGGSVQVGGSLRLSCAASGYRSSVCMGWFRQAPGKERERVAVIGRDGSTTYIDSVKGRFTISRDSAKNTLSLQMDNLKPEDTAMYSCAAGLGYWACEYNYWGQGTQVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 81 - protein, 352 aa, synthetic construct
  source 1..352: note = LIC182514 CAR
QVQLVESGGGSVQAGGSLSLSCAASGDTFHSKCMAWFRQGPGKEREVVATIQSGYYRPYYTDSVKGRFTISRDADKNRVYLQMDSLKPEDTAMYYCAADRFTYPYGVICGGISANYRYWGQGTRVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 82 - protein, 470 aa, synthetic construct
  source 1..470: note = 8E52I CAR
DIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKRGGGGSGGGGSGGGGSQVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 83 - protein, 470 aa, synthetic construct
  source 1..470: note = IMB-362 CAR
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKRGGGGSGGGGSGGGGSQVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 84 - protein, 5 aa, synthetic construct
  source 1..5: note = Linker peptide
GSGGS

SEQ ID NO: 85 - protein, 4 aa, synthetic construct
  source 1..4: note = Linker peptide
GGGS

SEQ ID NO: 86 - protein, 45 aa, synthetic construct
  source 1..45: note = CD8 Hinge region amino acid sequence
TTTPAPRPPTPAPTIASQPLSLRPEACRPAAGGAVHTRGLDFACD

SEQ ID NO: 87 - protein, 286 aa, synthetic construct
  source 1..286: note = IL15Rbeta full intracellular domain amino acid sequence
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 88 - protein, 285 aa, synthetic construct
  source 1..285: note = IL21R full intracellular domain amino acid sequence
SLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 89 - protein, 538 aa, synthetic construct
  source 1..538: note = IL21R full-length amino acid sequence
MPRGWAAPLLLLLLQGGWGCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 90 - protein, 551 aa, synthetic construct
  source 1..551: note = IL15Rbeta full-length amino acid sequence
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 91 - protein, 464 aa, synthetic construct
  source 1..464: note = AZ-CAR 4-1BB CAR amino acid sequence
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGKRYFDYWGQGTMVTVSSGGGGSGGGGSGGGGSQSVLTQPPSASGTPGQRVTISCSGGSSDIGSNTVNWYQQLPGTAPKLLIYYNNQRPSGVPDRFSGSKSGTSASLAISGLQSEDEADYYCATWDDRMYSPVFGGGTKLTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 92 - protein, 922 aa, synthetic construct
  source 1..922: note = AZ-CAR-B6 amino acid sequence
MALPVTALLLPLALLLHAARPEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGKRYFDYWGQGTMVTVSSGGGGSGGGGSGGGGSQSVLTQPPSASGTPGQRVTISCSGGSSDIGSNTVNWYQQLPGTAPKLLIYYNNQRPSGVPDRFSGSKSGTSASLAISGLQSEDEADYYCATWDDRMYSPVFGGGTKLTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLEPASLSSNHSLTSCFTNQGYFFFHLPDALEIEACQVYFTYDPYSEEDPDEGVAGAPTGSSPQPLQPLSGEDDAYCTFPSRDDLLLFSEFRALNARLPLNTDAYLSLQELQGQDPTHLVECDFTSPGDEGPPRSYLRQWVVIPPPLSSPGPQAS

SEQ ID NO: 93 - protein, 908 aa, synthetic construct
  source 1..908: note = AZ-CAR-B6M2 amino acid sequence
MALPVTALLLPLALLLHAARPEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGKRYFDYWGQGTMVTVSSGGGGSGGGGSGGGGSQSVLTQPPSASGTPGQRVTISCSGGSSDIGSNTVNWYQQLPGTAPKLLIYYNNQRPSGVPDRFSGSKSGTSASLAISGLQSEDEADYYCATWDDRMYSPVFGGGTKLTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVHLCPSPLFPGPSKPFWVLVVVGGVLACYSLLVTVAFIIFWVRWQFPAHYRRLRHALWPSLPDLHRVLGQYLRDTAALSPPKATVSDTCEEVEPASLSSNHSLTSCFTNQGYFFFHLPDALEIEACQVYFTYDPYSEEDPDEGVAGAPTGSSPQPLQPLSGEDDAYCTFPSRDDLLLFSEFRALNARLPLNTDAYLSLQELQGQDPTHLVECDFTSPGDEGPPRSYLRQWVVIPPPLSSPGPQAS

SEQ ID NO: 94 - protein, 116 aa, synthetic construct
  source 1..116: note = GPC3 AZ-CAR scFv VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGKRYFDYWGQGTMVTVSS

SEQ ID NO: 95 - protein, 110 aa, synthetic construct
  source 1..110: note = GPC3 AZ-CAR scFv VL
QSVLTQPPSASGTPGQRVTISCSGGSSDIGSNTVNWYQQLPGTAPKLLIYYNNQRPSGVPDRFSGSKSGTSASLAISGLQSEDEADYYCATWDDRMYSPVFGGGTKLTVL

SEQ ID NO: 96 - protein, 25 aa, synthetic construct
  source 1..25: note = DAP10 TM 49-73 amino acid sequence
LLAGLVAADAVASLLIVGAVFLCAR

SEQ ID NO: 97 - protein, 5 aa, synthetic construct
  source 1..5: note = GPC3 AZ-CAR scFv VH CDR1
SYAMS

SEQ ID NO: 98 - protein, 17 aa, synthetic construct
  source 1..17: note = GPC3 AZ-CAR scFv VH CDR2
AISGSGGSTYYADSVKG

SEQ ID NO: 99 - protein, 7 aa, synthetic construct
  source 1..7: note = GPC3 AZ-CAR scFv VH CDR3
GKRYFDY

SEQ ID NO: 100 - protein, 13 aa, synthetic construct
  source 1..13: note = GPC3 AZ-CAR scFv VL CDR1
SGGSSDIGSNTVN

SEQ ID NO: 101 - protein, 6 aa, synthetic construct
  source 1..6: note = GPC3 AZ-CAR scFv VL CDR2
NNQRPS

SEQ ID NO: 102 - protein, 11 aa, synthetic construct
  source 1..11: note = GPC3 AZ-CAR scFv VL CDR3
ATWDDRMYSPV

SEQ ID NO: 103 - protein, 394 aa, synthetic construct
  source 1..394: note = B6=NKG2D-TPOR TM-TPOR (BOX1-BOX2)-IL15Rbeta-IL21R without signal peptide
SLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLEPASLSSNHSLTSCFTNQGYFFFHLPDALEIEACQVYFTYDPYSEEDPDEGVAGAPTGSSPQPLQPLSGEDDAYCTFPSRDDLLLFSEFRALNARLPLNTDAYLSLQELQGQDPTHLVECDFTSPGDEGPPRSYLRQWVVIPPPLSSPGPQAS

SEQ ID NO: 104 - protein, 415 aa, synthetic construct
  source 1..415: note = B6=NKG2D-TPOR TM-TPOR (BOX1-BOX2)-IL15Rbeta-IL21R with signal peptide
MALPVTALLLPLALLLHAARPSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLEPASLSSNHSLTSCFTNQGYFFFHLPDALEIEACQVYFTYDPYSEEDPDEGVAGAPTGSSPQPLQPLSGEDDAYCTFPSRDDLLLFSEFRALNARLPLNTDAYLSLQELQGQDPTHLVECDFTSPGDEGPPRSYLRQWVVIPPPLSSPGPQAS

SEQ ID NO: 105 - protein, 336 aa, synthetic construct
  source 1..336: note = B6M1=NKG2D-TPOR TM-TPOR (BOX1-BOX2)-IL-15Rbeta Truncated2-IL-21R without signal peptide
SLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLEPASLSSNHSLTSCFTNQGYFFFHLPDALEIEACQVYFTYDPYSEEDPDEGVAGAPTGSSECDFTSPGDEGPPRSYLRQWVVIPPPLSSPGPQAS

SEQ ID NO: 106 - protein, 357 aa, synthetic construct
  source 1..357: note = B6M1= NKG2D-TPOR TM-TPOR (BOX1-BOX2)-IL-15Rbeta Truncated2-IL-21R with signal peptide
MALPVTALLLPLALLLHAARPSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLEPASLSSNHSLTSCFTNQGYFFFHLPDALEIEACQVYFTYDPYSEEDPDEGVAGAPTGSSECDFTSPGDEGPPRSYLRQWVVIPPPLSSPGPQAS

SEQ ID NO: 107 - protein, 380 aa, synthetic construct
  source 1..380: note = B6M2=NKG2D-CD28 TM-TPOR (BOX1)-IL-15Rbeta-IL-21R without signal peptide
SLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVHLCPSPLFPGPSKPFWVLVVVGGVLACYSLLVTVAFIIFWVRWQFPAHYRRLRHALWPSLPDLHRVLGQYLRDTAALSPPKATVSDTCEEVEPASLSSNHSLTSCFTNQGYFFFHLPDALEIEACQVYFTYDPYSEEDPDEGVAGAPTGSSPQPLQPLSGEDDAYCTFPSRDDLLLFSEFRALNARLPLNTDAYLSLQELQGQDPTHLVECDFTSPGDEGPPRSYLRQWVVIPPPLSSPGPQAS

SEQ ID NO: 108 - protein, 401 aa, synthetic construct
  source 1..401: note = B6M2=NKG2D-CD28 TM-TPOR (BOX1)-IL-15Rbeta-IL-21R with signal peptide
MALPVTALLLPLALLLHAARPSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVHLCPSPLFPGPSKPFWVLVVVGGVLACYSLLVTVAFIIFWVRWQFPAHYRRLRHALWPSLPDLHRVLGQYLRDTAALSPPKATVSDTCEEVEPASLSSNHSLTSCFTNQGYFFFHLPDALEIEACQVYFTYDPYSEEDPDEGVAGAPTGSSPQPLQPLSGEDDAYCTFPSRDDLLLFSEFRALNARLPLNTDAYLSLQELQGQDPTHLVECDFTSPGDEGPPRSYLRQWVVIPPPLSSPGPQAS

SEQ ID NO: 109 - protein, 363 aa, synthetic construct
  source 1..363: note = B6M3=NKG2D-TPOR TM-TPOR (BOX1-BOX2)-IL-15Rbeta Truncated1-IL-21R without signal peptide
SLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLEPASLSSNHSLTSCFTNQGYFFFHLPDALEIEACQVYFTYDPYSEEDPDEGVAGAPTGSSPQPLQPLSGEDDAYCTFPSRDDLLLFSECDFTSPGDEGPPRSYLRQWVVIPPPLSSPGPQAS

SEQ ID NO: 110 - protein, 384 aa, synthetic construct
  source 1..384: note = B6M3=NKG2D-TPOR TM-TPOR (BOX1-BOX2)-IL-15Rbeta Truncated1-IL-21R with signal peptide
MALPVTALLLPLALLLHAARPSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLEPASLSSNHSLTSCFTNQGYFFFHLPDALEIEACQVYFTYDPYSEEDPDEGVAGAPTGSSPQPLQPLSGEDDAYCTFPSRDDLLLFSECDFTSPGDEGPPRSYLRQWVVIPPPLSSPGPQAS

SEQ ID NO: 111 - protein, 399 aa, synthetic construct
  source 1..399: note = B6M4=NKG2D-CD28 TM-TPOR (BOX1-BOX2)-IL-15Rbeta-IL-21R without signal peptide
SLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVHLCPSPLFPGPSKPFWVLVVVGGVLACYSLLVTVAFIIFWVRWQFPAHYRRLRHALWPSLPDLHRVLGQYLRDTAALSPPKATVSDTCEEVEPSLLEILPKSSERTPLPLEPASLSSNHSLTSCFTNQGYFFFHLPDALEIEACQVYFTYDPYSEEDPDEGVAGAPTGSSPQPLQPLSGEDDAYCTFPSRDDLLLFSEFRALNARLPLNTDAYLSLQELQGQDPTHLVECDFTSPGDEGPPRSYLRQWVVIPPPLSSPGPQAS

SEQ ID NO: 112 - protein, 420 aa, synthetic construct
  source 1..420: note = B6M4=NKG2D-CD28 TM-TPOR (BOX1-BOX2)-IL-15Rbeta-IL-21R with signal peptide
MALPVTALLLPLALLLHAARPSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVHLCPSPLFPGPSKPFWVLVVVGGVLACYSLLVTVAFIIFWVRWQFPAHYRRLRHALWPSLPDLHRVLGQYLRDTAALSPPKATVSDTCEEVEPSLLEILPKSSERTPLPLEPASLSSNHSLTSCFTNQGYFFFHLPDALEIEACQVYFTYDPYSEEDPDEGVAGAPTGSSPQPLQPLSGEDDAYCTFPSRDDLLLFSEFRALNARLPLNTDAYLSLQELQGQDPTHLVECDFTSPGDEGPPRSYLRQWVVIPPPLSSPGPQAS

SEQ ID NO: 113 - protein, 427 aa, synthetic construct
  source 1..427: note = NKG2D-CD8 TM-TPOR (BOX1-BOX2)-IL15Rbeta-IL21R without signal peptide
SLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCRWQFPAHYRRLRHALWPSLPDLHRVLGQYLRDTAALSPPKATVSDTCEEVEPSLLEILPKSSERTPLPLEPASLSSNHSLTSCFTNQGYFFFHLPDALEIEACQVYFTYDPYSEEDPDEGVAGAPTGSSPQPLQPLSGEDDAYCTFPSRDDLLLFSEFRALNARLPLNTDAYLSLQELQGQDPTHLVECDFTSPGDEGPPRSYLRQWVVIPPPLSSPGPQAS

SEQ ID NO: 114 - protein, 448 aa, synthetic construct
  source 1..448: note = NKG2D-CD8 TM-TPOR (BOX1-BOX2)-IL15Rbeta-IL21R with signal peptide
MALPVTALLLPLALLLHAARPSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCRWQFPAHYRRLRHALWPSLPDLHRVLGQYLRDTAALSPPKATVSDTCEEVEPSLLEILPKSSERTPLPLEPASLSSNHSLTSCFTNQGYFFFHLPDALEIEACQVYFTYDPYSEEDPDEGVAGAPTGSSPQPLQPLSGEDDAYCTFPSRDDLLLFSEFRALNARLPLNTDAYLSLQELQGQDPTHLVECDFTSPGDEGPPRSYLRQWVVIPPPLSSPGPQAS

SEQ ID NO: 115 - protein, 375 aa, synthetic construct
  source 1..375: note = NKG2D-TPOR TM-TPOR (BOX1)-IL15Rbeta-IL21R without signal peptide
SLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLRWQFPAHYRRLRHALWPSLPDLHRVLGQYLRDTAALSPPKATVSDTCEEVEPASLSSNHSLTSCFTNQGYFFFHLPDALEIEACQVYFTYDPYSEEDPDEGVAGAPTGSSPQPLQPLSGEDDAYCTFPSRDDLLLFSEFRALNARLPLNTDAYLSLQELQGQDPTHLVECDFTSPGDEGPPRSYLRQWVVIPPPLSSPGPQAS

SEQ ID NO: 116 - protein, 396 aa, synthetic construct
  source 1..396: note = NKG2D-TPOR TM-TPOR (BOX1)-IL15Rbeta-IL21R with signal peptide
MALPVTALLLPLALLLHAARPSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLRWQFPAHYRRLRHALWPSLPDLHRVLGQYLRDTAALSPPKATVSDTCEEVEPASLSSNHSLTSCFTNQGYFFFHLPDALEIEACQVYFTYDPYSEEDPDEGVAGAPTGSSPQPLQPLSGEDDAYCTFPSRDDLLLFSEFRALNARLPLNTDAYLSLQELQGQDPTHLVECDFTSPGDEGPPRSYLRQWVVIPPPLSSPGPQAS

SEQ ID NO: 117 - protein, 397 aa, synthetic construct
  source 1..397: note = NKG2D-TPOR TM-IL15Rbeta (BOX1-BOX2)-IL15Rbeta-IL21R without signal peptide
SLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLNCRNTGPWLKKVLKCNTPDPSKFFSQLSSEHGGDVQKWLSSPFPSSSFSPGGLAPEISPLEVLERDKVTQLLEPASLSSNHSLTSCFTNQGYFFFHLPDALEIEACQVYFTYDPYSEEDPDEGVAGAPTGSSPQPLQPLSGEDDAYCTFPSRDDLLLFSEFRALNARLPLNTDAYLSLQELQGQDPTHLVECDFTSPGDEGPPRSYLRQWVVIPPPLSSPGPQAS

SEQ ID NO: 118 - protein, 418 aa, synthetic construct
  source 1..418: note = NKG2D-TPOR TM-IL15Rbeta (BOX1-BOX2)-IL15Rbeta-IL21R with signal peptide
MALPVTALLLPLALLLHAARPSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLNCRNTGPWLKKVLKCNTPDPSKFFSQLSSEHGGDVQKWLSSPFPSSSFSPGGLAPEISPLEVLERDKVTQLLEPASLSSNHSLTSCFTNQGYFFFHLPDALEIEACQVYFTYDPYSEEDPDEGVAGAPTGSSPQPLQPLSGEDDAYCTFPSRDDLLLFSEFRALNARLPLNTDAYLSLQELQGQDPTHLVECDFTSPGDEGPPRSYLRQWVVIPPPLSSPGPQAS

SEQ ID NO: 119 - protein, 394 aa, synthetic construct
  source 1..394: note = NKG2D-TPOR TM-TPOR (BOX1-BOX2)-IL21R-IL15Rbeta without signal peptide
SLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLECDFTSPGDEGPPRSYLRQWVVIPPPLSSPGPQASEPASLSSNHSLTSCFTNQGYFFFHLPDALEIEACQVYFTYDPYSEEDPDEGVAGAPTGSSPQPLQPLSGEDDAYCTFPSRDDLLLFSEFRALNARLPLNTDAYLSLQELQGQDPTHLV

SEQ ID NO: 120 - protein, 415 aa, synthetic construct
  source 1..415: note = NKG2D-TPOR TM-TPOR (BOX1-BOX2)-IL21R-IL15Rbeta with signal peptide
MALPVTALLLPLALLLHAARPSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLECDFTSPGDEGPPRSYLRQWVVIPPPLSSPGPQASEPASLSSNHSLTSCFTNQGYFFFHLPDALEIEACQVYFTYDPYSEEDPDEGVAGAPTGSSPQPLQPLSGEDDAYCTFPSRDDLLLFSEFRALNARLPLNTDAYLSLQELQGQDPTHLV

SEQ ID NO: 121 - protein, 394 aa, synthetic construct
  source 1..394: note = 15-21mDO (Y519) without signal peptide
SLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLEPASLSSNHSLTSCFTNQGYFFFHLPDALEIEACQVYFTYDPYSEEDPDEGVAGAPTGSSPQPLQPLSGEDDAYCTFPSRDDLLLFSEFRALNARLPLNTDAYLSLQELQGQDPTHLVECDFTSPGDEGPPRSTLRQWVVIPPPLSSPGPQAS

SEQ ID NO: 122 - protein, 415 aa, synthetic construct
  source 1..415: note = 15-21mDO (Y519) with signal peptide
MALPVTALLLPLALLLHAARPSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLEPASLSSNHSLTSCFTNQGYFFFHLPDALEIEACQVYFTYDPYSEEDPDEGVAGAPTGSSPQPLQPLSGEDDAYCTFPSRDDLLLFSEFRALNARLPLNTDAYLSLQELQGQDPTHLVECDFTSPGDEGPPRSTLRQWVVIPPPLSSPGPQAS

SEQ ID NO: 123 - protein, 394 aa, synthetic construct
  source 1..394: note = 15mDO (Y364)-21 without signal peptide
SLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLEPASLSSNHSLTSCFTNQGTFFFHLPDALEIEACQVYFTYDPYSEEDPDEGVAGAPTGSSPQPLQPLSGEDDAYCTFPSRDDLLLFSEFRALNARLPLNTDAYLSLQELQGQDPTHLVECDFTSPGDEGPPRSYLRQWVVIPPPLSSPGPQAS

SEQ ID NO: 124 - protein, 415 aa, synthetic construct
  source 1..415: note = 15mDO (Y364)-21 with signal peptide
MALPVTALLLPLALLLHAARPSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLEPASLSSNHSLTSCFTNQGTFFFHLPDALEIEACQVYFTYDPYSEEDPDEGVAGAPTGSSPQPLQPLSGEDDAYCTFPSRDDLLLFSEFRALNARLPLNTDAYLSLQELQGQDPTHLVECDFTSPGDEGPPRSYLRQWVVIPPPLSSPGPQAS

SEQ ID NO: 125 - protein, 394 aa, synthetic construct
  source 1..394: note = 15mDO (Y364, 381, 384, 387)-21 without signal peptide
SLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLEPASLSSNHSLTSCFTNQGTFFFHLPDALEIEACQVTFTTDPTSEEDPDEGVAGAPTGSSPQPLQPLSGEDDAYCTFPSRDDLLLFSEFRALNARLPLNTDAYLSLQELQGQDPTHLVECDFTSPGDEGPPRSYLRQWVVIPPPLSSPGPQAS

SEQ ID NO: 126 - protein, 415 aa, synthetic construct
  source 1..415: note = 15mDO (Y364, 381, 384, 387)-21 with signal peptide
MALPVTALLLPLALLLHAARPSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLEPASLSSNHSLTSCFTNQGTFFFHLPDALEIEACQVTFTTDPTSEEDPDEGVAGAPTGSSPQPLQPLSGEDDAYCTFPSRDDLLLFSEFRALNARLPLNTDAYLSLQELQGQDPTHLVECDFTSPGDEGPPRSYLRQWVVIPPPLSSPGPQAS

SEQ ID NO: 127 - protein, 394 aa, synthetic construct
  source 1..394: note = 15mDO (Y418)-21 without signal peptide
SLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLEPASLSSNHSLTSCFTNQGYFFFHLPDALEIEACQVYFTYDPYSEEDPDEGVAGAPTGSSPQPLQPLSGEDDATCTFPSRDDLLLFSEFRALNARLPLNTDAYLSLQELQGQDPTHLVECDFTSPGDEGPPRSYLRQWVVIPPPLSSPGPQAS

SEQ ID NO: 128 - protein, 415 aa, synthetic construct
  source 1..415: note = 15mDO (Y418)-21 with signal peptide
MALPVTALLLPLALLLHAARPSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLEPASLSSNHSLTSCFTNQGYFFFHLPDALEIEACQVYFTYDPYSEEDPDEGVAGAPTGSSPQPLQPLSGEDDATCTFPSRDDLLLFSEFRALNARLPLNTDAYLSLQELQGQDPTHLVECDFTSPGDEGPPRSYLRQWVVIPPPLSSPGPQAS

SEQ ID NO: 129 - protein, 394 aa, synthetic construct
  source 1..394: note = 15mDO (Y536)-21 without signal peptide
SLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLEPASLSSNHSLTSCFTNQGYFFFHLPDALEIEACQVYFTYDPYSEEDPDEGVAGAPTGSSPQPLQPLSGEDDAYCTFPSRDDLLLFSEFRALNARLPLNTDATLSLQELQGQDPTHLVECDFTSPGDEGPPRSYLRQWVVIPPPLSSPGPQAS

SEQ ID NO: 130 - protein, 415 aa, synthetic construct
  source 1..415: note = 15mDO (Y536)-21 with signal peptide
MALPVTALLLPLALLLHAARPSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLEPASLSSNHSLTSCFTNQGYFFFHLPDALEIEACQVYFTYDPYSEEDPDEGVAGAPTGSSPQPLQPLSGEDDAYCTFPSRDDLLLFSEFRALNARLPLNTDATLSLQELQGQDPTHLVECDFTSPGDEGPPRSYLRQWVVIPPPLSSPGPQAS

SEQ ID NO: 131 - protein, 394 aa, synthetic construct
  source 1..394: note = 15mDO (Y418, 536)-21 without signal peptide
SLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLEPASLSSNHSLTSCFTNQGYFFFHLPDALEIEACQVYFTYDPYSEEDPDEGVAGAPTGSSPQPLQPLSGEDDATCTFPSRDDLLLFSEFRALNARLPLNTDATLSLQELQGQDPTHLVECDFTSPGDEGPPRSYLRQWVVIPPPLSSPGPQAS

SEQ ID NO: 132 - protein, 415 aa, synthetic construct
  source 1..415: note = 15mDO (Y418, 536)-21 with signal peptide
MALPVTALLLPLALLLHAARPSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLEPASLSSNHSLTSCFTNQGYFFFHLPDALEIEACQVYFTYDPYSEEDPDEGVAGAPTGSSPQPLQPLSGEDDATCTFPSRDDLLLFSEFRALNARLPLNTDATLSLQELQGQDPTHLVECDFTSPGDEGPPRSYLRQWVVIPPPLSSPGPQAS

SEQ ID NO: 133 - protein, 394 aa, synthetic construct
  source 1..394: note = 15mDO (Y364, 381, 384, 387, 418, 536F)-21 without signal peptide
SLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLEPASLSSNHSLTSCFTNQGTFFFHLPDALEIEACQVTFTTDPTSEEDPDEGVAGAPTGSSPQPLQPLSGEDDATCTFPSRDDLLLFSEFRALNARLPLNTDATLSLQELQGQDPTHLVECDFTSPGDEGPPRSYLRQWVVIPPPLSSPGPQAS

SEQ ID NO: 134 - protein, 415 aa, synthetic construct
  source 1..415: note = 15mDO (Y364, 381, 384, 387, 418, 536F)-21 with signal peptide
MALPVTALLLPLALLLHAARPSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLEPASLSSNHSLTSCFTNQGTFFFHLPDALEIEACQVTFTTDPTSEEDPDEGVAGAPTGSSPQPLQPLSGEDDATCTFPSRDDLLLFSEFRALNARLPLNTDATLSLQELQGQDPTHLVECDFTSPGDEGPPRSYLRQWVVIPPPLSSPGPQAS